(19) United States
(12) Patent Application Publication
(10) Pub. No.: US 2023/0365942 A1
(43) Pub. Date: Nov. 16, 2023

(54) ENGINEERED AAV VECTORS

(71) Applicant: LUDWIG-MAXIMILIANS-UNIVERSITÄT MÜNCHEN, München (DE)

(72) Inventors: Stylianos MICHALAKIS, München (DE); Lena ZOBEL, München (DE); Sabrina BABUTZKA, München (DE); Martin BIEL, München (DE); Hermann AMMER, München (DE)

(21) Appl. No.: 18/044,628

(22) PCT Filed: Sep. 10, 2021
(86) PCT No.: PCT/EP2021/074987

§ 371 Date: Mar. 9, 2023

(30) Foreign Application Priority Data
Sep. 10, 2020 (EP) 20195554.9

(51) Int. Cl. C12N 7/00; C07K 14/005; A61K 39/215; A61K 48/00; A61K 39/00

(52) U.S. Cl. CPC C12N 7/00; C07K 14/005; A61K 39/215; A61K 48/0008; A61K 39/0011; C12N 2770/20022; C12N 2750/14151; C12N 2750/14122; C12N 2750/14171; C12N 2750/14123; A61K 2039/5258; A61K 2039/5256; A61K 2039/585

(57) ABSTRACT

The present invention relates to an adeno-associated vims (AAV) or an adeno-associated virus-like particle (AAVLP), comprising an insert of about 75-400 amino acids in the viral proteins (VPs) VP1, VP2 and/or VP3 at an insertion site (I) at the top of variable region VIII and/or variable region IV (VR-VIII and/or VR-IV) of the VP, wherein the insert is an immunogenic protein or a portion thereof and/or wherein the insert is a protein comprising a binding domain, such as an antigen-binding domain specific for a target antigen. The present invention also relates to pharmaceutical compositions comprising said AAV or AAVLP and to the pharmaceutical composition or the AAV or AAVLP for use in therapy, particularly for use as a vaccine, for use in the treatment or the prevention of a diseases and/or for use in gene therapy. Also concerned is a method for producing the AAV of AAVLP of the present invention.

### FIELD OF THE INVENTION

The present invention relates to an adeno-associated virus (AAV) or an adeno-associated virus-like particle (AAVLP), comprising an insert of about 75-400 amino acids in the viral proteins (VPs) VP1, VP2 and/or VP3 at an insertion site (I) at the top of variable region VIII and/or variable region IV (VR-VIII and/or VR-IV) of the VP, wherein the insert is an immunogenic protein or a portion thereof and/or wherein the insert is a protein comprising a binding domain, such as an antigen-binding domain specific for a target antigen. The present invention also relates to pharmaceutical compositions comprising said AAV or AAVLP and to the pharmaceutical composition or the AAV or AAVLP for use in therapy, particularly for use as a vaccine, for use in the treatment or the prevention of a diseases and/or for use in gene therapy. Also concerned is a method for producing the AAV of AAVLP of the present invention.

### BACKGROUND OF THE INVENTION

Recombinant adeno-associated virus (AAV) vectors have proven to be a very suitable delivery system for efficient and long-term transfer of genes into human (Li, C., Samulski, R. J., Nat Rev Genet, 2020, 21: 255-272). AAVs are non-pathogenic viruses that belong to the Parvovirus family and Dependovirus genus and replicate only in the presence of adeno-, papilloma- or herpes-viruses. AAVs possess an approx. 5 kb single-stranded DNA genome (Trapani et al., Progress in retinal and eye research, 2014, Volume 43: 108-128).

AAVs have been tested extensively for safety and long-term expression of transgenes in large animal models, non-human primates and in a number of clinical trials in humans.

Structurally, AAVs are small (25 nm), non-enveloped viruses with an icosahedral capsid. The AAV capsid typically consists of 60 individual structural viral proteins (VP), in particular 5 VP1, 5 VP2 and 50 VP3 proteins. The capsid contains an approx. 4.7 kb single-stranded DNA genome comprising the two genes, Rep and Cap, between two inverted terminal repeats (ITR). Rep encodes multiple non-structural Rep proteins, which are essential for viral genome replication and packaging. Cap contains an open reading frame (ORF) that produces the structural proteins VP1, VP2 and VP3 proteins by alternative splicing and use of distinct start codons and in a ratio of approx. 1:1:10. All three VPs (VP1, VP2 and VP3) share a common sequence and only VP1 contains a unique sequence at its N terminus (approximately 138 amino acids). The position within the VP protein is therefore typically provided relative to VP1. In addition, a second +1-frameshifted ORF produces the non-structural assembly-activating protein (AAP), which acts as a chaperone that facilitates the assembly of the three VPs into the icosahedral capsid structure, without participating in the capsid structure itself. Multiple naturally occurring AAV sequence variants exist with distinct anti-AAV antibody profiles (therefore termed serotypes) (Gao et al., Curr Gene Ther., 2005, 5(3): 285-297). These multiple serotypes differ in the composition and structure of their capsid proteins and possess a varying efficiency to transduce different cell types (i.e. tropism) (Srivastava, A., Curr Opin Virol., 2016, 21: 75-80). Recombinant AAVs (rAAV) can be manufactured and purified at a high titer, making them readily available for clinical use, such as gene therapy or vaccination.

Recombinant AAVs can be produced after transfection of cell lines, such as HEK293 cells or HEK293 derived cells (e.g., HEK293T cells) with DNA plasmids encoding the Cap and Rep sequences, the AAV inverted terminal repeat (ITR)-flanked genome, and adenoviral helper sequences needed for AAV replication (Grimm et al., Human Gene Therapy, 1998, 9:18: 2745-2760). Empty AAV particles (AAVLP) can be produced in the same way when the AAV ITR-flanked genome plasmid is excluded during production (Gao et al., Mol Ther Methods Clin Dev. 2014; 1(9)).

AAV capsids tolerate insertion of peptides (up to a size of about 34 amino acids) in specific surface-exposed positions without losing their structural integrity and principle function (see e.g., WO 2012/031760 A1, WO 2998/1145401 A2 and EP 3 527 223, WO 2016/054554 A1). The VPs have nine so-called variable regions (VRs) of which VR-IV, -V and VIII form loops at the top of a protrusion. Known insertion sites in AAV2 are I-587 (e.g. insertion between the amino acid residue asparagine (N) 587 and arginine (R) 588 of AAV2 VP1) and I-453 (e.g. insertion between the amino acid residue glycine (G) 453 and threonine (T) 454 of AAV2 VP1). Such engineering of the AAV capsid had been explored for changing the AAV tropism and for re-directing the AAVs to specific cell types other than those normally infected by naturally occurring AAV serotypes (Buning, H and Srivastava, A., Mol Ther Methods Clin Dev., 2019, 12: 248-265). Thus, small peptides inserted into surface-exposed positions of AAV capsids displayed at the surface of the AAV particle have been used to change the AAV tropism. Alternatively, larger peptides have been introduced at the N-terminus of the capsid protein, but this may result in loss of VP3 expression and decreased infectivity (Warrington et al., Journal of Virology (2004), 78(12): 6595-6609). Thus, there remains a need for new strategies to introduce larger inserts and possibly re-direct the AAVs to potential target cell types.

In December 2019 a new and highly pathogenic coronavirus caused an outbreak in Wuhan city, China and quickly spread to other countries around the world. Coronaviruses are positive-sense single-stranded RNA viruses belonging to the family Coronaviridae. These viruses mostly infect animals, including birds and mammals. In humans, coronaviruses typically cause mild respiratory infections. Since 2003 already two highly pathogenic human Coronaviruses, Severe Acute Respiratory Syndrome Coronavirus (SARS-CoV) and Middle East Respiratory Syndrome Coronavirus (MERS-CoV), have led to global epidemics with high morbidity and mortality. Both endemics were caused by zoonotic coronaviruses that belong to the genus Betacoronavirus within Coronaviridae.

Like SARS-CoV and MERS-CoV, the new SARS-CoV-2 belongs to the Betacoronavirus genus. As reported by Zhou et al. (Cell Discovery (2020) 6:14) SARS-CoV-2 shares the highest nucleotide sequence identity with SARS-CoV (79.7%). Specifically, the envelope and nucleocapsid proteins of SARS-CoV-2 are two evolutionarily conserved regions, with sequence identities of 96% and 89.6%, respectively, compared to SARS-CoV. The spike protein was reported to exhibit the lowest sequence conservation (sequence identity of 77%) between SARS-CoV-2 and SARS-CoV, while the spike protein of SARS-CoV-2 only has 31.9% sequence identity with the spike protein of MERS-CoV. The S protein is the most exposed protein and antibody responses against the SARS-CoV S protein have been shown to protect from SARS-CoV infection in a mouse model.

Several national and international research groups are working on the development of new vaccines to prevent and treat diseases, in particular Covid-19, but the development of effective vaccines to prevent or treat infectious diseases, such a vaccine for HIV, or cancer remains a challenge. Thus, there remains a need for new strategies to develop effective therapeutic and/or prophylactic vaccines that can prevent and/or treat infectious diseases, such as Covid-19 or other emerging coronavirus mediated and/or zoonotic diseases or cancer.

### SUMMARY OF THE INVENTION

The AAV capsid consists of 60 individual structural viral proteins (VP), 5 VP1, 5 VP2 and 50 VP3 proteins. Thus, by inserting an immunogenic protein or a portion thereof into the structural proteins VP1, VP2 and particularly VP3, due to the repetitive structure of the capsid, the immunogenic protein or a portion thereof would be presented multiple times at the surface of an intact AAV capsid. This provides a high density of immunogenic protein and mimics the appearance of a virus, such as SARS-CoV-2, with the regular repetitive presentation of viral structural proteins (e.g., the Spike protein) at the surface. This platform technology can also be exploited by inserting targeting molecules, i.e., protein comprising a binding domain (e.g., an antigen-binding domain, such as in an antibody-derived protein or antibody mimetic) that bind as one binding unit of a binding pair to the other binding unit of the binding pair (e.g., an antigen).

The present invention relates to the insertion of a large immunogenic protein or a portion thereof, such as the main antigenic entity of an infectious agent or an immunogenic portion thereof, into surface-exposed positions of AAV capsids in order to re-purpose the AAV vector as a vaccine. In particular, the adeno associated virus (AAV) or the adeno-associated virus-like particle (AAVLP) is converted into a carrier vehicle for immunogenic amino acid sequences of varying length, which are encoded within the capsid VP sequence, and hence into a carrier for a subunit vaccine. These vaccines can be used for immunization to elicit immune responses, including antibody responses, for treating or preventing disease, or alternatively as research tool for generating/eliciting antibodies, due to the exceptionally strong antigenic properties of the vaccine.

An insert at I-587 or I-453 of AAV2 VP2 or any other insertion-tolerating, surface-exposed position within the common sequence shared by all three VPs (VP1, VP2 and VP3), is inserted in each of the 60 building blocks of the AAV capsid and is thus displayed 60 times at the surface of a single AAV particle. As demonstrated herein, surface exposed position at the top of variable region VIII (VR-VIII) and/or variable region IV (VR-IV) surprisingly tolerate large insertions. In case of simultaneous insertion into two insertion sites one at the top of VR-VIII and one at the top of VR-IV (e.g. I-587 and I-453 of AAV2, respectively) an immunogenic sequence is displayed 120 times at the surface of a single AAV particle or alternatively two different immunogenic sequences are displayed 60 times each at the surface of a single AAV particle. The repetitive structure also allows to insert other proteins, such as proteins/target molecules comprising a binding domain (e.g., an antigen-binding domain, such as in an antibody derived protein or an antibody mimetic) at the top of VR-VIII and/or VR-IV to retarget, i.e., redirect the AAV, thereby conferring an altered cell tropism.

In one aspect, the invention relates to an adeno-associated virus (AAV) or a adeno-associated virus-like particle (AAVLP) comprising an insert of about 75-400 amino acids in the viral proteins (VPs) forming a capsid at an insertion site (I) at the top of variable region VIII and/or variable region IV (VR-VIII and/or VR-IV) of the VPs, and wherein the insert is optionally flanked by a linker comprising one or more amino acids on one or both sides, preferably selected from the group consisting of A (Ala), G (Gly), S (Ser), T (Thr), L (Leu) and combinations thereof. The insert may be any protein having the respective length, particularly the insert may be an immunogenic protein or a portion thereof, and/or a protein comprising a binding domain.

In certain embodiments, the invention relates to an adeno-associated virus (AAV) or a adeno-associated virus-like particle (AAVLP) comprising an insert of about 75-400 amino acids (preferably 75-300 amino acids) in the viral proteins (VPs) forming a capsid at an insertion site (I) at the top of variable region VIII and/or variable region IV (VR-VIII and/or VR-IV) of the VPs, wherein the insert is an immunogenic protein or a portion thereof, and wherein the insert is optionally flanked by a linker comprising one or more amino acids on one or both sides, preferably selected from the group consisting of A (Ala), G (Gly), S (Ser), T (Thr), L (Leu) and combinations thereof.

In certain embodiments, the invention relates to an adeno-associated virus (AAV) or a adeno-associated virus-like particle (AAVLP) comprising an insert of about 75-400 amino acids in the viral proteins (VPs) forming a capsid at an insertion site (I) at the top of variable region VIII and/or variable region IV (VR-VIII and/or VR-IV) of the VPs, wherein the insert is a protein comprising a binding domain, and wherein the insert is optionally flanked by a linker comprising one or more amino acids on one or both sides, preferably selected from the group consisting of A (Ala), G (Gly), S (Ser), T (Thr), L (Leu) and combinations thereof. In certain embodiments, the protein comprising a binding domain is a protein comprising a receptor-binding domain, a ligand binding domain or an antigen-binding domain, preferably an antigen-binding domain.

The top of VR-VIII corresponds to amino acids 585 to 592 (I-585 to I-592) of VP1, more specifically to about amino acids 585 to 592 (I-585 to I-592) of VP1 AAV1 having the amino acid sequence of SEQ ID NO: 1, VP1 AAV2 having the amino acid sequence of SEQ ID NO: 2, VP1 AAV3 having the amino acid sequence of SEQ ID NO: 3, VP1 AAV6 having the amino acid sequence of SEQ ID NO: 6, VP1 AAV7 having the amino acid sequence of SEQ ID NO: 7, VP1 AAV8 having the amino acid sequence of SEQ ID NO: 8, VP1 AAV9 having the amino acid sequence of SEQ ID NO: 9 or VP1 AAV10 having the amino acid sequence of SEQ ID NO: 10 or to about amino acids 583 to 589 (I-583 to I-589) of VP1 AAV4 having the amino acid sequence of SEQ ID NO: 4, or to about amino acids 574 to 580 (I-574 to I-580) of VP1 AAVS having the amino acid sequence of SEQ ID NO: 5. Alternatively the top of VR-VIII may be defined as the 8 amino acids downstream of the conserved glutamine corresponding to Q584 of VP1 AAV2 having the amino acid sequence of SEQ ID NO: 2.

The top of VR-IV corresponds to amino acids 450-460 (I-450 to I-460) of VP1, more specifically to about amino acids 450 to 460 (I-450 to I-460) of VP1 AAV1 having the amino acid sequence of SEQ ID NO: 1, VP1 AAV2 having the amino acid sequence of SEQ ID NO: 2, VP1 AAV3 having the amino acid sequence of SEQ ID NO: 3, VP1 AAV6 having the amino acid sequence of SEQ ID NO: 6, VP1 AAV7 having the amino acid sequence of SEQ ID NO: 7, VP1 AAV8 having the amino acid sequence of SEQ ID NO: 8, VP1 AAV9 having the amino acid sequence of SEQ ID NO: 9 or VP1 AAV10 having the amino acid sequence of SEQ ID NO: 10, or to about amino acids 445 to 455 of VP1 AAV4 having the amino acid sequence of SEQ ID NO: 4, or to about amino acids 439 to 449 of VP1 AA5 having the amino acid sequence of SEQ ID NO: 5. Alternatively the top of VR-IV may be defined as from 12 amino acids to 5 amino acids upstream of the conserved phenylalanine corresponding to F462 of VP1 AAV2 having the amino acid sequence of SEQ ID NO: 2.

Preferably, the AAV or AAVLP is derived from AAV serotype 1 (AAV1), 2 (AAV2), 8 (AAV8) or 9 (AAV9). In one embodiment the AAV or the AAVLP is derived from AAV2 and the insertion site is between two amino acids corresponding to amino acid position 587 and 588 (AAV2 1-587) or 588 and 589 (AAV2 I-588) and/or 452 and 453 (AAV2 I-452), 453 and 454 (AAV2 I-453) or 454 and 455 (AAV2 I-454) of AAV2 VP1 having the amino acid sequence of SEQ ID NO: 2, preferably AAV2 I-587 or AAV2 I-588 or AAV2 I-453, more preferably AAV2 I-587 or AAV2 I-588.

In another embodiment the AAV or AAVLP is derived from AAV1 and the insertion site is between two amino acids corresponding to amino acid position 587 and 588 (AAV1 I-587), 588 and 589 (AAV1 I-588) or 589 and 590 (AAV1 I-589) and/or 454 and 455 (AAV1 I-454), 455 and 456 (AAV1 I-455) or 456 and 457 (AAV1 I-456) of AAV1 VP1 having the amino acid sequence of SEQ ID NO: 1. In another embodiment the AAV or AAVLP is derived from AAV8 and the insertion site is between two amino acids corresponding to amino acid position 588 and 589 (AAV8 I-588) or 589 and 590 (AAV8 I-589) or 590 and 591 (AAV8 I-590) and/or 455 and 456 (I-455), 456 and 457 (I-456), or 457 and 458 (I-457) of AAV8 VP1 having the amino acid sequence of SEQ ID NO: 8. In yet another embodiment the AAV or the AAVLP is derived from AAV9 and the insertion site is between two amino acids corresponding to amino acid position 587 and 588 (AAV9 I-587) or 588 and 589 (AAV9 I-588) or 589 and 590 (AAV9 I-589) and/or 454 and 455 (I-454), 455 and 456 (I-455) or 456 and 457 (1-456) of AAV9 VP1 having the amino acid sequence of SEQ ID NO: 9.

In certain embodiment the AAV or the AAVLP comprises an insert of about 75-300 amino acids, preferably an insert of about 75-260 amino acids in the viral proteins, more preferably an insert of about 75-250 amino acids, even more preferably an insert of about 80-220 amino acids. The linker if present may comprise 1 to 7 amino acids, preferably 1-3 amino acids, more preferably 3 amino acids at the N-terminal side and/or 1-3 amino acids, preferably 2-3 amino acids at the C-terminal side of the immunogenic protein or the portion thereof.

In certain embodiments the AAV or AAVLP is an AAV comprising an ITR-flanked genome and is preferably infectious. The ITR-flanked genome may comprise a transgene, such as encoding a further immunogenic protein or a portion thereof. In certain other embodiments the AAV or AAVLP is an AAVLP not comprising a genome that is flanked by ITRs. In some embodiments the insert is an immunogenic protein or a portion thereof and the immunogenic protein or a portion thereof is inserted at the top of VR-VIII and at the top of VR-IV and wherein the immunogenic protein or a portion thereof inserted at the top of VR-VIII and the immunogenic protein or a portion thereof inserted at the top of VR-IV are the same or different; and/or the AAV or AAVLP is formed by 2 or more viral proteins comprising different inserts of a least about 75-400 amino acids (preferably about 75-300 amino acids), wherein the different inserts are each an immunogenic protein or a portion thereof, either an immunogenic protein or an immunogenic portion from a different protein or a different immunogenic portion from the same protein.

AAV VPs forming the capsid may be VP1, VP2 and VP3, preferably at a ratio of 1:1:10. Capsids may also be formed by VP1 and VP3 only or VP3 only. Thus, the AAV VPs forming the capsid may also be VP1 and VP3 or may be VP3. In all variants the AAV or AAVLP preferably has a capsid of about 60 VPs.

In certain embodiments, the AAV or AAVLP according to the invention is immunogenic for the inserted immunogenic protein or the portion thereof. In certain embodiments, the immunogenic protein or the portion thereof may be a viral, a bacterial or a parasitic protein or a portion thereof. In case of a viral protein the immunogenic protein or the portion thereof is not an AAV protein or a portion thereof. In one embodiment the immunogenic protein or the portion thereof is a portion of coronavirus spike (S) protein, such as a portion of SARS-CoV-2 S protein. The portion of the coronavirus S protein may comprise the coronavirus S protein receptor binding domain (RBD) or a portion thereof, such as the SARS-CoV-2 S protein receptor binding domain (RBD) or a portion thereof, preferably a portion comprising the receptor binding motif (RBM). In one embodiment the portion of the SARS-CoV S protein comprises an amino acid sequence of SEQ ID NOs: 11, 12, 31, 32, 33, 34, 35, 36, 37, 38, 39, 40, 41, 42, 43, 44, 45, 46, 47, 48, 49, 50 or 69, preferably of SEQ ID NOs: 11, 12, 34, 35, 36, 37, 38, 42 or 69 or an amino acid sequence having at least 90% identity with the amino acid sequence of SEQ ID NOs: 11, 12, 31, 32, 33, 34, 35, 36, 37, 38, 39, 40, 41, 42, 43, 44, 45, 46, 47, 48, 49, 50 or 69, preferably of SEQ ID NOs: 11, 12, 34, 35, 36, 37, 38, 42 or 69. In other embodiments, the immunogenic protein or the portion thereof is a tumor antigen.

In certain embodiments the insert is a protein comprising a binding domain, such as an antigen-binding domain (e.g., a single-domain antibody (sdAb), a single chain variable fragment (scFv) or an antibody mimetic, such as an anticalin). The AAV or AAVLP is preferably an AAV comprising an ITR-flanked genome and is infectious, wherein the ITR-flanked genome may comprise a transgene.

In a further aspect the invention relates to a pharmaceutical composition comprising the AAV or AAVLP according to the invention, and preferably further at least one pharmaceutically acceptable excipient.

In yet a further aspect the invention relates to the AAV or AAVLP of the invention or the pharmaceutical composition of the invention for use in therapy.

In yet a further aspect the invention relates to the AAV or AAVLP or the pharmaceutical composition according to the invention, wherein the insert is an immunogenic protein or a portion thereof for use as a vaccine.

In yet a further aspect the invention relates to the AAV or AAVLP or the pharmaceutical composition according to the invention for use in the treatment or the prevention of a disease induced by a virus, a bacterium or a parasite, wherein the insert is an immunogenic protein or the portion thereof of said virus, bacterium or parasite, respectively. In one embodiment the disease is a coronavirus respiratory syndrome and the immunogenic protein or a portion thereof is the portion of a coronavirus spike (S) protein. In certain embodiments, the disease is coronavirus disease 2019 (COVID-19) and the immunogenic protein or the portion thereof is a portion of the SARS-CoV-2 spike (S) protein. In a specific aspect, the immunogenic protein or the portion thereof in the AAV or AAVLP according to the invention comprises a portion of the SARS-CoV-2 spike (S) protein and the AAV or AAVLP is for use in inducing an immune response against SARS-CoV-2. In certain embodiments of the AAV or AAVLP for use according to the invention the portion of the SARS-CoV-2 spike (S) protein, preferably wherein the portion of the SARS-CoV-2 S protein comprises an amino acid sequence of SEQ ID NOs: 11, 12, 31, 32, 33, 34, 35, 36, 37, 38, 39, 40, 41, 42, 43, 44, 45, 46, 47, 48, 49, 50 or 69, preferably of SEQ ID NOs: 11, 12, 34, 35, 36, 37, 38, 42 or 69 or an amino acid sequence having at least 90% sequence identity with the amino acid sequence of SEQ ID NO: 11, 12, 31, 32, 33, 34, 35, 36, 37, 38, 39, 40, 41, 42, 43, 44, 45, 46, 47, 48, 49, 50 or 69, preferably of SEQ ID NOs: 11, 12, 34, 35, 36, 37, 38, 42 or 69. The person skilled in the art will understand that the coronavirus spike (S) protein or a portion thereof is a viral entry protein binding to a cellular receptor and is therefore also a binding protein comprising a binding domain, more specifically a receptor-binding domain. The S protein binds to the cellular receptor ACE-2. Thus, the insert may also be an immunogenic protein or a portion thereof and a protein comprising a binding domain.

In yet a further aspect, the invention relates to the AAV or AAVLP or the pharmaceutical composition according to the invention for use in the treatment or the prevention of cancer and the immunogenic protein or the portion thereof is a tumor antigen or portion thereof. The person skilled in the art will understand that certain viral immunogenic proteins may also act as a tumor antigen, such as HCV or HPV derived antigens.

In yet a further aspect, the invention relates to the AAV or AAVLP of the invention or the pharmaceutical composition according to the invention for use in gene therapy, wherein the insert is a protein comprising a binding domain, such as an antigen-binding domain (e.g., a single-domain antibody (sdAb), a single chain variable fragment (scFv) or an antibody mimetic). The AAV or AAVLP is preferably an AAV comprising an ITR-flanked genome and is infectious, more preferably wherein the ITR-flanked genome comprises a transgene.

The AAV or AAVLP for use according to the invention may be administered via the intranasal mucosal, sublingual, oral, buccal, intravenous, intramuscular, intraperitoneal or subcutaneous route. In one embodiment, the AAV or AAVLP may be administered by inhalation via the intranasal, oral and/or mucosal route.

In yet another aspect, the invention relates to a method for producing an AAV or an AAVLP comprising the steps of (i) preparing a cell comprising at least one DNA sequence comprising a cap gene and a rep gene, at least one DNA sequence comprising adenoviral helper sequences and optionally at least one DNA sequence comprising an ITR-flanked genome; wherein the cap gene encodes a protein comprising an insert of about 75-400 amino acids (preferably about 75-300 amino acids) in the viral proteins (VPs) forming the capsid at an insertion site (I) at the top of variable region VIII and/or variable region IV (VR-VIII and/or VR-IV) of the VPs, and wherein the insert is optionally flanked by a linker comprising one or more amino acids on both sides, preferably selected from the group consisting of A (Ala), G (Gly), S (Ser), T (Thr), L (Leu) and combinations thereof; (ii) cultivating the cells under conditions allowing the production of the AAV or the AAVLP; and (iii) purifying the AAV or the AAVLP. In certain embodiments the method is for producing a pharmaceutical composition comprising said AAV or AAVLP further comprising a step of (iv) adding at least one pharmaceutically acceptable excipient to formulate the AAV or the AAVLP into a pharmaceutical composition. The insert may be any protein having the respective length, particularly the insert may be an immunogenic protein or a portion thereof, and/or a protein comprising a binding domain.

The ITR-flanked genome may further comprise a transgene. Particularly where the insert is an immunogenic protein or a portion thereof, the ITR-flanked genome may further comprise a transgene encoding a further immunogenic protein or a portion thereof. In certain other embodiments the AAV or AAVLP is an AAVLP not comprising a genome that is flanked by ITRs. AAV VPs forming the capsid may be VP1, VP2 and VP3, preferably at a ratio of 1:1:10. Capsids may also be formed by VP1 and VP3 only or VP3. Thus, the AAV VPs forming the capsid may also be VP1 and VP3 or may be VP3. In all variants the AAV or AAVLP preferably has a capsid of about 60 VPs. The immunogenic proteins or a portion thereof and/or the protein comprising a binding domain and the insertions sites may be as disclosed herein for the AAV or AAVLP according to the invention.

### SHORT DESCRIPTION OF THE FIGURES

FIG. 1: Structural models of AAV capsid and AAV viral protein and schematic illustration of the cap ORF. (A) Schematic illustration of the ORF reading frame, including VP1, VP2 and VP3. The location of the representative insertion site I-587 comprising an insert is illustrated at the bottom. (B-C) Comparative structural modelling using Robetta from known structures using Robetta (https://robetta.bakerlab.org/) of (B) VP3 of AAV2 comprising an S protein domain of SARS-CoV-2 as insert at I-587 (HtW2_S1.1) based on (C) the published structure of AAV2 WT (database: protein data bank (PDB)/ID: 6ih9) processed using Chimera software (https://www.cgl.ucsf.edu/chimera) in the same orientation. The AAV VP part is coloured in grey and the S protein part is coloured in black. (D-E) Comparative structural modelling from sequence information using RoseTTAFold (https://robetta.bakerlab.org) of (D) VP3 of AAV2 comprising an S protein domain of SARS-CoV-2 as insert at I-587 (HtW2_S1.1) compared to (E) the published structure of AAV2 WT (database: protein data bank (PDB)/ID: 6ih9) processed using Chimera software (3) in the same orientation. The AAV VP part is coloured in grey and the S protein part is coloured in black. (F-G) Comparative structural modelling based on sequence information of the corresponding 60-mer capsid structure of HtW2_S1.1 using RoseTTAFold shown from two distinct angles (F) and (G), respectively. The AAV VP part is coloured in grey and the S protein part is coloured in black. Accordingly, the large >200 amino acid S protein insertion did not compromise the principle gross capsid structure. Scale bars in (F, G) mark 100 Ångström.

FIG. 2: AAVx affinity purification chromatography of AAV vectors. (A-C) Chromatograms showing the elution of (A) AAV2 WT particles and (B) HtW2_S1.1 particles produced in the presence of the pTransgene plasmid carrying an ITR-flanked sc-CMV-eGFP expression cassette (=full AAV particles) as well as (C) HtW2_S1.1 particles produced in the absence of a pTransgene plasmid (=empty AAV particles; AAVLP). HtW2_S1.1 full and empty particles bind to the AAVx affinity purification column and elute at a similar but slightly delayed time after initiation of the elution process (indicated by the ml values of elution buffer on the x-axis).

FIG. 3: Transduction assay of AAV vectors in HeLa cells. (A) Representative brightfield and epifluorescence images from HeLa cell cultures at 24 hours (left panels) and 48 hours (right panels) after transduction with MOI 1,000 of AAV-sc-CMV-eGFP packaged with AAV2 WT (upper row) or the novel AAV variant HtW2_S1.1 with an insertion of 202 amino acids comprising part of the SARS-CoV-2 51 spike protein comprising the RBD (SEQ ID NO: 11) flanked by linker amino acids (rows 2-4). The panels in rows 3 and 4 show representative images of HtW2_S1.1-transduced HeLa cell cultures after transduction with MOI 500 and 250 of AAV-sc-CMV-eGFP packaged with HtW2_S1.1, respectively. Scale bars mark 400 pm. (B) Graph showing the fraction of eGFP-positive cells in % as measured with Countess II FL Automated Cell Counter in HeLa cell cultures at 48 hours after transduction with MOI 1,000 of AAV-sc-CMV-eGFP packaged with AAV2 WT (upper panels) or with MOI 1,000, 500, and 250 of AAV-sc-CMV-eGFP packaged with the novel AAV variant HtW2_S1.1 with an insertion of 202 amino acids comprising part of the SARS-CoV-2 51 spike RBD flanked by linker amino acids. Despite large insertion of more than 200 amino acids, HtW2_S1.1 still retained the ability to infect and transduce human cells even at very low MOI 250.

FIG. 4: Transduction assay of HtW2_S1.1 vectors in HeLa cells. (A) Representative epifluorescence images from native (left column, −ACE2) or ACE2-transfected (right column, +ACE2) HeLa cell cultures at 24 hours (left panels) and 48 hours (right panels) after transduction with MOI 250 (upper row) or MOI 500 (bottom row) of AAV-sc-CMV-eGFP packaged with the novel AAV variant HtW2_S1.1 with an insertion of 202 amino acids comprising part of the SARS-CoV-2 S1 spike protein comprising the RBD (SEQ ID NO: 11). Scale bars mark 400 μm. (B) Graph showing the fraction of eGFP-positive cells in % as measured with Countess II FL Automated Cell Counter in native or ACE2-transfected HeLa cell cultures at 48 hours after transduction with MOI 250 and 500 of AAV-sc-CMV-eGFP packaged with the novel AAV variant HtW2_S1.1 with an insertion of 202 amino acids comprising part of the SARS-CoV-2 S1 spike RBD flanked by linker amino acids. 1-way ANOVA, S̆idák's multiple comparisons test: **, p<0.01; ***, p<0.001.

FIG. 5: Transduction assay of HtW2_S1.2 vectors in HEK293T cells. (A) Representative epifluorescence images from native (left column, −ACE2) or stable ACE2-overexpressing (right column, +ACE2) HEK293T cell cultures at 48 hours (right panels) after transduction with MOI 250 (upper row), MOI 500 (middle row) or MOI 1000 (bottom row) of AAV-sc-CMV-eGFP packaged with the novel AAV variant HtW2_S1.2 with an insertion of 211 amino acids comprising part of the SARS-CoV-2 S1 spike protein (SEQ ID NO: 69) flanked by linker amino acids. Scale bars mark 200 μm. (B) Graph showing the fraction of eGFP-positive cells in % as measured with Countess II FL Automated Cell Counter in native or stable ACE2-overexpressing HEK293T cell cultures at 48 hours after transduction with MOI 250, 500 and 1000 of AAV-sc-CMV-eGFP packaged with the novel AAV variant HtW2_S1.2 with an insertion of 206 amino acids comprising part of the SARS-CoV-2 S1 spike RBD flanked by linker amino acids. 1-way ANOVA, S̆idák's multiple comparisons test: ***, p<0.001; ****, p<0.0001.

FIG. 6: Humoral response to HtW capsids. (A) Immunization scheme in rabbits (12 weeks old female) (B) Immunogenicity of HtW capsids was assessed in serum from rabbits immunized with indicated AAV empty capsids by ELISA. Shown are the IgG endpoint titers against SARS-CoV-2 wild type RBD. Rabbits were immunized with wildtype AAV empty capsids (AAV2 WT, AAV9 WT) or HtW empty capsids (HtW2_S1.1, HtW2_S1.2 or HtW9_S1.1). (C) Rabbit sera collected 10 days after the first (Bleed1), second (Bleed2) and third (Bleed3) booster injection with the empty AAV capsids were analysed by ELISA using SARS-Cov-2 RBD as antigen. Shown are the endpoint titers of SARS-CoV-2 RBD specific IgG and IgM antibodies.

FIG. 7: Analysis of rabbit sera elicited against HtW capsids using dot blot analysis. (A) Schematic representation of dot blot assays of AAV vectors spotted on polyvinylidene difluoride (PVDF) membranes and stained with indicated antibodies or sera. (B) Pipetting scheme indicating the capsid used for producing each AAV vector and the amount (total vector genomes) of AAV vector spotted on each dot of the blots shown in (C-F). (C) Dot blot labelled with rabbit monoclonal anti-SARS-CoV-2 Spike S1 commercial antibody at 1:500 dilution. (D) Dot blot labelled with a 1:10000 dilution of a serum (αHtW2_S1.1) from a rabbit which was immunized with HtW2_S1.1 empty capsid. (E) Dot blot labeled with a 1:10000 dilution of a serum (aHtW2_S1.2) from a rabbit which was immunized with HtW2_S1.2 empty capsid. (F) Dot blot labelled with a 1:10000 dilution of a serum (αHtW9_S1.1) from a rabbit which was immunized with HtW9_S1.1 empty capsid.

FIG. 8: Dot blot assays of AAV vectors spotted on PVDF membranes and stained with sera from Comirnaty-vaccinated human individuals and Htw9_S1.1-vaccinated rabbits. (A) Pipetting scheme indicating the capsid used for producing each AAV vector and the amount (total vector genomes) of AAV vector spotted on each dot of the blot shown in (B-C). (B) Dot blot labelled with a 1:500 dilution of a serum from a patient collected 1 weeks after the second vaccination with Comirnaty (BNT162b2, Biontech/Pfizer). (C) The same dot blot, which was stripped and re-labelled with a 1:10000 dilution of a serum (αHtW9_S1.1) from a rabbit which was immunized with HtW9_S1.1 empty capsid.

FIG. 9: Neutralization efficiency of sera from HtW-immunized rabbits. (A) Neutralization assay with serum of HtW9_S1.1-immunized rabbits on transduction efficiency in HEK293T cells stably expressing ACE2 (HEK293T+ACE2). HtW2_S1.1 or HtW2_S1.2 vectors with sc-CMV-eGFP genome were pre-incubated at 37° C. with different dilutions (1:1000, 1:5000, 1:10000) of αHtW9_S1.1 serum and used to transduce HEK293T+ACE2 cells at an MOI of 250. Shown are epifluorescence microscopy images 48 h post transduction. (B) 48 h post transduction cells were collected and the fraction of eGFP-positive cells was analysed using a Countess II FL Automated Cell Counter. Shown are the ratios of eGFP positive cells following transduction with HtW2_S1.1 or HtW2_S1.2 pre-incubated with αHtW9_S1.1 at indicated dilutions normalized to the corresponding control transduction with the respective vector but in the absence of the serum.

FIG. 10: Structural models of AAV viral protein (VP) of serotypes AAV2 (A), AAV9 (B), AAV1 (C) and AAV8 (D). (A-D) Comparative structural modelling (https://robetta.bakerlab.orq/) of VP3 of AAV2, AAV9, AAV8 and AAV9 with the top of the variable region VIII corresponding to amino acids 585-592 and variable region IV corresponding to amino acids 450-460 highlighted in black (A) AAV2 WT (PDB 6ih9) amino acids 585-592 (SEQ ID NO: 17) and amino acids 450-460 (SEQ ID NO: 16) marked, (B) AAV9 WT (3ux1) amino acids 585-592 (SEQ ID NO: 19) and amino acids 450-460 (SEQ ID NO: 18) marked, (C) AAV1 WT (6jcr) amino acids 585-592 (SEQ ID NO: 21) and amino acids 450-460 (SEQ ID NO: 20) marked, and (D) AAV8 WT (2qa0) amino acids 585-592 (SEQ ID NO: 23) and amino acids 450-460 (SEQ ID NO: 22) marked, were processed using Chimera software.

FIG. 11: Multiple sequence alignment of Cap sequences of AAV1, AAV2, AAV8 and AAV9 VP1 having the amino acid sequence of SEQ ID NO: 1, SEQ ID NO: 2, SEQ ID NO: 8 and SEQ ID NO: 9, respectively. Sequences were aligned using Clustal O (1.2.4). The shaded region indicates the insertion site in VR-IV (I-450 to I-460) and in VR-VIII (I-585 to I-592).

FIG. 12: Multiple sequence alignment of Cap sequences of AAV1-10 VP1 having the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10, respectively. Sequences were aligned using Clustal O (1.2.4).

FIG. 13: Vector map of pHtW2_S1.1 (AAV2) expressing the AAV2-Rep protein and the AAV2-CAP protein with an insertion of 202 amino acids comprising the SARS-CoV-2 S1 spike protein flanked by linker amino acids (SEQ ID NO: 13).

FIG. 14: Vector map of pHtW2_S1.2 (AAV2) expressing the AAV2-Rep protein and the AAV2-CAP protein with an insertion of 211 amino acids comprising the SARS-CoV-2 S1 spike protein flanked by linker amino acids (SEQ ID NO: 70).

FIG. 15: Vector map of pHtW9_S1.1 (AAV9) expressing the AAV9-Rep protein and the AAV9-CAP protein with an insertion of 202 amino acids comprising the SARS-CoV-2 S1 spike protein flanked by linker amino acids (SEQ ID NO: 14).

FIG. 16: Structural models of AAV capsid and AAV viral protein. (A-B) RoseTTAFold (https://robetta.bakerlab.org/) based de novo prediction of protein structure of VP3 of AAV2 comprising an anti-GFP scFv antibody fragment (SEQ ID NO: 73) as insert at I-587 (AAV2-αGFP) (A). The corresponding predicted 60-mer capsid structure of AAV2-αGFP is shown in (B). The AAV VP part is coloured in grey and the anti-GFP scFv part is coloured in black. Accordingly, the large >200 amino acid anti-GFP scFv did not compromise the principle gross capsid structure. Scale bar in (B) marks 100 Ångström.

### DETAILED DESCRIPTION OF THE INVENTION

The term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. The term “comprising” may be individually replaced by the term “consisting of”. With regard to sequences the terms “having an amino acid sequence of” and “comprising an amino acid of” are used interchangeably and include the embodiment “consisting of the amino acid sequence of”. The term “a” as used herein may include the plural and hence includes, but is not limited, to “one”. The term “about” as used herein refers to +/−10% of the value specified.

The term “expression cassette” as used herein refers to a nucleic acid unit comprising at least one open reading frame (ORF) under the control of regulatory sequences controlling its expression, such as a promoter. Preferably the expression cassette also comprises a transcription termination signal.

The term “protein” is used interchangeably with “amino acid sequence” or “polypeptide” and refers to polymers of amino acids of any length. These terms also include proteins that are post-translationally modified through reactions that include, but are not limited to, glycosylation, acetylation, phosphorylation, glycation or protein processing. Modifications and changes, for example amino acid sequence substitutions, deletions or insertions, can be made in the structure of a polypeptide while the molecule maintains its biological functional activity. For example, certain amino acid sequence substitutions can be made in a polypeptide or its underlying nucleic acid coding sequence and a protein can be obtained with the same properties. The term “protein” typically refers to a sequence with more than 30 or even more amino acids that typically has a secondary and tertiary structure. The term “peptide” means sequences typically with up to 30 amino acids in length. Typically a peptide is characterized by is primary amino acid sequence. The term “immunogenic” as used herein refers to a substance able to cause an antigen specific immune response in the body of a subject receiving the substance. Such an immune response includes a humoral and/or cell-mediated immune response.

The term “immunogenic protein or a portion thereof” as used herein refers to an antigenic or immunogenic protein, e.g., of a pathogen or a tumor cells, such as a structural viral protein or a tumor antigen or an immunogenic portion thereof. An immunogenic portion of an immunogenic protein typically comprises one or more domain(s) of the immunogenic protein, in case of a transmembrane protein typically one or more domain(s) of the ectodomain of the immunogenic protein. However, it is also encompassed by the present invention that the immunogenic portion comprises only an immunogenic part of a domain, such as the receptor binding domain of a viral entry protein or the ligand binding domain of a tumor antigen. The immunogenic protein or a portion thereof as used herein may serve as a subunit vaccine against the pathogen or the tumor cells expressing said immunogenic protein. The immunogenic protein or a portion thereof as used herein may also serve as a vaccine or antigen to elicit or generate antibodies following vaccination, due to the exceptionally strong antigenic properties of the AAV or AAVLP comprising up to 60 viral proteins comprising said immunogenic protein or a portion thereof.

The term “subunit vaccine” as used herein includes only parts of the pathogen (virus, bacteria or parasite) instead of the entire germ and may also be used in the context of a tumor antigen. Because these vaccines contain only the essential antigens and not all the other molecules that make up the germ or the tumor cells, side effects are less common. The pertussis (whooping cough) component of the DTaP vaccine is an example of a subunit vaccine.

The term “domain” as used herein refers to a folded protein structure which has tertiary structure independent of the rest of the protein. Generally, domains are responsible for discrete functional properties of proteins and in many cases may be added or transferred to other proteins without loss of function or immunogenicity.

The term “insert” as used herein refers to an insertion of at least one amino acid or nucleotide into a sequence of amino acids or nucleotides, rather than substitution. In the context of the present invention it is primarily used in the context of amino acid sequences. An insert may be introduced between two amino acids or may replace a stretch of amino acids resulting in an overall elongated amino acid sequence. According to the present invention an insert has at least about 75 amino acids, preferably about 75-400 amino acids, more preferably about 75 to 300 amino acids. The resulting protein is a chimeric protein with the insert being from a different origin than the VPs. Thus, the “insert” in the AAV or AAVLP according to the invention is a protein or polypeptide of about 75-400 amino acids, more preferably of about 75-300 amino acids fused at the N-terminal and the C-terminal end to the viral protein (VP) at the insertion site as specified herein. The insert may be flanked by a linker comprising one or more amino acids on one or both sides.

The term “transgene” refers to a gene which is artificially introduced into the genome of another organism. A transgene may also be referred to as a heterologous gene. In the case of AAV, the transgene is typically placed between the two ITRs of the genome, such as in the pTransgene plasmid.

The “protein comprising a binding domain” as used herein refers to one binding unit of a binding pair (such as a protein comprising an antigen-binding domain specific for a target antigen, e.g., an antibody derived protein or an antibody mimetic). The binding domain may be a receptor-binding domain, a ligand-binding domain or an antigen-binding domain (also referred to as antigen recognition domain). Thus, the protein comprising a binding domain, i.e., the one binding unit of the binding pair determines the tropism of the AAV or AAVLP for a target cell expressing the binding partner (e.g., receptor or ligand or antigen) for the protein comprising a binding domain, i.e., the other binding unit of the binding pair (such as a target antigen binding to an antigen-binding domain specific for the target antigen) on the surface of said target cell. Suitable binding pairs, without being limited thereto are a protein comprising an antigen-binding domain and its antigen (e.g., a single-domain antibody (sdAb), a single chain variable fragment (scFv) and its antigen or an antibody mimetic, such as an anticalin and its antigen), a protein comprising a receptor-binding domain and a receptor (e.g., the coronavirus spike (S) protein and ACE-2 receptor; an antibody Fc-region (e.g., scFc) and an Fc receptor), and a ligand-binding domain and a ligand (e.g., PD-1 and PD-L1).

The term “tropism” or “cell tropism” as used herein refers to the ability of the virus to transduce certain cell types. Thus, AAVs or AAVLPs with varying tropism have the ability to transduce different cell types, e.g., different retinal cell types. The tropism of an AAV or AAVLP may be changed by recombinant techniques (genetic engineering), which results in a retargeted AAV or AAVLP, i.e., an AAV or AAVLP re-directed to specific cell types other than those normally infected by naturally occurring AAV serotypes. While small peptides inserted into surface-exposed positions of AAV capsids displayed at the surface of the AAV particle have been used to change the AAV tropism, according to the present invention this change of tropism may be effected by an insert of about 75-400, preferably 75-300 amino acids in the viral proteins, wherein the insert is a protein comprising a binding domain, such as an antigen binding domain, e.g., in an antibody derived protein, such as a sdAb or a single-chain Fv or an antibody mimetic, such as an anticalin.

### An AAV or AAVLP Comprising an Insert of a Protein or a Portion Thereof in the Viral Proteins

The present invention relates to an adeno-associated virus (AAV) or a adeno-associated virus-like particle (AAVLP) comprising an insert of at least about 75 amino acids (such as about 75 to 400, preferably about 75 to 300 amino acids) in the viral proteins (VPs) forming a capsid at an insertion site (I) at the top of variable region VIII and/or variable region IV (VR-VIII and/or VR-IV) of the VPs, and wherein the insert is optionally flanked by a linker comprising one or more amino acids on one or both sides, preferably selected from the group consisting of A (Ala), G (Gly), S (Ser), T (Thr), L (Leu) and combinations thereof. The insert may be any protein or a portion thereof. In certain embodiments the insert is (a) an immunogenic protein or a portion thereof, and/or (b) a protein comprising a binding domain. The insert inserted at the top of VR-VIII and the insert inserted at the top of VR-IV may be the same or different. In certain embodiments, the first insert inserted at the top of VR-VIII and the second insert inserted at the top of VR-IV are different. In other or additional embodiments, the AAV or AAVLP according to the invention may also be formed by two or more viral proteins comprising different inserts of a least about 75-400 amino acids. Wherein the insert in each of said two or more viral proteins comprising an insert is a protein or a portion thereof, preferably selected from the group consisting of (a) an immunogenic protein or a portion thereof and (b) a protein comprising a binding domain. In preferred embodiments, each of the viral proteins (VPs) forming a capsid comprise an insert at an insertion site at the top of VR-VIII or VR-IV of the VPs.

Thus, in certain embodiments, the present invention relates to an adeno-associated virus (AAV) or a adeno-associated virus-like particle (AAVLP) comprising an insert of at least about 75 amino acids (such as about 75-400, preferably about 75 to 300 amino acids) in the viral proteins (VPs) forming a capsid at an insertion site (I) at the top of variable region VIII and/or variable region IV (VR-VIII and/or VR-IV) of the VPs, wherein the insert is an immunogenic protein or a portion thereof, and wherein the insert is optionally flanked by a linker comprising one or more amino acids on one or both sides, preferably selected from the group consisting of A (Ala), G (Gly), S (Ser), T (Thr), L (Leu) and combinations thereof. The immunogenic protein or a portion thereof inserted at the top of VR-VIII and the immunogenic protein or a portion thereof inserted at the top of VR-IV may be the same or different, wherein different may be an immunogenic protein or an immunogenic portion from a different protein or a different immunogenic portion from the same protein. Preferably, the immunogenic protein or the immunogenic portions from different proteins are in case of a pathogen from the same pathogen (such as the same bacteria, the same virus or the same parasite). In certain embodiments, the immunogenic protein or a portion thereof inserted at the top of VR-VIII and the immunogenic protein or a portion thereof inserted at the top of VR-IV are different (such as an immunogenic protein or an immunogenic portion from a different protein or a different immunogenic portion from the same protein). In other or additional embodiments, the AAV or AAVLP according to the invention may also be formed by 2 or more viral proteins comprising different inserts of a least about 75-400 amino acids, wherein the different inserts are each an immunogenic protein or a portion thereof, either an immunogenic protein or an immunogenic portion from a different protein or a different immunogenic portion from the same protein. In preferred embodiments, each of the viral proteins (VPs) forming a capsid comprise an insert at an insertion site at the top of VR-VIII or VR-IV of the VPs.

The present invention also relates to an adeno-associated virus (AAV) or a adeno-associated virus-like particle (AAVLP) comprising an insert of at least about 75 amino acids (such as about 75-400, preferably 75 to 300 amino acids) in the viral proteins (VPs) forming a capsid at an insertion site (I) at the top of variable region VIII and/or variable region IV (VR-VIII and/or VR-IV) of the VPs, wherein the insert is protein comprising a binding domain, and wherein the insert is optionally flanked by a linker comprising one or more amino acids on one or both sides, preferably selected from the group consisting of A (Ala), G (Gly), S (Ser), T (Thr), L (Leu) and combinations thereof. In certain embodiments, the protein comprising a binding domain is a protein comprising a receptor-binding domain, a ligand binding domain or an antigen-binding domain, preferably an antigen-binding domain. The protein comprising a binding domain inserted at the top of VR-VIII and the protein comprising a binding domain inserted at the top of VR-IV may be the same or different, wherein different means proteins comprising binding domains with different binding specificities (e.g., different antigen-binding specificities). Alternatively, only one of the inserts may be a protein comprising a binding domain. In other or additional embodiments, the AAV or AAVLP according to the invention may also be formed by two or more viral proteins comprising different inserts of a least about 75-400 amino acids. The different inserts each may be a protein comprising a binding domain, preferably with different binding specificities, or at least one of the inserts is a protein comprising a binding domain. In preferred embodiments, each of the viral proteins (VPs) forming a capsid comprise an insert at an insertion site (i) at the top of VR-VIII or VR-IV of the VPs.

The AAV VP3 region (and hence also VP1 and VP2) contains highly conserved regions that are common to all serotypes, a core eight-stranded β-barrel motif (βB-βI) and a small α-helix. The loop regions inserted between the β-strands consist of the distinctive HI loop between β-strands H and I, the DE loop between β-strands D and E, and nine variable regions (VRs), which form the top of the loops. These VRs are found on the capsid surface and can be associated with specific functional roles in the AAV life cycle including receptor binding, transduction and antigenic specificity (Drouin and Agbandje-McKenna, Future Virol., 2013, 8(12):1183-1199).

At least 13 distinct human and non-human primate AAV serotypes (AAV1-AAV13) have been sequenced. The AAVs are classified into six genetic groups (clades A-f) and two clonal isolates (AAV4 and AAV5) based on antigenic reactivity and sequence comparison. Comparing the AAV serotypes they have only approximately 65-99% sequence identity, but a high structural identity of 95-99% (percentage of superposable Cα position) (Drouin and Agbandje-McKenna, Future Virol., 2013, 8(12):1183-1199).

Due to the high structural identity, the top of VR-VIII or VR-IV can be easily determined for an AAV VP1 sequence by the person skilled in the art. Structural data and 3D views of the structural proteins of AAV are available from most serotypes in protein data bank, an open access database for the three-dimensional structural data of large biological molecules (https://www.rcsb.org/). For example, the structure for AAV 1, 2, 5, 8 and 9 may be found under the following ID numbers 6ih9 (AAV2), 6jcr (AAV1), 6jct (AAV5), 2qa0 (AAV8), 3ux1 (AAV9), Alternatively the 3D structure of a given sequence may be generated using comparative structural modelling using protein structure prediction services based on known structures, using, e.g., the Robetta protein structure prediction service (https://robetta.bakerlab.orq/), or the more recently available protein structure prediction from sequence information improved deep learning based modelling service, e.g., the RoseTTAFold protein structure prediction service (https://robetta.bakerlab.orq/). The top of VR-VIII and VR-IV may be identified by identifying the amino acid at the outermost tip of VR-VIII and VR-IV and about 2-5 amino acids upstream and downstream thereof.

The AAV or AAVLP according to the invention may be derived from any AAV serotype. Non-limiting examples of AAVs include AAV type 1 (AAV-1), AAV type 2 (AAV-2), AAV type 3 (AAV-3), AAV type 3B (AAV-3B), AAV type 4 (AAV-4), AAV type 5 (AAV-5), AAV type 6 (AAV-6), AAV type 7 (AAV-7), AAV type 8 (AAV-8), AAV type 9 (AAV9), AAV type 10 (AAV10), AAV11, AAV12, AAV13, rh10, avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc. Preferably, the AAV or AAVLP is derived from AAV serotype 1 (AAV1), 2 (AAV2), 3 (AAV3), 4 (AAV4), 5 (AAV5), 6 (AAV6), 7 (AAV7) 8 (AAV8), 9 (AAV9) or 10 (AAV10), more preferably 1 (AAV1), 2 (AAV2), 8 (AAV8) or 9 (AAV9). The corresponding insertion sites in these AAVs can be transferred from the specific insertion sites disclosed herein for AAV1 to AAV10 and/or the respective 3D structure as described above.

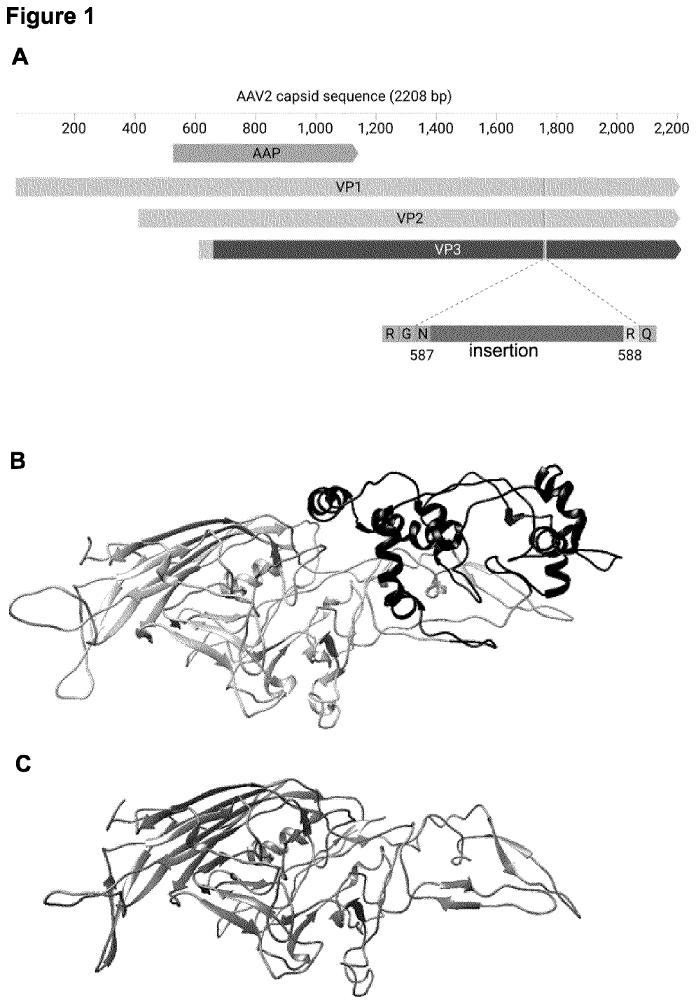

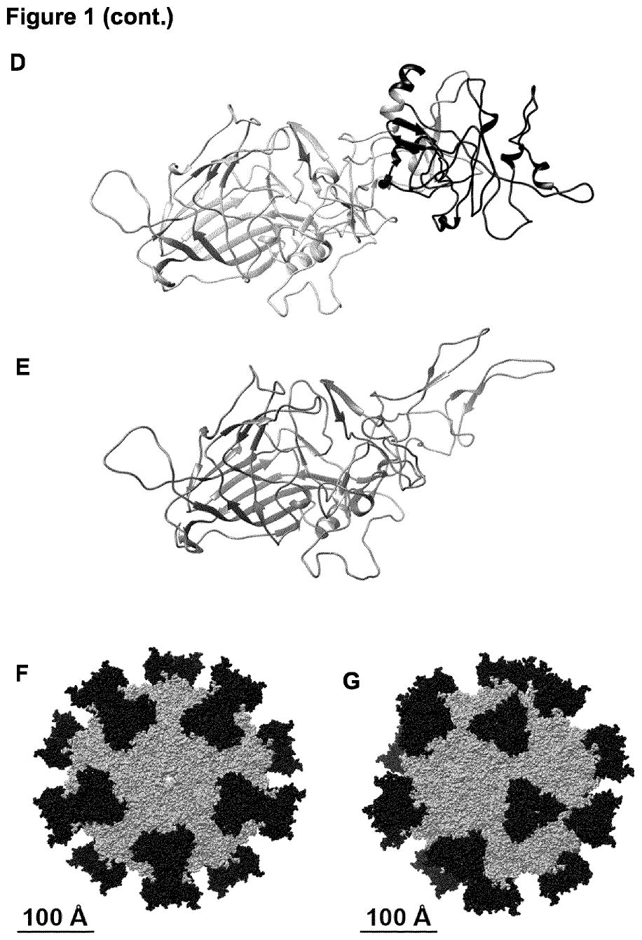

The term “viral proteins” abbreviated as VPs as used herein refers to the viral proteins VP1, VP2 and VP3 that interact to form a capsid and are therefore the AAV structural proteins. The viral proteins may also be referred to as capsid proteins. The AAV capsid is a non-enveloped, icosahedral 60-mer of three repeating monomers, VP1, VP2 and VP3 ata 1:1:10 stoichiometry. The icosahedral capsid has a diameter of approximately 260 Å. The three structural proteins VP1, VP2 and VP3 are produced from the cap ORF comprising the single cap gene using the P40 promoter by alternative splicing and the usage of an alternative non-canonical ACG translation start codon for VP2, resulting in three distinct protein products that share C-terminal identity the length of VP3 (FIG. 1A). VP3-only or capsids consisting of VP1 and VP3 can be assembled and packaged with a genome. However, VP3-only particles are non-infectious because of the absence of a PLA2 domain encoded in the VP1-unique region of the cap ORF.

The person skilled in the art will understand that specific insertion sites are provided as corresponding to an amino acid position of a VP1 protein having a certain sequence provided, which also specifies the insertion site in the common sequence with VP2 and particularly VP3. The VP3 start corresponds to amino acid position M203 of VP1 for AAV1 (SEQ ID NO: 1), AAV2 (SEQ ID NO: 2), AAV3 (SEQ ID NO: 3), AAV6 (SEQ ID NO: 6) and AAV9 (SEQ ID NO: 9), corresponds to M204 of VP1 for AAV8 (SEQ ID NO: 8) and AAV10 (SEQ ID NO: 10) and corresponds to M197 of VP1 for AAV4 (SEQ ID NO: 4) and M193 of VP1 for AAV5 (SEQ ID NO: 5). We note that for AAV7 the start for VP3 corresponds to position 204 of VP1 (SEQ ID NO: 7) (AAV7 VP3 uses the unusual GTG start codon, see paragraph

in EP 1 456 419 B1). AAV3, as used herein, comprises AAV3A and AAV3B. The amino sequence of SEQ ID NO: 3 provided for the cap protein of AAV3 relates to AAV3B. However, AAV3A may likewise be used in the context of the present invention and the person skilled in the art will be able to identify the respective integration sites.

The term “insertion site” as used herein refers to a position in an amino acid sequence defined by amino acid positions and includes that a polypeptide insert may be introduced between two amino acids adjacent to each other. However, the person skilled in the art will understand that a polypeptide insert may also be introduced between two amino acids that are not directly adjacent to each other, resulting in a replacement of a short stretch of amino acids, such as 2, 3, 4, 5 or more amino acids, preferably 2, 3 or 4 amino acids within the top of VR-VIII or VR-IV. Preferably the insert is inserted between two adjacent amino acids. As used herein the insertion site (I) may be referred to as, e.g., I-587 or AAV2 I-587, which means an insertion site between two amino acids corresponding to amino acid position 587 and 588 or between amino acid position 578 and a following amino acid within the top of VR-VIII as defined by the respective amino acid positions.

The term “adeno-associated virus” abbreviated as AAV as used herein refers to an assembled capsid of native or recombinant AAV packaged with a DNA genome. Native (or naturally occurring) AAV is a single-stranded DNA parvovirus. In the context of the present invention the AAV is a recombinant AAV (rAAV), i.e., a genetically engineered AAV, including comprising modified capsid proteins and/or comprising a heterologous polynucleotide sequence in its viral genome. rAAV can be produced as particles containing a single-strand DNA (ssDNA) or double strand DNA (dsDNA) genome. The rAAV packaged with the ssDNA or dsDNA genome may also be referred to as full AAV (or rAAV) particle. The ssDNA genome is characterized by two functional inverted terminal repeat (ITR) sequences flanking the genome at the 5′ (5′ ITR) and 3′ (3′ ITR) end and the dsDNA genome is characterized by one functional ITR (either at the 5′ or 3′ end) and a second mutated ITR with a deletion covering the terminal resolution site (trs), resulting in a double-stranded or self-complementary DNA genome (scDNA). Both, a single ssDNA genome and a dsDNA genome are referred to as ITR-flanked genome herein. Typically, such AAV (or rAAV) comprising an ITR-flanked genome are infectious, however, as explained above in case of a VP3-only capsid the AAV (or rAAV) may also be non-infectious.

The term “adeno-associated virus-like particle” abbreviated as AAVLP as used herein refers to an assembled capsid that is not packaged with an ITR-flanked genome (ssDNA or dsDNA genome). The AAVLP may therefore also be referred to as empty AAV particle. The AAVLP may package some DNA, but does not comprise a genome, wherein the genome (ssDNA or dsDNA) is characterized by a 5′ and a 3′ ITR. Thus, AAVLPs are non-infectious. In the Examples, AAVLPs are produced in the absence of the pTransgene plasmid carrying the ITR-flanked expression cassette.

The term “inverted terminal repeat” (ITR) as used herein refers includes any viral terminal repeat or synthetic sequence that forms a hairpin structure and functions as an inverted terminal repeat (i.e., mediates the desired functions, such as replication, virus packaging, integration and/or provirus rescue). The ITR can be an AAV ITR or a non-AAV ITR sequence such as those of other parvoviruses (e.g., canine parvovirus (CPV), mouse parvovirus (MVM), human parvovirus B-19) or any other suitable virus sequence. For example the SV40 hairpin that serves as the origin of SV40 replication can be used as an ITR. An ITR sequence can further be modified by truncation, substitution, deletion, insertion and/or addition. Further, the ITR can be partially or completely synthetic, such as the “double-D sequence” as described in U.S. Pat. No. 5,478,745. An “AAV terminal repeat” or “AAV ITR” may be from any AAV, including but not limited to serotypes 1, 2, 3, 3B, 4, 5, 6, 7, 8, 9, 10, 11, 12 or 13, or any other AAV. An AAV terminal repeat need not have the native terminal repeat sequence (e.g., a native AAV ITR sequence may be altered by insertion, deletion, truncation and/or missense mutations), as long as the terminal repeat mediates the desired functions, e.g., replication, virus packaging, integration, and/or provirus rescue, and the like. to a region of the AAV genome comprising all elements involved in genome rescue, replication and packaging, which is about 145 nucleotides long. Thus, ITR as used herein refers to the cis-elements needed for at least packing of the genome.

The term “genome” as used herein in the context of an AAV refers to a DNA sequence comprising a 5′ and a 3′ inverted terminal repeat (ITR). Typically, the genome is a single stranded DNA genome (ssDNA genome). Using a mutated ITR, however, the genome may also be a double stranded DNA genome (dsDNA genome) or self-complementary DNA genome, which is also packaged. Naturally, the AAV genome comprises the rep and the cap gene. In the context of a recombinant AAV, the genome often encodes a transgene (comprises an expression cassette encoding a transgene) flanked by ITRs, and the rep and the cap gene are provided in trans. As used herein, the genome may comprise coding sequences, such as encoding a transgene or the rep and/or cap gene. Alternatively, the genome may comprise non-coding sequences with immune-stimulating effect, such as CpG motifs. Also, the ssDNA itself may have an immune-stimulating effect.

AAV capsids were known to tolerate insertion of small peptides in specific surface-exposed positions without losing their structural integrity and principle function. The most commonly used insertion sites in AAV2 are I-587 (e.g., insertion between the amino acid residue asparagine (N) 587 and arginine (R) 588 of AAV2 VP1) and I-453 (e.g., insertion between the amino acid residue glycine (G) 453 and threonine (T) 454 of AAV2 VP1). Such engineering of the AAV capsid had been explored for changing the AAV tropism and for re-directing the AAVs to specific cell types other than those normally infected by naturally occurring AAV serotypes (Buning, H and Srivastava, A., Mol Ther Methods Clin Dev., 2019, 12: 248-265).

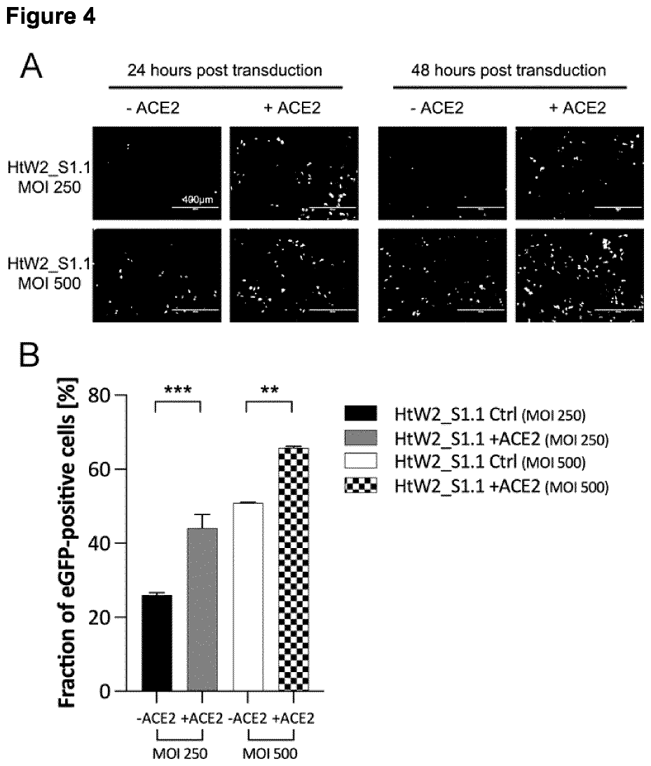

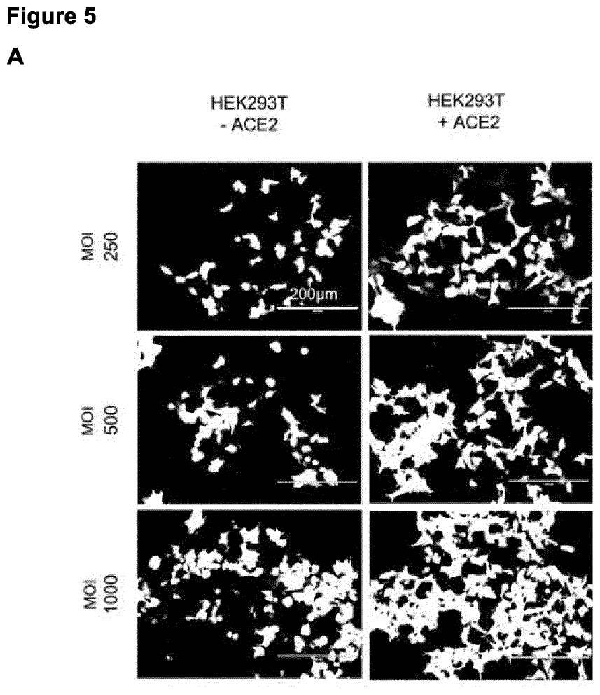

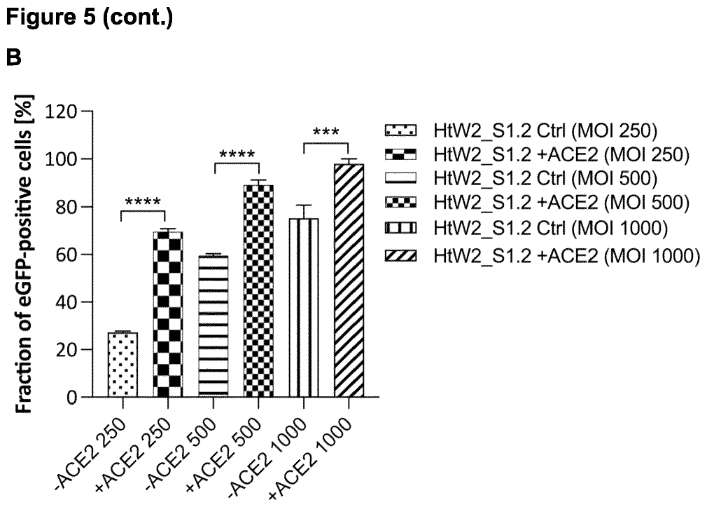

The present invention expands these previous applications by insertion of a large immunogenic protein or a portion thereof comprising about 75 or more amino acids (e.g., 75-400 amino acids or 75-300 amino acids), such as the main antigenic entity of an infectious agent or the immunogenic domain or portion thereof, into surface-exposed positions of AAV capsids in order to re-purpose the AAV vector as a vaccine. In particular, the adeno associated virus (AAV) or the adeno-associated virus-like particle (AAVLP) is converted into a carrier vehicle for immunogenic amino acid sequences of varying length, which are encoded within the capsid VP sequence, and hence into a carrier for a subunit vaccine. By such insertion at, e.g., I-587 or I-453 or any other insertion-tolerating, surface-exposed position within the common part shared by all three VPs (VP1, VP2 and VP3), the immunogenic protein or a portion thereof is inserted in each of the 60 building blocks of the AAV capsid and is thus displayed 60 times at the surface of a single AAV particle. As demonstrated herein, surface exposed position that surprisingly tolerate large insertions are at the top of variable region VIII (VR-VIII) and/or variable region IV (VR-IV). In case of simultaneous insertion into two insertion sites one at the top of VR-VIII and one at the top of VR-IV (e.g. I-587 and I-453 of AAV2, respectively) the immunogenic protein or a portion thereof is displayed 120 times at the surface of a single AAV particle. Alternatively, in case of simultaneous insertion of one immunogenic protein or a portion thereof into the insertion site at the top of VR-VIII and another immunogenic protein or a portion thereof at the top of VR-IV, the two immunogenic proteins or a portion thereof are displayed 60 times each at the surface of a single AAV particle. Preferably, each VP comprises an insert at the top of VR-VIII or VR-IV. In case two or more viral proteins comprising different inserts are used, different insertion sites in each viral protein may be used. Insertions at I-587 of AAV2 disrupt the natural heparan sulphate proteoglycan (HSPG) binding site which defines the cellular tropism of AAV2 (Opie et al., J Virol., 2003, 77:6995-7006; Kern et al., J Virol., 2003, 77:11072-11081). Thus, the insert in the AAV or the AAVLP according to the invention may also alter the tropism of the virion (FIGS. 4 and 5) and potentially facilitate optimal exposure of the inserted protein or a portion thereof according to the biological features determined by its sequence, thereby facilitating, for example, the induction of a stronger immune response of an immunogenic sequence.

The present invention further expands these previous applications by insertion of a protein comprising about 75 or more amino acids (e.g., 75-400 amino acids or 75-300 amino acids), into surface-exposed positions of AAV capsids, wherein the protein may be any protein, particularly a protein comprising a binding domain, such as an antigen binding domain (e.g. an sdAb or an scFv or an antibody mimetic such as an anticalin), in order to re-target the AAV vector. In particular, the adeno-associated virus (AAV) or the adeno-associated virus-like particle (AAVLP) is converted into a vehicle with altered cell tropism, with the protein comprising said binding domain being encoded within the capsid VP sequence, and hence into a vehicle for, e.g., gene therapy. By insertion at, e.g., I-587 or I-453 or any other insertion-tolerating, surface-exposed position within the common part shared by all three VPs (VP1, VP2 and VP3), the protein comprising said binding domain is inserted in each of the 60 building blocks of the AAV capsid and is thus displayed 60 times at the surface of a single AAV particle. As demonstrated herein, surface exposed positions that surprisingly tolerate large insertions are at the top of variable region VIII (VR-VIII) and/or variable region IV (VR-IV). In case of simultaneous insertion into two insertion sites, one at the top of VR-VIII and one at the top of VR-IV (e.g. I-587 and I-453 of AAV2, respectively), the protein comprising a binding domain is displayed 120 times at the surface of a single AAV particle. Alternatively, in case of simultaneous insertion of one protein (such as a first protein comprising a binding domain, e.g., a first sdAb or scFv) into the insertion site at the top of VR-VIII and another protein (such as a second protein comprising a binding domain, e.g., a second sdAb or scFv) at the top of VR-IV, the two different proteins comprising a binding domain are displayed 60 times each at the surface of a single AAV particle. Preferably, each VP comprises an insert at the top of VR-VIII or VR-IV. As explained above, the AAV or AAVLP according to the invention may also be formed by two or more viral proteins comprising different inserts of at least about 75-300 amino acids, wherein the first insert is a first protein comprising a binding domain (such as an antigen-binding domain, e.g., a first sdAb or scFv) and the second insert may be a further protein comprising a binding domain (such as a further protein comprising an antigen-binding domain, e.g., a second sdAb or scFv). Alternatively, the second protein may be an immunogenic protein or a portion thereof. The person skilled in the art will further understand that the insert may be a protein comprising more than one binding domains (i.e. a bispecific protein) at the top of VR-VIII and/or VR-IV, preferably at the top of VR-VIII or VR-IV. These embodiments may further be combined with embodiments wherein the insert is an immunogenic protein or a portion thereof, such as a viral protein, e.g., the coronavirus spike (S) protein or a portion thereof, or a tumor antigen. The person skilled in the art will further understand that in certain embodiments the protein comprising a binding domain is also an immunogenic protein or a portion thereof and vice versa. For example, the receptor binding domain of a viral entry protein, such as the receptor binding domain of the coronavirus spike (S) protein is a protein comprising a binding domain as well as an immunogenic protein or a portion thereof. In case two or more viral proteins (VPs) comprising different inserts are used, different insertion sites in each viral protein may be used.

In one embodiment, the top of VR-VIII corresponds to about amino acids 585 to 592 (I-585 to I-592) of VP1 AAV1 having the amino acid sequence of SEQ ID NO: 1, VP1 AAV2 having the amino acid sequence of SEQ ID NO: 2, VP1 AAV3 having the amino acid sequence of SEQ ID NO: 3, VP1 AAV6 having the amino acid sequence of SEQ ID NO: 6, VP1 AAV7 having the amino acid sequence of SEQ ID NO: 7, VP1 AAV8 having the amino acid sequence of SEQ ID NO: 8, VP1 AAV9 having the amino acid sequence of SEQ ID NO: 9 or VP1 AAV10 having the amino acid sequence of SEQ ID NO: 10, or to about amino acids 583 to 589 (I-583 to I-589) of VP1 AAV4 having the amino acid sequence of SEQ ID NO: 4, or to about amino acids 574 to 580 (I-574 to I-580) of VP1 AAVS having the amino acid sequence of SEQ ID NO: 5. Alternatively the top of VR-VIII may be defined as the 8 amino acids downstream of the conserved glutamine corresponding to Q584 of VP1 AAV2 having the amino acid sequence of SEQ ID NO: 2.

The top of VR-IV corresponds to about amino acids 450 to 460 (I-450 to I-460) of VP1 AAV1 having the amino acid sequence of SEQ ID NO: 1, VP1 AAV2 having the amino acid sequence of SEQ ID NO: 2, VP1 AAV3 having the amino acid sequence of SEQ ID NO: 3, VP1 AAV6 having the amino acid sequence of SEQ ID NO: 6, VP1 AAV7 having the amino acid sequence of SEQ ID NO: 7, VP1 AAV8 having the amino acid sequence of SEQ ID NO: 8, VP1 AAV9 having the amino acid sequence of SEQ ID NO: 9 or VP1 AAV10 having the amino acid sequence of SEQ ID NO: 10, or to about amino acids 445 to 455 (I-445 to I-455) of VP1 AAV4 having the amino acid sequence of SEQ ID NO: 4, or to about amino acids 439 to 449 (I-439 to I-449) of VP1 AA5 having the amino acid sequence of SEQ ID NO: 5. Alternatively the top of VR-IV may be defined as from 12 amino acids to 5 amino acids upstream of the conserved phenylalanine corresponding to F462 of VP1 AAV2 having the amino acid sequence of SEQ ID NO: 2.

Suitable insertions sites at the top of VR-VIII and/or VR-IV for AAV1 to AAV10 are further disclosed in Tables 1 (VR-VIII) and Table 2 (VR-IV) below.

TABLE 1
Exemplary insertion sites in VR-VIII provided as amino
acid position of VP1 of the respective AAV type.

| | | Exemplary | Exemplary |
|---|---|---|---|
| | Top of | insertion | specific |
| Corresponding | VR-VIII | sites (between | insertion sites |
| amino acids in | (amino acid | amino acid | (amino acid |
| VP1 | position) | positions) | position) |
| AAV1 | 585 to 592 | 586 to 590, preferably | I-587 |
| (SEQ ID NO: 1) | | 587 to 589 | I-588 |
| | | | I-589 |
| AAV2 | 585 to 592 | 586 to 591, preferably | I-587 |
| (SEQ ID NO: 2) | | 587 to 589 | I-588 |
| | | | I-589 |
| AAV3B | 585 to 592 | 586 to 590, preferably | I-587 |
| (SEQ ID NO: 3) | | 587 to 589 | I-588 |
| | | | I-589 |
| AAV4 | 583 to 589 | 584 to 588, preferably | I-585 |
| (SEQ ID NO: 4) | | 585 to 587 | I-586 |
| | | | I-587 |
| AAV5 | 574 to 580 | 575 to 579, preferably | I-576 |
| (SEQ ID NO: 5) | | 576 to 578 | I-577 |
| | | | I-578 |
| AAV6 | 585 to 592 | 586 to 590, preferably | I-587 |
| (SEQ ID NO: 6) | | 587 to 589 | I-588 |
| | | | I-589 |
| AAV7 | 585 to 592 | 587 to 591, preferably | I-588 |
| (SEQ ID NO: 7) | | 588 to 590 | I-589 |
| | | | I-590 |
| AAV8 | 585 to 592 | 587 to 591, preferably | I-588, |
| (SEQ ID NO: 8) | | 588 to 590 | I-589 |
| | | | I-590 |
| AAV9 | 585 to 592 | 586 to 590, preferably | I-587 |
| (SEQ ID NO: 9) | | 587 to 589 | I-588 |
| | | | I-589 |
| AAV10 | 585 to 592 | 588 to 592, preferably | I-589 |
| (SEQ ID NO: 10) | | 589 to 591 | I-590 |
| | | | I-591 |

TABLE 2
Exemplary insertion sites in VR-IV provided as amino
acid position of VP1 of the respective AAV type.

| | | Exemplary | Exemplary |
|---|---|---|---|
| | Top of | insertion | specific |
| Corresponding | VR-IV | site (between | insertion sites |
| amino acids in | (amino acid | amino acid | (amino acid |
| VP1 | position) | positions) | position) |
| AAV1 | 450 to 460 | 453 to 457, preferably | I-454, |
| (SEQ ID NO: 1) | | 454 to 456 | I-455, |
| | | | I-456 |
| AAV2 | 450 to 460 | 451 to 455, preferably | I-452 |
| (SEQ ID NO: 2) | | 452 to 454 | I-453 |
| | | | I-454 |
| AAV3B | 450 to 460 | 453 to 457, preferably | I-454 |
| (SEQ ID NO: 3) | | 454 to 456 | I-455 |
| | | | I-456 |
| AAV4 | 445 to 455 | 449 to 452, preferably | I-449 |
| (SEQ ID NO: 4) | | 449 to 451 | I-450 |
| | | | I-451 |
| AAV5 | 439 to 449 | 442 to 446, preferably | I-443 |
| (SEQ ID NO: 5) | | 443 to 445 | I-444 |
| | | | I-445 |
| AAV6 | 450 to 460 | 452 to 456, preferably | I-453 |
| (SEQ ID NO: 6) | | 453 to 455 | I-454 |
| | | | I-455 |
| AAV7 | 450 to 460 | 454 to 458, preferably | I-455 |
| (SEQ ID NO: 7) | | 455 to 457 | I-456 |
| | | | I-457 |
| AAV8 | 450 to 460 | 454 to 458, preferably | I-455, |
| (SEQ ID NO: 8) | | 455 to 457 | I-456, |
| | | | I-457 |
| AAV9 | 450 to 460 | 453 to 457, preferably | I-454, |
| (SEQ ID NO: 9) | | 454 to 456 | I-455, |
| | | | I-456 |
| AAV10 | 450 to 460 | 455 to 458, preferably | I-456 |
| (SEQ ID NO: 10) | | 456 to 458 | I-457 |
| | | | I-458 |

Preferably, the AAV or AAVLP is derived from AAV serotype 1 (AAV1), 2 (AAV2), 3 (AAV3), 4 (AAV4), 5 (AAV5), 6 (AAV6), 7 (AAV7) 8 (AAV8), 9 (AAV9) or 10 (AAV10), more preferably 1 (AAV1), 2 (AAV2), 8 (AAV8) or 9 (AAV9). In one embodiment the AAV or the AAVLP is derived from AAV2 and the insertion site is between two amino acids corresponding to amino acid position 587 and 588 (AAV2 I-587) or 588 and 589 (AAV2 I-588) and/or 453 and 454 (AAV2 I-453), 454 to 455 (AAV2 I-454) or 455 to 456 (AAV2 I-455) of AAV2 VP1 having the amino acid sequence of SEQ ID NO: 2, preferably AAV2 I-587 or AAV2 I-588 or AAV2 I-453, more preferably AAV2 I-587 or AAV2 I-588. In another embodiment the AAV or AAVLP is derived from AAV1 and the insertion site is between two amino acids corresponding to amino acid position 587 and 588 (AAV1 I-587), 588 and 589 (AAV1 I-588) or 589 and 590 (AAV1 I-589) and/or 454 and 455 (AAV1 I-454), 455 and 456 (AAV1 I-455) or 456 and 457 (AAV1 I-456) of AAV1 VP1 having the amino acid sequence of SEQ ID NO: 1. In another embodiment the AAV or AAVLP is derived from AAV8 and the insertion site is between two amino acids corresponding to amino acid position 588 and 589 (AAV8 I-588) or 589 and 590 (AAV8 I-589) and/or 455 and 456 (I-455), 456 and 457 (I-456) or 457 and 458 (I-457) of AAV8 VP1 having the amino acid sequence of SEQ ID NO: 8. In yet another embodiment the AAV or the AAVLP is derived from AAV9 and the insertion site is between two amino acids corresponding to amino acid position 588 and 589 (AAV9 I-588) or 589 and 590 (AAV9 I-589) and/or 454 and 455 (I-454), 455 and 456 (I-455) or 456 and 457 (I-456) of AAV9 VP1 having the amino acid sequence of SEQ ID NO: 9.

In certain embodiments the AAV or the AAVLP comprises an insert of about 75-400 amino acids in the viral proteins, preferably an insert of about 75-350 amino acids, of about 75-300 amino acids, of about 75-260 amino acids, of about 75-250 amino acids, or of about 80-220 amino acids.

In some embodiments, the AAV or AAVP comprises an insert of about 75-390, about 75-380, about 75-370, about 75-360, about 75-350, about 75-340, about 75-330, about 75-320, about 75-310, about 75-300, about 75-290, about 75-280, about 75-270, about 75-260, about 75-250, about 75-240, about 75-230, about 75-220 amino acids, preferably of about 80-390, about 80-380, about 80-370, about 80-360, about 80-350, about 80-340, about 80-330, about 80-320, about 80-310, about 80-300, about 80-290, about 80-280, about 80-270, about 80-260, about 80-250, about 80-240, about 80-230, about 80-220 amino acids. In one embodiment the insert has about 90-390, about 90-380, about 90-370, about 90-360, about 90-350, about 90-340, about 90-330, about 90-320, about 90-310, about 90-300, about 90-290, about 90-280, about 90-270, about 90-260, about 90-250, about 90-240, about 90-230, about 90-220 amino acids, preferably about 100-390, about 100-380, about 100-370, about 100-360, about 100-350, about 100-340, about 100-330, about 100-320, about 100-310, about 100-300, about 100-290, about 100-280, about 100-270, about 100-260, about 100-250, about 100-240, about 100-230, about 100-220 amino acids. Most preferably of about 75-300 amino acids, 75-260 amino acids, about 75-250 amino acids, or about 80-220 amino acids.

The insert may be flanked by a linker comprising one or more amino acids on one or both sides, preferably selected from the group consisting of A (Ala), G (Gly), S (Ser), T (Thr), L (Leu) and combinations thereof. Each amino acid of the linker is independently selected from a group consisting of A (Ala), G (Gly), S (Ser), T (Thr), L (Leu) and combinations thereof, preferably from a group consisting of A (Ala), G (Gly), S (Ser). The person skilled in the art will understand that the linker if present comprises small amino acids. In a preferred embodiment the linker comprises A and/or G, preferably the linker consists of A and/or G. The insert may independently comprise a linker at the N-terminal side and/or the C-terminal side of the insert. Thus, in one embodiment, the linker comprises about 1 to 7 amino acids, preferably about 1-3 amino acids, more preferably about 3 amino acids at the N-terminal side and/or about 1 to 7 amino acids, preferably about 1-3 amino acids, more preferably about 2-3 amino acids at the C-terminal side of the immunogenic protein or the portion thereof or the protein comprising a binding domain. In one embodiment, the linker comprises about 1-7 amino acids on the N-terminal side and/or about 1-7 amino acids on the C-terminal side of the insert. In a further embodiment, the linker comprises about 2-3 amino acids at the N-terminal side and/or about 2-3 amino acids at the C-terminal side. In this context the term about refers to +/− one amino acid. In a specific embodiment the linker comprises 1, 2, 3, 4, 5, 6 or 7 amino acids on the N-terminal side and/or 1, 2, 3, 4, 5, 6 or 7 amino acids on the C-terminal side of the insert or any combination thereof.

In certain embodiments, the AAV or AAVLP is an AAV and comprises an ITR-flanked genome. In certain embodiment, the AAV of the present invention comprises an ITR-flanked genome and is preferably infectious. The ITR-flanked genome may comprise a transgene. For example, the ITR-flanked genome may comprise a transgene encoding an immunogenic protein or a portion thereof. Particularly in case the insert is an immunogenic protein or a portion thereof, the transgene may encode a further immunogenic protein or a portion thereof. Alternatively, the ITR-flanked genome may comprise the cap gene encoding the viral proteins (VPs) comprising an insert of about 75-400 amino acids at an insertion site (I) at the top of variable region VIII and/or variable region IV (VR-VIII and/or VR-IV) of the VPs. The genome may also comprise non-coding sequences with immune-stimulating effect, such as CpG motifs. Also, the ssDNA itself may have an immune-stimulating effect. In other embodiments the AAV or AAVLP is an AAVLP and does not comprise an ITR-flanked genome. The person skilled in the art will understand that the AAVLP is not infectious. AAV VPs forming the capsid may be VP1, VP2 and VP3, preferably at a ratio of 1:1:10. Capsids may also be formed by VP1 and VP3 only or VP3. Thus, the AAV VPs forming the capsid may also be VP1 and VP3 or may be VP3. However, VP3-only particles are non-infectious because of the absence of a PLA2 domain encoded in the VP1-unique region of the cap ORF. In all variants the AAV or AAVLP preferably has a capsid of about 60 VPs.

The AAV or AAVLP according to the invention (for the embodiments wherein the insert is an immunogenic protein or a portion thereof) is immunogenic for the inserted immunogenic protein or the portion thereof. The immunogenic protein or a portion thereof may be any immunogenic protein and any immunogenic portion thereof. In certain embodiments, the immunogenic protein or the portion thereof may be a viral, a bacterial or a parasitic protein or a portion thereof. However, the invention also encompasses embodiments, wherein the immunogenic protein is a mammalian, particularly human protein or a portion thereof. Such AAVs or AAVLPs may be used to generate antibodies against mammalian or human antigens or epitopes, particularly for targets antigens that are difficult for generating antibodies.

Suitable exemplary immunogenic proteins from viral, bacterial or parasitic origin are without being limited thereto for Tuberculosis (Mycobacterium tuberculosis), e.g. fatty acid synthase, fas; galactofuranosyltransferase, glfT2 or isoniazid inducible gene protein iniB; for Influenza (influenza virus A, B, or C including subtypes); e.g. hemagglutinin (HA), neuraminidase (NA), nucleoprotein (NP); for Dengue fever (dengue virus; DENV1-4), e.g. envelope protein E, in particular ectodomain III (EDIII) of E, for yellow fever (YFV) e.g. envelope protein, E in particular ectodomain III (EDIII) of E; for West Nile fever (WNV), e.g. envelope protein, E in particular ectodomain III (EDIII) of E; for Congenital Zika Syndrome (Zika virus (ZIKV)), e.g. envelope protein, E in particular ectodomain III (EDIII) of E; for Malaria (Plasmodium falciparum), e.g. circumsporozoite protein (CSP), erythrocyte membrane protein 1 (PfEMP1), apical membrane antigen 1 (AMA1), merozoite surface protein 1 (MSP1), merozoite surface protein 2 (MSP2), erythrocyte binding antigen-175 (EBA175)), thrombospondin-related anonymous protein (TRAP), liver-stage antigen 1 and 3 (LSA1-LSA3), PfROM1, PfROM3, PfROM4 and PfROM6; for AIDS (HIV), e.g. env gp160, nef p27, gag p55 or pol; for Pertussis (Bordetella pertussis), e.g. pertussis toxin (PT), filamentous hemagglutinin (FHA), pertactin (PRN), and fimbriae (FIM 2/3); for Pneumonia: respiratory syncytial virus (RSV), e.g. fusion (F) glycoprotein; and for Toxoplasma encephalitis (Toxoplasma gondii), e.g. Apical membrane antigen 1 (AMA1); Enolase2 (ENO2); Dense granule proteins GRA1, GRA2, GRA4, GRA6, GRA8, GRA14, GRA15, GRA10, GRA12, GRA16, and GRA24; Heat shock protein HSP70; Microneme proteins MIC1, MIC3, MIC4, MIC5, MIC13; Rhomboid proteases ROM1, ROM4, ROM5; Rhoptry proteins ROP2, ROP5, ROP16, ROP17, ROP18, ROP38; Rhoptry neck proteins RON2, RON4, RON5; Surface antigen proteins SAG1, SAG3, SAG5D.

The person skilled in the art would understand that in case of a viral protein the immunogenic protein or the portion thereof is not an AAV protein or a portion thereof. Thus, the immunogenic protein or the portion thereof may be a heterologous viral protein or a portion thereof. The term “heterologous” as used herein means that the protein or protein fragment is from a different host organism/virus.

In certain embodiments the immunogenic protein or the portion thereof is a coronavirus protein or a portion thereof. Suitable coronavirus proteins are the coronavirus spike (S) protein (such as for SARS-CoV-2 having the amino acid sequence of SEQ ID NO: 15) or a portion thereof, the coronavirus envelop protein (E protein) (such as for SARS-CoV-2 having the amino acid sequence of SEQ ID NO: 52) or a portion thereof, the membrane glycoprotein (M protein) (such as for SARS-CoV-2 having the amino acid sequence of SEQ ID NO: 53) or a portion thereof, the nucleocapsid phosphoprotein (N protein) (such as for SARS-CoV-2 having the amino acid sequence of SEQ ID NO: 51) or a portion thereof, or the ORF1ab polypeptide (replicase complex) (such as for SARS-CoV-2 having the amino acid sequence of SEQ ID NO: 68) or a portion thereof, preferably a portion of the S protein (particularly a portion having the amino acid sequence of any one of SEQ ID NOs: 11, 12, 31, 32, 33, 34, 35, 36, 37, 38, 39, 40, 41, 42, 43, 44, 45, 46, 47, 48, 49, 50 or 69), the E protein, the M protein, a portion of the N protein (particularly amino acids 179 to 419 or 212 to 411 of SEQ ID NO: 51), or a portion of the ORF1ab polyprotein (replicase complex) (particularly a portion having the amino acid sequence of any one of SEQ ID NOs: 62, 63, 64, 65, 66 or 67 or any portion of 75 to 300 amino acids of SEQ ID NO: 68, comprising at least the sequence of one of SEQ ID NOs: 54, 55, 56, 57, 58, 59, 60 or 61). Preferably the immunogenic protein is a portion of the coronavirus S protein comprising the S1 domain, the S2 domain or the receptor binding domain, preferably comprising the coronavirus S protein receptor binding domain (RBD). More preferably the immunogenic protein is from SARS-CoV-2 and the immunogenic protein is a portion of the SARS-CoV-2 protein, such as comprising the RBD (amino acids 319 to 529 of SEQ ID NO: 15) or a portion thereof. The RBD comprises a core and a receptor-binding motive (RBM; amino acids 437 to 507 of SEQ ID NO: 15) (Shang et al, Nature, 2020, 581(7807): 221-224 and supplements). Additional T cell epitopes have been identified between amino acids 300 and 333, thus the immunogenic portion of the SARS-CoV-2 protein may have amino acids 300-507 of SEQ ID NO: 15 (SEQ ID NO: 38) or amino acids 300-505 of SEQ ID NO: 15 (SEQ ID NO: 69). Thus, in some embodiments the immunogenic protein or a portion thereof is a portion of the SARS-CoV-2 protein comprising the RBM, preferably comprising an amino acid sequence of SEQ ID NO: 11, 12, 36, 37, 38 or 69. In one embodiment the portion of the SARS-CoV S protein comprises an amino acid sequence of SEQ ID NOs: 11, 12, 31, 32, 33, 34, 35, 36, 37, 38, 39, 40, 41, 42, 43, 44, 45, 46, 47, 48, 49, 50 or 69, preferably of SEQ ID NOs: 11, 12, 34, 35, 36, 37, 38, 42 or 69 or an amino acid sequence having at least about 90% identity with the amino acid sequence of SEQ ID NOs: 11, 12, 31, 32, 33, 34, 35, 36, 37, 38, 39, 40, 41, 42, 43, 44, 45, 46, 47, 48, 49, 50 or 69 preferably of SEQ ID NOs: 11, 12, 34, 35, 36, 37, 38, 42 or 69. In one embodiment the immunogenic portion of the SARS-CoV S protein comprises an amino acid sequence having at least about 95%, at least about 98%, at least about 99% and more preferably 100% sequence identity with the amino acid sequence of SEQ ID NOs: 11, 12, 31, 32, 33, 34, 35, 36, 37, 38, 39, 40, 41, 42, 43, 44, 45, 46, 47, 48, 49, 50 or 69, preferably 11, 12, 34, 35, 36, 37, 38, 42 or 69. For example, a protein with the amino acid sequence of SEQ ID NO: 69 comprises amino acids 1-206 of SEQ ID NO: 38 and hence about 99% sequence identity with SEQ ID NO: 38. In one embodiment the immunogenic portion of the SARS-CoV S protein comprises an amino acid sequence of at least 75 amino acids of SEQ ID NOs: 11, 12, 31, 32, 33, 34, 35, 36, 37, 38, 39, 40, 41, 42, 43, 44, 45, 46, 47, 48, 49, 50 or 69, preferably 11, 12, 34, 35, 36, 37, 38, 42 or 69, or an amino acid sequence having at least about 90% identity with at least 75 amino acids of the amino acid sequence of SEQ ID NOs: 11, 12, 31, 32, 33, 34, 35, 36, 37, 38, 39, 40, 41, 42, 43, 44, 45, 46, 47, 48, 49, 50 or 69, preferably 11, 12, 34, 35, 36, 37, 38, 42 or 69. In one embodiment the immunogenic portion of the SARS-CoV S protein comprises at least about 95%, at least about 98%, at least about 99% and preferably 100% of at least 75, at least 80, at least 100, at least 125, at least 150, at least 190 or at least 195 of the amino acid sequence of SEQ ID NO: 11, 12, 31, 32, 33, 34, 35, 36, 37, 38, 39, 40, 41, 42, 43, 44, 45, 46, 47, 48, 49, 50 or 69, preferably 11, 12, 34, 35, 36, 37, 38, 42 or 69. In one embodiment the immunogenic portion of the SARS-CoV S protein comprises an amino acid sequence having at least about 95%, at least about 98%, at least about 99% and preferably 100% of at least 200, at least 225 or at least 253 amino acids of the amino acid sequence of SEQ ID NO: 12, an amino acid sequence having at least about 95%, at least about 98%, at least about 99% and preferably 100% sequence identity with at least 175, at least 190 or at least 196 amino acids of the amino acid sequence of SEQ ID NO: 11, or an amino acid sequence having at least about 95%, at least about 98%, at least about 99% and preferably 100% with at least 175, at least 200 or at least 205 amino acids of the amino acid sequence of SEQ ID NO: 69.

In certain embodiments the AAV or AAVLP according to the invention comprises an insert of about 75-400 amino acids (preferably 75-300 amino acids) in the viral proteins (VPs) forming the capsid at an insertion site (I) at the top of variable region VIII (VR-VIII) and at the top of variable region IV (VR-IV), wherein the insert is an immunogenic protein or a portion thereof and wherein the immunogenic protein or a portion thereof inserted at the top of VR-VIII and the immunogenic protein or a portion thereof inserted at the top of VR-IV may be the same or different, wherein different means a different immunogenic protein or an immunogenic portion from a different protein or a different immunogenic portion from the same protein. The immunogenic protein or the immunogenic portions from different proteins are in case of a pathogen preferably from the same pathogen (such as the same bacteria, the same virus or the same parasite). In certain embodiments, the immunogenic protein or a portion thereof inserted at the top of VR-VIII and the immunogenic protein or a portion thereof inserted at the top of VR-IV are different (such as an immunogenic protein or an immunogenic portion from a different protein or a different immunogenic portion from the same protein). Thus, the AAV or AAVLP may comprise a first insert at an insertion site at the top of VR-VIII, wherein the first insert is a first immunogenic protein or a portion thereof and a second insert at an insertion site at the top of VR-IV, wherein the second (or further) insert is a second immunogenic protein or a portion thereof. In other or additional embodiments, the AAV or AAVLP according to the invention may also be formed by 2 or more (preferably 2) viral proteins comprising different inserts of a least about 75-400 amino acids preferably at least about 75-300 amino acids, wherein the different inserts are each an immunogenic protein or a portion thereof, either an immunogenic protein or an immunogenic portion from a different protein or a different immunogenic portion from the same protein. Thus, the AAV or AAVLP formed by 2 or more (preferably 2) viral proteins comprising a first and a second (or further) insert of a least about 75-300 amino acids at the same or a different insertion site, wherein the first insert is a first immunogenic protein or a portion thereof and the second (or further) insert is a second immunogenic protein or a portion thereof. In yet another embodiment, the AAV comprises an ITR-flanked genome comprising a transgene encoding a further immunogenic protein or a portion thereof. The immunogenic protein or the portion thereof may be the same or different from the immunogenic protein or the portion thereof inserted at an insertion site at the top of VR-VIII and/or VR-IV. Also, in this context different means a different immunogenic protein or an immunogenic portion from a different protein or a different immunogenic portion from the same protein. These embodiments may be combined, thus, the AAV may comprise a genome encoding an immunogenic protein or a portion thereof and/or different immunogenic protein or a portion thereof inserted at the top of VR-VIII and VR-IV and/or may be formed by 2 or more viral proteins comprising different inserts. These embodiments may further be combined with embodiments wherein the insert is a protein comprising a binding domain, such as an antibody or an antibody fragment.

The immunogenic proteins or the portion thereof for all the above embodiments may be a coronavirus protein or a portion thereof. Suitable coronavirus proteins are the coronavirus spike (S) protein (such as for SARS-CoV-2 having the amino acid sequence of SEQ ID NO: 15) or a portion thereof the coronavirus envelop protein (E protein) (such as for SARS-CoV-2 having the amino acid sequence of SEQ ID NO: 52) or a portion thereof, the membrane glycoprotein (M protein) (such as for SARS-CoV-2 having the amino acid sequence of SEQ ID NO: 53) or a portion thereof the nucleocapsid phosphoprotein (N protein) (such as for SARS-CoV-2 having the amino acid sequence of SEQ ID NO: 51) or a portion thereof, or the ORF1ab polypeptide (replicase complex) (such as for SARS-CoV-2 having the amino acid sequence of SEQ ID NO: 68) or a portion thereof. In certain embodiments, the one or more (or the first and the further) immunogenic protein or a portion thereof may be selected from a portion of the S protein (particularly a portion having the amino acid sequence of any one of SEQ ID NOs: 11, 12, 31, 32, 33, 34, 35, 36, 37, 38, 39, 40, 41, 42, 43, 44, 45, 46, 47, 48, 49, 50 or 69, preferably SEQ ID NOs: 11, 12, 34, 35, 36, 37, 38, 42 or 69), the E protein, the M protein, a portion of the N protein (particularly amino acids 179 to 419 or 212 to 411 of SEQ ID NO: 51), a portion of the ORF1ab polyprotein (replicase complex) (particularly a portion having the amino acid sequence of any one of SEQ ID NOs: 62, 63, 64, 65, 66 or 67 or any portion of 75 to 300 amino acids of SEQ ID NO: 68, comprising at least the sequence of one of SEQ ID NOs: 54, 55, 56, 57, 58, 59, 60 or 61), an any combination thereof. If the AAV or AAVLP comprises more than one immunogenic protein or a portion thereof (inserted into the capsid or inserted into the capsid and encoded by the genome) the first and the further immunogenic proteins or portions thereof are preferably different, wherein different means a different immunogenic protein or an immunogenic portion from a different protein or a different immunogenic portion from the same protein.

Preferably the immunogenic protein is a portion of the coronavirus S protein comprising the S1 domain, the S2 domain or the receptor binding domain, preferably comprising the coronavirus S protein receptor binding domain (RBD) and/or the receptor binding motif (RBM). More preferably the immunogenic protein is from SARS-CoV-2 and the immunogenic protein is a portion of the SARS-CoV-2 protein comprising the RBD and/or the RBM. In one embodiment the portion of the SARS-CoV S protein comprises an amino acid sequence of SEQ ID NOs: 11, 12, 31, 32, 33, 34, 35, 36, 37, 38, 39, 40, 41, 42, 43, 44, 45, 46, 47, 48, 49, 50, 69, or a combination thereof, preferably 11, 12, 34, 35, 36, 37, 38, 42 or 69 or an amino acid sequence having at least about 90% identity with the amino acid sequence of SEQ ID NOs: 11, 12, 31, 32, 33, 34, 35, 36, 37, 38, 39, 40, 41, 42, 43, 44, 45, 46, 47, 48, 49, 50, 69 or a combination thereof, preferably 11, 12, 34, 35, 36, 37, 38, 42 or 69.

Various S protein sequences of SARS-CoV-2 are available at GenBank, such as GenBank accession numbers (protein-id): MN_908947 (QHD434616.1), MN_988668 (QHQ62107.1), NC_045512 (YP_009724390.1), MN_938384.1 (QHN73795.1), MN_975262.1 (QHN73810.1), MN_985325.1 (QHQ60594.1), MN_988713.1 (QHQ62877.1), MN_994467.1 (QHQ71963.1), MN_994468.1 (QHQ71973.1), and MN_997409.1 (QHQ82464.1), which show 100% sequence identity. However, minor variations have previously been reported in the SARS-CoV-2 S protein. For example, the following substitutions have been described by Wrapp et al. (Science, 2020, 367: 1260-1263) in clinical isolates F32I, H49Y, S247R, N354D, D364Y, V367F, D614G, V1129L and E1262G. Moreover, the substitutions H49Y and V860Q have been reported by Wang et al. (J. Med. Virol. Mar. 13, 2020: 1-8). Further homology analysis of the published SARS-CoV-2 sequences by the same authors revealed a nucleotide homology of the S protein of 99.82% to 100% and an amino acid homology of the S protein of 99.53% to 100%. However, further substitutions have been and will be identified over time, such as N349K and E484K. SARS-CoV-2 lineages and particularly variants of concern continue to develop and are continuously monitored and sequenced and hence the person skilled in the art knows how to access the most recent designated or assigned sequences for a respective lineage or variant.

In the context of the present invention, the term “at least 90% sequence identity with” refers to a protein that has at least 90% of a specific amino acid sequence and hence may differ in the amino acid sequence and/or the nucleic acid sequence encoding the amino acid sequence of the reference sequence, such as the amino acid sequence of SEQ ID NO: 11, 12 or 69, by less than 10% and the sequence identity can be easily determined by sequence alignment. The variant protein or the portion thereof, such as from the S protein, may be of natural origin, e.g. a mutant version or a variation of the portion of the S protein of SARS-CoV-2 having the amino acid sequence of SEQ ID NO: 11, 12 or 69, or an engineered protein, e.g. an engineered glycoprotein derivative, which has been modified by introducing site directed mutations or cloning, or a combination thereof. It is known that the usage of codons is different between species. Thus, when expressing a heterologous protein in a target cell, it may be necessary, or at least helpful, to adapt the nucleic acid sequence to the codon usage of the target cell. Methods for designing and constructing derivatives of a given protein are well known to the person skill in the art. Adapting the nucleic acid sequence to the codon usage of the target cell is also known as codon-optimization.

The immunogenic protein or a portion thereof may also be another SARS-CoV-2 protein or a portion thereof, preferably a SARS-CoV-2 N protein or a portion thereof. In preferred embodiments the SARS-CoV-2 N protein or a portion thereof comprises 75-400 or 75-300 amino acids of the sequence of SEQ ID NO: 51 or a sequence having at least 95% sequence identity with 75-400 or 75-300 amino acids of SEQ ID NO: 51. Preferably the portion of the SARS-CoV-2 N protein comprises amino acid amino acids 179 to 419 or 212 to 411 of SEQ ID NO: 51 or a sequence having at least 90%, at least 95%, at least 97%, at least 98%, or at least 99% sequence identity with amino acids amino acids 179 to 419 or 212 to 411 of the sequence of SEQ ID NO: 51. In one embodiment, the portion of the SARS-CoV-2 N protein has an amino acid sequence having at least 98% to 100% sequence identity with amino acids 179 to 419 or 212 to 411 of the sequence of SEQ ID NO: 51. In certain embodiments, the AAV or AAVLP according to the invention is an AAV and the immunogenic protein or the portion thereof is a corona virus protein or a portion thereof such as the corona virus spike (S) protein or a portion thereof or alternatively the corona virus E protein, M protein or the N protein or a portion thereof and the AAV further comprises an ITR-flanked genome comprising a transgene encoding a further immunogenic protein or a portion thereof, wherein the further immunogenic protein or a portion thereof is selected from the group consisting of a portion of corona virus S protein E protein, M protein or N protein. In certain embodiments the further immunogenic protein or a portion thereof encoded by the ITR-flanked genome is different from the immunogenic protein or a portion thereof inserted in the VPs of the AAV according to the invention, wherein different means a different protein or a different portion of the same protein. In one embodiment the immunogenic protein is a portion of the coronavirus S protein comprising the receptor binding domain selected from a portion of the S protein (particularly a portion comprising the amino acid sequence of any one of SEQ ID NOs: 11, 12, 31, 32, 33, 34, 35, 36, 37, 38, 39, 40, 41, 42, 43, 44, 45, 46, 47, 48, 49, 50 or 69), the E protein, the M protein, a portion of the N protein (particularly comprising amino acids 179 to 419 or 212 to 411 of SEQ ID NO: 51), a portion of the ORF1ab polyprotein (replicase complex) (particularly a portion having the amino acid sequence of any one of SEQ ID NOs: 62, 63, 64, 65, 66 or 67 or any portion of 75 to 300 amino acids of SEQ ID NO: 68, comprising at least the sequence of one of SEQ ID NOs: 54, 55, 56, 57, 58, 59, 60 or 61). In certain embodiments the AAV or AAVLP according to the invention is an AAV and the immunogenic protein or the portion thereof is a portion of the corona virus spike (S) protein comprising an amino acid sequence selected from the group consisting of a portion having the amino acid sequence of SEQ ID NOs: 11, 12, 31, 32, 33, 34, 35, 36, 37, 38, 39, 40, 41, 42, 43, 44, 45, 46, 47, 48, 49 and 50 and the AAV further comprises an ITR-flanked genome comprising a transgene encoding a further immunogenic protein or a portion thereof, wherein the further immunogenic protein or a portion thereof comprises the amino acid sequence of the group consisting of SEQ ID NOs: 11, 12, 31, 32, 33, 34, 35, 36, 37, 38, 39, 40, 41, 42, 43, 44, 45, 46, 47, 48, 49, 50, 52, 53, 62, 63, 64, 65, 66, 67, and amino acids 179 to 419 or 212 to 411 of SEQ ID NO: 51. The N protein (particularly a portion of the N protein comprising amino acids 179 to 419 or 212 to 411 of SEQ ID NO: 51) is considered to mainly elicit a T cell response and may therefore be particularly suitable for host cell expression of the genome encoded transgene. Without being bound by theory, it is expected that the surface exposed immunogenic protein or a portion thereof inserted into the VPs mainly elicits a humoral immune response.

For a vaccine against a pathogen, it may be beneficial if the vaccine targets multiple immunogenic proteins or a portion thereof, preferably a further structural proteins, such as the N protein, as this reduces the risk of immune-evasion due to mutations, e.g., in the S proteins.

In other embodiments, the immunogenic protein or the portion thereof is a tumor antigen. Exemplary suitable tumor antigens are, without being limited thereto carcinoembryonic antigen (CEA), epithelial growth factor receptor (EGFR), folate binding protein (FBP), GD2, GD3, human epidermal growth factor receptor 2 (HER2, erb-B2), melanoma antigen A1 (MAGE-A1), mesothelin (MSLN), prostate stem cell antigen (PSCA), prostate-specific membrane antigen (PSMA), mucin-1 (MUC1), glypican-3 (GPC3), Wilm's tumor protein (WT1), epithelial cell adhesion molecule (EpCAM), B-cell maturation antigen (BCMA), tyrosine-protein kinase transmembrane receptor (ROR1), or minor or major histocompatibility complex-associated tumor-specific (TSA) and tumor-associated antigens (TAA) (such as BCR-ABL fusion, Melanoma-associated antigen 3 (MAGE-A3), Glycoprotein 100 (gp100), Cancer/testis antigen 1 (LAGE2 or NY-ESO-1), Epstein—Barr virus latent membrane protein 1 (LMP1), P2X purinoceptor 7 (P2RX7), Diphthamide biosynthesis protein 1 (DPH1)).

The AAV or AAVLP according to the invention is a platform technology that can also be exploited by inserting targeting molecules (e.g., an antibody-derived protein or an antibody mimetic) that bind as one binding unit of a binding pair to the other binding unit of the binding pair (e.g., an antigen). The AAV or AAVLP according to the invention (for the embodiments wherein the insert is a protein comprising a binding domain) has a cell tropism conferred by the protein comprising a binding domain. The protein comprising a binding domain is one binding unit of a binding pair (such as a protein comprising an antigen-binding domain specific for a target antigen). Thus, the insert is a protein comprising a binding domain specific for a binding target and the protein comprising a binding domain, i.e., the one binding unit of the binding pair, determines the tropism of the AAV or AAVLP for a target cell expressing the binding target on its surface (e.g., ligand or receptor or antigen), i.e., the other binding unit of the binding pair (such as a target antigen binding to a protein comprising an antigen-binding domain). Suitable binding pairs, without being limited thereto are antibody-derived proteins comprising an antigen binding portion (such as nanobodies or single-chain antibodies) and antigens, preferably a single-domain antibody (sdAb) or a single chain variable fragment (scFv), or antibody mimetics (such as an anticalin, an affibody, an adnectin, a monobody, a DARPin, an affimer, or an affitin). Suitable binding pairs, without being limited thereto are a protein comprising an antigen-binding domain and its antigen (e.g., a single-domain antibody (sdAb), a single chain variable fragment (scFv) or an antibody mimetic and its antigen), a protein comprising a receptor-binding domain and a receptor (e.g., the coronavirus spike (S) protein and ACE-2 receptor; an antibody Fc-region (e.g., scFc) and an Fc receptor), and a ligand-binding domain and a ligand (e.g., PD-1 and PD-L1). Thus, in certain embodiments, the AAV tropism is determined by the insert of about 75-400, preferably 75-300 amino acids in the viral proteins, wherein the insert is a protein comprising a binding domain, such as a protein comprising an antigen-binding domain.

Suitable target antigens, without being limited thereto are tumor antigens for targeting cancer cells (e.g., carcinoembryonic antigen (CEA), epithelial growth factor receptor (EGFR), folate binding protein (FBP), GD2, GD3, human epidermal growth factor receptor 2 (HER2, erb-B2), melanoma antigen A1 (MAGE-A1), mesothelin (MSLN), prostate stem cell antigen (PSCA), prostate-specific membrane antigen (PSMA), mucin-1 (MUC1), glypican-3 (GPC3), Wilm's tumor protein (WT1), epithelial cell adhesion molecule (EpCAM), B-cell maturation antigen (BCMA), tyrosine-protein kinase transmembrane receptor (ROR1), or minor or major histocompatibility complex-associated tumor-specific (TSA) and tumor-associated antigens (TAA) (such as BCR-ABL fusion, Melanoma-associated antigen 3 (MAGE-A3), Glycoprotein 100 (gp100), Cancer/testis antigen 1 (LAGE2 or NY-ESO-1), Epstein-Barr virus latent membrane protein 1 (LMP1), P2X purinoceptor 7 (P2RX7), Diphthamide biosynthesis protein 1 (DPH1)) and/or cell type specific antigens for retargeting the AAV or AAVLP to cells expressing said antigen, particularly a surface antigen, such as a surface receptor, such as to cells not susceptible for the wild-type AAV serotype, e.g., endothelial cells or specific neuronal cell types. Examples of antigen targets that mediate cell specificity are, without being limited thereto CD4, CD8, CD11b, CD16, CD19, CD133 (prominin), CD105 (endoglin), CD146 (melanoma cell adhesion molecule), CD30, CD32, CD33, CD34, CD36, CD40, CD64, CD68, CD80, CD86, CD163, CD206, CD209, CD301, excitatory amino acid transporter 1, (SLC1A3), excitatory amino acid transporter 2 (SLC1A2), neural/glial antigen 2 (NG2), EGF-like module-containing mucin-like hormone receptor-like 1 (EMR1), folate receptor 1 (FOLR1), dopamine active transporter (DAT or SLC6A3), platelet-derived growth factor receptor (PDGFR), vesicular acetylcholine transporter (VAChT), vesicular inhibitory amino acid transporter (SLC32A1), vesicular glutamate transporters 1 and 2 (SLC17A7 and SLC17A6), or serotonin transporter (SLC6A4).

The term “protein comprising an antigen-binding domain” refers to a protein binding to a specific antigen, including antibody-derived proteins and antibody mimetics, comprising an antigen-binding site capable of binding selectively to a target antigen. Antibody mimetics are proteins that bind to specific antigens in a manner similar to antibodies, but that are not structurally related to antibodies. Proteins of about 75 to 400 amino acids, preferably 75-300 amino acids comprising an antigen-binding domain may be any format including without being limited thereto an sdAb, a single chain variable fragment (scFv), an anticalin, an affibody, an adnectin, a monobody, a DARPin, an affimer, or an affitin, preferably an antibody-derived protein comprising an antigen binding domain selected from the group consisting of an sdAb and a single chain variable fragment (scFv) or an antibody mimetic comprising an antigen-binding domain selected from the group consisting of an anticalin, an affibody, an adnectin, a monobody, a DARPin, an affimer, and an affitin. A single-domain antibody (sdAb) may also be referred to as nanobody. The person skilled in the art will understand that the protein may comprise more than one antigen-binding domain and hence may be multivalent, preferably bivalent (e.g., a bivalent sdAb or a bivalent anticalin or any other bivalent antibody mimetic). Moreover, the protein may be multispecific, preferably bispecific, i.e., having a specificity for two different antigens (e.g., a bispecific sdAb or a bispecific anticalin or any other bivalent antibody mimetic).

In certain embodiments the insert is a protein comprising a binding domain, such as an antigen-binding domain and the AAV or AAVLP is preferably an AAV comprising an ITR-flanked genome and is infectious. More preferably, the ITR-flanked genome comprises a transgene. The AAV according to the invention, wherein the insert is a protein comprising a binding domain, and wherein the AAV comprises an ITR-flanked genome comprising a transgene and is infectious is particularly useful for use in gene therapy. In gene therapy a gene is delivered into specific cell types and its expression leads to a therapeutic effect. Exemplary gene therapies include, without being limited thereto, gene augmentation, gene supplementation, gene addition or gene editing (including CRISPR-Cas or other technologies). Generally, the retargeted AAVs according to the invention are suitable for ex vivo, in vivo, and in situ gene therapy. In ex vivo gene therapy (also referred to in vitro gene therapy), the target cells are removed from the patient's body, engineered either by the addition of the therapeutic gene or by other genetic manipulations that allow correction of the phenotype of the disease and the engineered cells are subsequently re-infused to the patient. It is particularly applicable to blood diseases (including chimeric antigen receptor (CAR) based technologies, such as CAR T-cells and CAR NK-cells). In in vivo gene therapy, the retargeted AAV according to the invention is administered systemically in the blood circulation or the cerebrospinal fluid of the patient, and depending on the disease targets specific cells, such as in the brain, the spinal canal, or the liver. In in situ gene therapy retargeted AAV according to the invention is administered in situ, i.e., to a specific organ or area in the body of the patient either through direct injection, e.g., into the tumor (e.g., in the case of melanoma) or into suitable brain areas (e.g., in the case of neuropathies) or by an insertion of a catheter, e.g., in the case that the organ to be treated is the heart. Preferably the gene therapy is in vivo or in situ gene therapy. In certain embodiments, the protein comprising a binding domain is a protein comprising an antigen-biding domain specific for a tumor antigen and the ITR-flanked genome comprises a suicide gene, preferably for use in treating cancer.

The person skilled in the art will understand that the coronavirus spike (S) protein or a portion thereof is also a binding protein comprising a binding domain. The S protein binds to the cellular receptor ACE-2. Thus, the insert may also be an immunogenic protein or a portion thereof and a protein comprising a binding domain.

In a further aspect the invention relates to a pharmaceutical composition comprising the AAV or AAVLP according to the invention, and preferably further at least one pharmaceutically acceptable excipient. In the context of the present invention, the term “excipient” refers to a natural or synthetic substance formulated alongside the active ingredient of a medication. Suitable excipients include antiadherents, binders, coatings, disintegrants, flavors, colors, lubricants, glidants, sorbents, preservatives and sweeteners. An excipient may also include an adjuvant. In certain embodiments the pharmaceutical composition comprising the AAV or AAVLP according to the invention further comprises at least one adjuvant and at least one further pharmaceutically acceptable excipient. The pharmaceutical composition according to the invention may comprise two or more AAV or AAVLP according to the invention or one or more AAV and one or more AAVLP according to the invention (referred to as two or more AAV and/or AAVLP in the following), wherein the two or more AAV and/or AAVLP may be present in a fixed dosage form (i.e., physically mixed) and/or may be provided in separate dosage forms. Wherein the two or more AAV and/or AAVLP each comprise a different immunogenic protein or a portion thereof and/or protein comprising a binding domain inserted at the top of VR-VIII and/or VR-IV, wherein different may be an immunogenic protein, or an immunogenic portion from a different protein or a different immunogenic portion from the same protein, or an immunogenic protein or a portion thereof and a protein comprising a binding domain, or two different proteins comprising a binding domain (i.e., with a specificity for two different targets, such as antigens, ligands and/or receptors). Preferably, the immunogenic protein or the immunogenic portions from different proteins are in case of a pathogen from the same pathogen (such as the same bacteria, the same virus or the same parasite).

In the context of the present invention, the term “pharmaceutically acceptable” refers to molecular entities and other ingredients of pharmaceutical compositions that are physiologically tolerable and do not typically produce unwanted reactions when administered to a mammal (e.g., human). The term “pharmaceutically acceptable” may also mean approved by a regulatory agency of a Federal or a state government or listed in the U.S. Pharmacopeia or other generally recognized pharmacopeia for use in mammals, and, more particularly, in humans.

In the context of the present invention the AAV or AAVLP or the pharmaceutical formulation comprising said AAV or AAVLP may be adapted to be administered via the intranasal mucosal, sublingual, oral, buccal, intravenous, intramuscular, intraperitoneal or subcutaneous route, preferably the intranasal mucosal, sublingual, intravenous, or subcutaneous route. In one embodiment, the AAV or AAVLP or the pharmaceutical formulation comprising said AAV or AAVLP may be adapted to be administered by inhalation via the intranasal, oral and/or mucosal route.

### Therapeutic Uses of the AAV Or AAVLP According to the Invention

In further aspect the AAV or AAVLP or the pharmaceutical composition according to the invention is for use in therapy. In one embodiment the AAV or the AAVLP or the pharmaceutical composition according to the invention is for use as a vaccine, preferably in humans. In this context the insert is an immunogenic protein or a portion thereof as described herein. The term “vaccine” as used herein refers to an agent which is able to induce an immune response in a subject upon administration. A vaccine can preferably prevent, ameliorate or treat a disease. In the context of the present invention the vaccine may be a protective or prophylactic vaccine for preventing, e.g., an infection with a pathogen, or the vaccine may be a therapeutic vaccine for treating, e.g., cancer. The person skilled in the art will however understand that a vaccine may also be therapeutic in the case of a viral, bacterial or parasitic infection, e.g., ameliorating the disease or symptoms following onset of disease. The vaccine comprising the AAV or AAVLP of the present invention is a subunit vaccine using the AAV or AAVLP as a carrier. The carrier may be inert or serve as an adjuvant by providing immune stimuli such as ssDNA or antigenic and immunogenic epitopes.

In a specific aspect, the invention relates to the AAV or AAVLP or the pharmaceutical composition according to the invention for use in the treatment or the prevention of a disease induced by a virus, a bacterium or a parasite, wherein the immunogenic protein or the portion thereof is an immunogenic protein of said virus, bacterium or parasite as specified above for the AAV or the AAVLP according to the invention. In one embodiment the disease is a coronavirus respiratory syndrome and the immunogenic protein or a portion thereof is the portion of a coronavirus spike (S) protein. Preferably, the disease is coronavirus disease 2019 (COVID-19) and the immunogenic protein or the portion thereof is a portion of the SARS-CoV-2 spike (S) protein. In a specific aspect, the immunogenic protein or the portion thereof in the AAV or AAVLP according to the invention comprises a portion of the SARS-CoV-2 spike (S) protein and the AAV or AAVLP is for use in inducing an immune response against SARS-CoV-2. In certain embodiments of the AAV or AAVLP for use according to the invention, the portion of the SARS-CoV-2 spike (S) protein comprises the SARS-CoV-2 S protein receptor binding domain (RBD) or a portion thereof, preferably a portion comprising the receptor binding motif (RBM). In certain embodiments the portion of the SARS-CoV-2 S protein comprises an amino acid sequence of SEQ ID NOs: 11, 12, 31, 32, 33, 34, 35, 36, 37, 38, 39, 40, 41, 42, 43, 44, 45, 46, 47, 48, 49, 50 or 69, preferably of SEQ ID NOs: 11, 12, 34, 35, 36, 37, 38, 42 or 69 or an amino acid sequence having at least 90% sequence identity with the amino acid sequence of SEQ ID NOs: 11, 12, 31, 32, 33, 34, 35, 36, 37, 38, 39, 40, 41, 42, 43, 44, 45, 46, 47, 48, 49, 50 or 69, preferably of SEQ ID NOs: 11, 12, 34, 35, 36, 37, 38, 42 or 69.

In a further specific aspect, the AAV or AAVLP or the pharmaceutical composition according to the invention is for use in the treatment or the prevention of cancer and the insert is an immunogenic protein or the portion thereof, wherein the immunogenic protein or the portion thereof is a tumor antigen or portion thereof. Alternatively or in addition, the insert may be a protein comprising a binding domain, such as an antigen-binding domain, specific for a tumor antigen as target antigen. A suitable tumor antigen as immunogenic protein and/or as target antigen, without being limited thereto, may be selected from the group consisting of carcinoembryonic antigen (CEA), epithelial growth factor receptor (EGFR), folate binding protein (FBP), GD2, GD3, human epidermal growth factor receptor 2 (HER2, erb-B2), melanoma antigen A1 (MAGE-A1), mesothelin (MSLN), prostate stem cell antigen (PSCA), prostate-specific membrane antigen (PSMA), mucin-1 (MUC1), glypican-3 (GPC3), Wilm's tumor protein (WT1), epithelial cell adhesion molecule (EpCAM), B-cell maturation antigen (BCMA) and tyrosine-protein kinase transmembrane receptor (ROR1), or minor or major histocompatibility complex-associated tumor-specific (TSA) and tumor-associated antigens (TAA) (such as BCR-ABL fusion, Melanoma-associated antigen 3 (MAGE-A3), Glycoprotein 100 (gp100), Cancer/testis antigen 1 (LAGE2 or NY-ESO-1), Epstein—Barr virus latent membrane protein 1 (LMP1), P2X purinoceptor 7 (P2RX7), Diphthamide biosynthesis protein 1 (DPH1)). The cancer to be treated may be a solid tumor or a hematological tumor. Suitable cancer to be treated may be without being limited thereto, colorectal cancer, breast cancer, hepatocellular cancer, glioma, lung cancer, particularly non-small cell lung cancer, ovarian cancer, neuroblastoma, melanoma, head and neck squamous cell carcinoma, gastric cancer, pancreatic cancer, mesothelioma, prostate cancer, hepatocellular cancer, AML or CML. The person skilled in the art will further understand that certain viral immunogenic proteins may also act as a tumor antigen, such as HCV or HPV derived antigens.

In yet a further aspect, the AAV or AAVLP or the pharmaceutical composition according to the invention is for use in therapy, particularly for use in gene therapy. In this context the insert is a protein comprising a binding domain, preferably a protein comprising an antigen-binding domain, a receptor-binding domain or a ligand-binding domain, more preferably an antigen-binding domain. Thus, the AAV or AAVLP is retargeted. For example, the antigen-binding domain may be specific for a cell type specific antigen, particularly a surface antigen, such as a surface receptor, for retargeting the AAV or AAVLP to said cell type, such as to cells not susceptible for the wild-type AAV serotype, e.g., endothelial cells or specific neuronal cell types. In a specific embodiment the antigen-binding domain may be specific for a tumor antigen, retargeting the AAV or AAVLP to tumor cell expressing said tumor antigen. More preferably the AAV or AAVLP is an AAV and the AAV comprises an ITR-flanked genome and is infectious. In certain embodiments the ITR-flanked genome comprises a transgene. In case of a tumor retargeted AAV, the transgene may be also a suicide gene.

The therapeutic use according to the invention may also comprise two or more AAV or AAVLP according to the invention to be administered or one or more AAV and one or more AAVLP according to the invention to be administered (referred to as two or more AAV and/or AAVLP to be administered in the following), wherein the two or more AAV and/or AAVLP to be administered may be present in a fixed dosage form (i.e., physically mixed) and/or may be provided in separate dosage forms. They may be administered simultaneously or at different time points. Wherein the two or more AAV and/or AAVLP each comprise a different insert (such as a different immunogenic protein or a portion thereof and/or protein comprising a binding domain) inserted at the top of VR-VIII and/or VR-IV. For an immunogenic protein or a portion thereof different may be an immunogenic protein or an immunogenic portion from a different protein or a different immunogenic portion from the same protein. Preferably, the immunogenic protein or the immunogenic portions from different proteins are in case of a pathogen from the same pathogen (such as the same bacteria, the same virus or the same parasite) or in case of cancer from the same tumor. For the prime and the boost vaccination an AAV or AAVLP according to the invention based on a different serotype carrying a same or at least overlapping immunogenic protein or a portion thereof. For a protein comprising a binding domain, different means proteins with different binding specificity.

The AAV or AAVLP for use according to the invention may be administered via the intranasal mucosal, sublingual, oral, buccal, intravenous, intramuscular, intraperitoneal or subcutaneous route, preferably the intranasal mucosal, sublingual, intravenous, or subcutaneous route. In one embodiment, the AAV or AAVLP may be administered by inhalation via the intranasal, oral and/or mucosal route.

### Methods for Producing the AAV or AAVLP According to the Invention

In yet another aspect, the invention relates to a method for producing an AAV or an AAVLP, comprising the steps of (i) preparing a cell comprising at least one DNA sequence comprising a cap gene and a rep gene, at least one DNA sequence comprising adenoviral helper sequences and optionally at least one DNA sequence comprising an ITR-flanked genome; wherein the cap gene encodes a protein comprising an insert of about 75-400, preferably about 75-300 amino acids in the viral proteins (VPs) forming the capsid at an insertion site (I) at the top of variable region VIII and/or variable region IV (VR-VIII and/or VR-IV) of the VPs, and wherein the insert is optionally flanked by a linker comprising one or more amino acids on both sides selected from the group consisting of A (Ala), G (Gly), S (Ser), T (Thr), L (Leu) and combinations thereof; (ii) cultivating the cells under conditions allowing the production of the AAV or the AAVLP; (iii) purifying the AAV or the AAVLP. In certain embodiments, the method is a method for producing a pharmaceutical composition comprising an AAV or AAVLP comprising the steps of the method of the invention and further comprising a step (iv) adding at least one pharmaceutically acceptable excipient to formulate the AAV or the AAVLP into a pharmaceutical composition. The person skilled in the art would understand that the cap and rep gene may comprise a natural AAV promoter, such as the respective wild-type AAV promoter or alternatively the natural rep and/or particularly the cap promoter may be replaced or supplemented with a different eukaryotic promoter, preferably a strong eukaryotic promoter, such as a strong mammalian promoter, e.g., CMV, RSV or SV40, to regulate and improve expression levels. The method according to the invention is an in vitro method. The term “eukaryotic promoter” and “mammalian promoter” as used herein refers to any promoter that drives gene expression in a eukaryotic or mammalian cell, including viral promoters.

In certain embodiments, the method for producing an AAV or an AAVLP, comprises the steps of (i) preparing a cell comprising at least one DNA sequence comprising a cap gene and a rep gene, at least one DNA sequence comprising adenoviral helper sequences and optionally at least one DNA sequence comprising an ITR-flanked genome; wherein the cap gene encodes a protein comprising an insert of about 75-400, preferably about 75-300 amino acids in the viral proteins (VPs) forming the capsid at an insertion site (I) at the top of variable region VIII and/or variable region IV (VR-VIII and/or VR-IV) of the VPs, wherein the insert is (a) an immunogenic protein or a portion thereof, and/or (b) a protein comprising a binding domain, and wherein the insert is optionally flanked by a linker comprising one or more amino acids on both sides selected from the group consisting of A (Ala), G (Gly), S (Ser), T (Thr), L (Leu) and combinations thereof; (ii) cultivating the cells under conditions allowing the production of the AAV or the AAVLP; (iii) purifying the AAV or the AAVLP; and optionally adding at least one pharmaceutically acceptable excipient to formulate the AAV or the AAVLP into a pharmaceutical composition. The method according to the invention is an in vitro method.

The adenoviral helper sequences are E2A, E4, and VA RNA and if not already expressed by the producer cell (e.g. HEK293 cells) E1A/E1B. Optionally a further plasmid encodes an ITR-flanked genome (such as an ITR-flanked genome comprising a transgene expression cassette).

In certain embodiments, the at least one DNA sequence comprising a cap gene and a rep gene, the at least one DNA sequence comprising adenoviral helper sequences and/or the optional at least one DNA sequence comprising an ITR-flanked genome may be independently stably expressed or transiently expressed. In specific embodiments, the method comprises (a) transfecting the mammalian cell with at least one DNA molecule comprising a cap gene and a rep gene, at least one DNA molecule comprising adenoviral helper sequences and optionally at least one further DNA molecule comprising an ITR-flanked genome, preferably wherein the DNA molecule is a plasmid or a linear DNA molecule; or (b) transducing the mammalian cell with at least one vector comprising a cap gene and a rep gene, at least one vector comprising adenoviral helper sequences and optionally at least one vector comprising an ITR-flanked genome, wherein the vector is a viral vector, preferably selected from baculovirus and Herpes simplex virus; or (c) a cell stably expressing at least one DNA sequence comprising a cap gene and a rep gene, at least one DNA sequence comprising adenoviral helper sequences and/or optionally at least one DNA sequence comprising an ITR-flanked genome, and providing the not stably expressed DNA sequences transiently by transfection and/or transduction. The at least one DNA sequence comprising a cap gene and a rep gene, the at least one DNA sequence comprising adenoviral helper sequences and/or the optional at least one DNA sequence comprising an ITR-flanked genome may be independently stably expressed or transiently expressed.

Specifically AAVs according to the invention were prepared as described below. For the production of AAVs, 15 150-mm petri dishes of HEK293T cells at 80% confluence were cotransfected with 20 μg of DNA per petri dish. The pHtW2_S1.1 or pHtW9_S1.1 AAV Rep/Cap plasmid was thereby cotransfected at equimolar ratio with an adenoviral helper plasmid (e.g. pXX6 from J. Samulski, Chapel Hill, NC; Xiao, Li and Samulski (1998) “Production of high-titer recombinant adeno-associated virus vectors in the absence of helper adenovirus.” J. Virol. 72: 2224-2232) and in case of production of full capsids with equimolar amount of a pTransgene plasmid containing an ITR-flanked CMV-eGFP cassette. After 48 h AAVs were isolated from HEK293 cell pellets which were resuspended in 150 mM NaCl, 50 mM Tris—HCl (pH 8.5), freeze—thawed several times, and treated with Benzonase (50 U/ml) for 30 min at 37° C. Cell debris was removed by centrifugation, and supernatant was further processed for iodixanol gradient. Alternatively, AAVs were isolated from cell culture supernatants after overnight precipitation at 4° C. with 8% polyethylene glycol (PEG) 8000. The PEG-AAV precipitate was then treated with Benzonase (50 U/ml) for 30 min at 37° C. and further processed for iodixanol gradient. Iodixanol gradient ultracentrifugation was performed at 70,000 rpm for 1 h and 45 min at 18° C. as described (Zolotukhin et al. (1999) “Recombinant adeno-associated virus purification using novel methods improves infectious titer and yield. Gene Ther. 6: 973-985). Virions were then harvested from the 40% iodixanol phase and titrated by DNA dot-blot hybridization with a rep probe (Girod et al. (1999) “Genetic capsid modifications allow efficient re-targeting of adeno-associated virus type 2. Nat. Med. 5: 1052-1056).

In addition to the approach described above other approaches may be utilized for the production of rAAVs. For example, other transient cotransfection methods using minicircle DNA or closed linear DNA (e.g. doggybone DNA) devoid of bacterial plasmid backbone instead of conventional plasmid DNA could be employed. Alternatively, stable mammalian producer cell lines (e.g. HeLa) transformed with plasmids encoding the rep and cap genes and optionally a transgene flanked by AAV ITRs could be employed. Such cells could be infected with wild-type Adenovirus (e.g. Ad type 5) or AAV ITR flanked genome-containing Adenovirus/AAV hybrid virus to generate the rAAV particles of choice which contain the transgene packaged into the rAAV capsid modified according to the invention. AAVLPs are basically produced according to the same method as the AAVs according to the invention, except that the mammalian cells are transformed with plasmids lacking any the ITR flanked genome or infected with wildtype Adenovirus instead of AAV ITR flanked genome-containing Adenovirus/AAV hybrid virus. Another suitable approach uses a baculovirus/Sf9 system in which two to three separate viruses (Rep baculovirus, VP baculovirus and optionally an AAV ITR flanking a transgene baculovirus) are used for infection. The resulting rAAV vectors contain the transgene packaged into the rAAV capsid modified according to the invention. AAVLPs are basically produced according to the same method as the AAVs according to the invention, except that the mammalian cells are transfected without the plasmid containing the ITR flanked genome or infected without the AAV ITR flanked genome baculovirus. Alternatively, mammalian cells (e.g. hamster BHK21 cells, HEK293 cells or derivatives) can be infected with one or two recombinant herpes simplex viruses (rHSVs) which express the AAV rep and modified cap genes and optionally a transgene flanked by AAV ITRs. Any additional helper functions are provided by the rHSV genes. The resulting rAAV vectors contain the transgene packaged into the rAAV capsid modified according to the invention. AAVLPs are basically produced according to the same method as the AAVs according to the invention, except that the mammalian cells are infected with only the rHSV which expresses the AAV rep and modified cap genes, but not with the rHSV with the transgene flanked by AAV ITRs.

The ITR-flanked genome may further comprise a transgene encoding a further immunogenic protein or a portion thereof. In certain other embodiments the AAV or AAVLP is an AAVLP not comprising a genome that is flanked by ITRs. AAV VPs forming the capsid may be VP1, VP2 and VP3, preferably at a ratio of 1:1:10. Capsids may also be formed by VP1 and VP3 only or VP3. Thus, the AAV VPs forming the capsid may also be VP1 and VP3 or may be VP3. In all variants the AAV or AAVLP preferably has a capsid of about 60 VPs. The immunogenic proteins or a portion thereof and the insertions sites may be as disclosed above for the AAV or AAVLP according to the invention. In the context of the method of the invention the immunogenic protein or the portion, the insertion sites as well as the genome may be defined as specified above for the AAV or AAVLP of the invention.

In view of the above, it will be appreciated that the invention also encompasses the following items.
1. An adeno-associated virus (AAV) or a adeno-associated virus-like particle (AAVLP) comprising an insert of about 75-400 amino acids, preferably about 75-300 amino acids, in the viral proteins (VPs) forming the capsid at an insertion site (I) at the top of variable region VIII and/or variable region IV (VR-VIII and/or VR-IV) of the VPs, wherein the insert is an immunogenic protein or a portion thereof, and wherein the insert is optionally flanked by a linker comprising one or more amino acids on one or both sides, preferably selected from the group consisting of A (Ala), G (Gly), S (Ser), T (Thr), L (Leu) and combinations thereof.
2. The AAV or AAVLP according to item 1, wherein (a) the top of VR-VIII corresponds to amino acids 585 to 592 (I-585 to I-592) of VP1 of AAV 1, 2, 3, 6, 7, 8, 9 or 10 having the amino acid sequence of SEQ ID NO: 1, 2, 3, 6, 7, 8, 9 or 10, respectively, to amino acids 583 to 589 of VP1 of AAV 4 having the amino sequence of SEQ ID NO: 4, or to amino acids 574 to 580 of VP1 of AAV 5 having the amino sequence of SEQ ID NO: 5, and/or (b) the top of VR-IV corresponds to amino acids 450 to 460 (I-450 to I-460) of VP1 of AAV 1, 2, 3, 6, 7, 8, 9 or 10 having the amino acid sequence of SEQ ID NO: 1, 2, 3, 6, 7, 8, 9 or 10, respectively, to amino acids 445 to 455 (I-445 to I-455) of VP1 of AAV 4 having the amino sequence of SEQ ID NO: 4, or to amino acids 439 to 449 (I-439 to I-449) of VP1 of AAV 5 having the amino sequence of SEQ ID NO: 5.
3. The AAV or AAVLP according to any one of the preceding items, wherein the AAV or AAVLP is derived from AAV serotype 1 (AAV1), 2 (AAV2), 8 (AAV8) or 9 (AAV9), preferably wherein
   (a) the insertion site is between two amino acids corresponding to amino acid position 587 and 588 (AAV2 I-587) or 588 and 589 (AAV2 I-588) and/or 453 and 454 (AAV2 I-453) of AAV2 VP1 having the amino acid sequence of SEQ ID NO: 2, preferably AAV2 I-587 or AAV2 I-588 or AAV2 I-453, more preferably AAV2 I-587 or AAV2 I-588;
   (b) the insertion site is between two amino acids corresponding to amino acid position 587 and 588 (AAV1 I-587), 588 and 589 (AAV1 I-588) or 589 and 590 (AAV1 I-589) and/or 454 and 455 (AAV1 I-454), 455 and 456 (AAV1 I-455) or 456 and 457 (AAV1 I-456) having the amino acid sequence of SEQ ID NO: 1;
   (c) the insertion site is between two amino acids corresponding to amino acid position 588 and 589 (AAV8 I-588) or 589 and 590 (AAV8 I-589) and/or 455 and 456 (I-455), 456 and 457 (I-456) or 457 and 458 (I-457) of AAV8 VP1 having the amino acid sequence of SEQ ID NO: 8, or
   (d) the insertion site is between two amino acids corresponding to amino acid position 588 and 589 (AAV9 I-588) or 589 and 590 (AAV9 I-589) and/or 454 and 455 (I-454), 455 and 456 (I-455) or 456 and 457 (I-456) of AAV9 VP1 having the amino acid sequence of SEQ ID NO: 9.
4. The AAV or AAVLP according to any one of the preceding items, wherein
   (a) the AAV comprises an ITR-flanked genome and is infectious, optionally wherein the ITR-flanked genome comprises a transgene encoding a further immunogenic protein or a portion thereof;
   (b) an immunogenic protein or a portion thereof is inserted at the top of VR-VIII and at the top of VR-IV and wherein the immunogenic protein or a portion thereof inserted at the top of VR-VIII and the immunogenic protein or a portion thereof inserted at the top of VR-IV are the same or different; and/or
   (c) the AAV or AAVLP is formed by 2 or more viral proteins comprising different inserts of a least about 75-400 amino acids, preferably about 75-300 amino acids, wherein the different inserts are each an immunogenic protein or a portion thereof, either an immunogenic protein or an immunogenic portion from a different protein or a different immunogenic portion from the same protein.
5. The AAV or AAVLP according to any one the preceding items, wherein the AAV or AAVLP has a capsid of about 60 VPs, wherein the VPs are
   (a) VP3;
   (b) VP1 and VP3; or
   (c) VP1, VP2 and VP3 proteins, preferably at a ratio of 1:1:10.
6. The AAV or AAVLP according to any one of the preceding items, wherein the immunogenic protein or the portion thereof is a viral, a bacterial or a parasitic protein or a portion thereof.
7 The AAV or AAVLP according to item 6, wherein the immunogenic protein or the portion thereof is
   (a) a portion of coronavirus spike (S) protein;
   (b) a portion of the SARS-CoV-2 spike (S) protein, preferably wherein the portion of the SARS-CoV-2 spike (S) protein comprises the SARS-CoV-2 S protein receptor binding domain (RBD) or a portion thereof; and/or
   (c) a portion of the SARS-CoV-2 S protein comprising an amino acid sequence of SEQ ID NO: 11, 12, 31, 32, 33, 34, 35, 36, 37, 38, 39, 40, 41, 42, 43, 44, 45, 46, 47, 48, 49, 50, or 69, preferably 11, 12, 34, 35, 36, 37, 38, 42 or 69.
8. The AAV or AAVLP according to any one of items 1 to 5, wherein the immunogenic protein or the portion thereof is a tumor antigen.
9. A pharmaceutical composition comprising the AAV or AAVLP according to of any one of items 1-8, further comprising at least one pharmaceutically acceptable excipient.
10. The AAV or AAVLP according to any one of items 1 to 8 or the pharmaceutical composition of item 9 for use as a vaccine.
11. The AAV or AAVLP according to any one of items 1 to 7 for use in the treatment or the prevention of a disease induced by a virus, a bacterium or a parasite and wherein the immunogenic protein or the portion thereof is an immunogenic protein of said virus, bacterium or parasite, respectively.
12. The AAV or AAVLP for use according to item 11, wherein the disease is a coronavirus respiratory syndrome and wherein the immunogenic protein or a portion thereof is the portion of a coronavirus spike (S) protein, preferably wherein the disease is coronavirus disease 2019 (COVI D-19) and wherein the immunogenic protein or the portion thereof is a portion of the SARS-CoV-2 spike (S) protein.
13. The AAV or AAVLP according to item 8 for use in treating or preventing cancer, wherein the immunogenic protein or the portion thereof is a tumor antigen or portion thereof.
14. The AAV or AAVLP for use according to any one of items 10-13, wherein the AAV or AAVLP is to be administered via the intranasal mucosal, sublingual, oral, buccal, intravenous, intramuscular, intraperitoneal or subcutaneous route, preferably wherein the AAV or AAVLP is to be administered by inhalation via the intranasal, oral and/or mucosal route.
15. A method for producing an AAV or an AAVLP, comprising the steps of
   (i) preparing a cell comprising at least one DNA sequence comprising a cap gene and a rep gene, at least one DNA sequence comprising adenoviral helper sequences and optionally at least one DNA sequence comprising an ITR-flanked genome;
      wherein the cap gene encodes a protein comprising an insert of about 75-400 amino acids, preferably about 75-300 amino acids, in the viral proteins (VPs) forming the capsid at an insertion site (I) at the top of variable region VIII and/or variable region IV (VR-VIII and/or VR-IV) of the VPs, wherein the insert is an immunogenic protein or a portion thereof, and wherein the insert is optionally flanked by a linker comprising one or more amino acids on both sides, preferably selected from the group consisting of A (Ala), G (Gly), S (Ser), T (Thr), L (Leu) and combinations thereof;
   (ii) cultivating the cells under conditions allowing the production of the AAV or the AAVLP;
   (iii) purifying the AAV or the AAVLP.
16. A method for producing a pharmaceutical composition comprising an AAV or an AAVLP, comprising the steps of
   (i) preparing a cell comprising at least one DNA sequence comprising a cap gene and a rep gene, at least one DNA sequence comprising adenoviral helper sequences and optionally at least one DNA sequence comprising an ITR-flanked genome;
      wherein the cap gene encodes a protein comprising an insert of about 75-400 amino acids, preferably about 75-300 amino acids, in the viral proteins (VPs) forming the capsid at an insertion site (I) at the top of variable region VIII and/or variable region IV (VR-VIII and/or VR-IV) of the VPs, wherein the insert is an immunogenic protein or a portion thereof, and wherein the insert is optionally flanked by a linker comprising one or more amino acids on both sides, preferably selected from the group consisting of A (Ala), G (Gly), S (Ser), T (Thr), L (Leu) and combinations thereof;
   (ii) cultivating the cells under conditions allowing the production of the AAV or the AAVLP;
   (iii) purifying the AAV or the AAVLP; and
   (iv) adding at least one pharmaceutically acceptable excipient to formulate the AAV or the AAVLP into a pharmaceutical composition.
17. An adeno-associated virus (AAV) or a adeno-associated virus-like particle (AAVLP) comprising an insert of about 75-400 amino acids, preferably about 75-300 amino acids, in the viral proteins (VPs) forming the capsid at an insertion site (I) at the top of variable region VIII and/or variable region IV (VR-VIII and/or VR-IV) of the VPs, and wherein the insert is optionally flanked by a linker comprising one or more amino acids on one or both sides, preferably selected from the group consisting of A (Ala), G (Gly), S (Ser), T (Thr), L (Leu) and combinations thereof.
18. The AAV or AAVLP according to item 17, wherein the insert is (a) an immunogenic protein or a portion thereof, and/or (b) a protein comprising a binding domain.
19. The AAV or AAVLP according to item 17 or 18, wherein the insert is a protein comprising a binding domain, preferably an antigen binding domain.
20. The AAV or AAVLP according to any one of items 17-19, wherein the AAV comprises an ITR-flanked genome and is infectious, preferably wherein the ITR-flanked genome comprises a transgene.
21. The AAV or AAVLP according to any one of items 17-20, wherein (a) the top of VR-VIII corresponds to amino acids 585 to 592 (I-585 to I-592) of VP1 of AAV 1, 2, 3, 6, 7, 8, 9 or 10 having the amino acid sequence of SEQ ID NO: 1, 2, 3, 6, 7, 8, 9 or 10, respectively, to amino acids 583 to 589 of VP1 of AAV 4 having the amino sequence of SEQ ID NO: 4, or to amino acids 574 to 580 of VP1 of AAV 5 having the amino sequence of SEQ ID NO: 5, and/or (b) the top of VR-IV corresponds to amino acids 450 to 460 (I-450 to I-460) of VP1 of AAV 1, 2, 3, 6, 7, 8, 9 or 10 having the amino acid sequence of SEQ ID NO: 1, 2, 3, 6, 7, 8, 9 or 10, respectively, to amino acids 445 to 455 (I-445 to I-455) of VP1 of AAV 4 having the amino sequence of SEQ ID NO: 4, or to amino acids 439 to 449 (I-439 to I-449) of VP1 of AAV 5 having the amino sequence of SEQ ID NO: 5.
22. The AAV or AAVLP according to any one of items 17-21, wherein the AAV or AAVLP is derived from AAV serotype 1 (AAV1), 2 (AAV2), 8 (AAV8) or 9 (AAV9), preferably wherein
   (a) the insertion site is between two amino acids corresponding to amino acid position 587 and 588 (AAV2 I-587) or 588 and 589 (AAV2 I-588) and/or 453 and 454 (AAV2 I-453) of AAV2 VP1 having the amino acid sequence of SEQ ID NO: 2, preferably AAV2 I-587 or AAV2 I-588 or AAV2 I-453, more preferably AAV2 I-587 or AAV2 I-588;
   (b) the insertion site is between two amino acids corresponding to amino acid position 587 and 588 (AAV1 I-587), 588 and 589 (AAV1 I-588) or 589 and 590 (AAV1 I-589) and/or 454 and 455 (AAV1 I-454), 455 and 456 (AAV1 I-455) or 456 and 457 (AAV1 I-456) having the amino acid sequence of SEQ ID NO: 1;
   (c) the insertion site is between two amino acids corresponding to amino acid position 588 and 589 (AAV8 I-588) or 589 and 590 (AAV8 I-589) and/or 455 and 456 (I-455), 456 and 457 (I-456) or 457 and 458 (I-457) of AAV8 VP1 having the amino acid sequence of SEQ ID NO: 8, or
   (d) the insertion site is between two amino acids corresponding to amino acid position 588 and 589 (AAV9 I-588) or 589 and 590 (AAV9 I-589) and/or 454 and 455 (I-454), 455 and 456 (I-455) or 456 and 457 (I-456) of AAV9 VP1 having the amino acid sequence of SEQ ID NO: 9.
23. The AAV or AAVLP according to any one of items 17-22, wherein the AAV or AAVLP has a capsid of about 60 VPs, wherein the VPs are
   (a) VP3;
   (b) VP1 and VP3; or
   (c) VP1, VP2 and VP3 proteins, preferably at a ratio of 1:1:10.
24. The AAV or AAVLP according to any one of items 17-23, wherein the insert is a protein comprising a binding domain and wherein the AAV comprises an ITR-flanked genome and is infectious, and wherein the ITR-flanked genome comprises a transgene.
25. The AAV or AAVLP according to any one of items 17-24, wherein the insert is a protein comprising a binding domain specific for a binding target and wherein the protein comprising a binding domain determines the tropism of the AAV or AAVLP for a target cell expressing the binding target on its surface.
26. The AAV or AAVLP according to any one of items 17-25, wherein the AAV or AAVLP is retargeted.
27. The AAV or AAVLP according to any one of the preceding items 17-26, wherein the AAV or AAVLP is formed by 2 or more viral proteins comprising different inserts of a least about 75-400 amino acids, preferably about 75-300 amino acids, wherein the first insert comprises a first protein comprising a binding domain and the at least one further insert comprises a further protein comprising a binding domain and/or an immunogenic protein or a portion thereof.
28. The AAV or AAVLP according to any one of items 17-27, wherein the insert is a protein comprising an antigen-binding domain.
29. The AAV or AAVLP according to item 28, wherein the insert is a protein comprising an antigen-binding domain specific for a target antigen and wherein the antigen-binding domain determines the tropism of the AAV or AAVLP for a target cell expressing the target antigen in its surface.
30. The AAV or AAVLP according to any one of items 25 to 27, wherein the AAV or AAVLP is formed by 2 or more viral proteins comprising different inserts of a least about 75-400 amino acids, preferably about 75-300 amino acids, wherein the first insert is a protein comprising an antigen-binding domain specific for a first target antigen and the at least one further insert is a protein comprising an antigen-binding domain specific for further target antigen, or an immunogenic protein or a portion thereof.
31. The AAV or AAVLP of any one of items 28-30, wherein the protein comprising an antigen-binding domain is a single-domain antibody (sdAb), a single chain variable fragment (scFv) or an antibody mimetic (e.g., an anticalin, an affibody, an adnectin, a monobody, a DARPin, an affimer, or an affitin).
32. A pharmaceutical composition comprising the AAV or AAVLP according to of any one of items 17-31, further comprising at least one pharmaceutically acceptable excipient.
33. An AAV or AAVLP according to any one of items 17 to 31 for use in therapy.
34. An AAV or AAVLP according to any one of items 17 to 31 for use in gene therapy.
35. A pharmaceutical composition according to item 32 for use in therapy, preferably gene therapy
36. The AAV or AAVLP for use according to items 33 or 34, or the pharmaceutical composition for use according to item 35, wherein the AAV or AAVLP or the pharmaceutical composition is to be administered via the intranasal mucosal, sublingual, oral, buccal, intravenous, intramuscular, intraperitoneal or subcutaneous route, preferably wherein the AAV or AAVLP is to be administered by inhalation via the intranasal, oral and/or mucosal route.
37. A method for producing an AAV or an AAVLP, comprising the steps of
   (i) preparing a cell comprising at least one DNA sequence comprising a cap gene and a rep gene, at least one DNA sequence comprising adenoviral helper sequences and optionally at least one DNA sequence comprising an ITR-flanked genome;
      wherein the cap gene encodes a protein comprising an insert of about 75-400 amino acids, preferably about 75-300 amino acids, in the viral proteins (VPs) forming the capsid at an insertion site (I) at the top of variable region VIII and/or variable region IV (VR-VIII and/or VR-IV) of the VPs, and wherein the insert is optionally flanked by a linker comprising one or more amino acids on both sides, preferably selected from the group consisting of A (Ala), G (Gly), S (Ser), T (Thr), L (Leu) and combinations thereof;
   (ii) cultivating the cells under conditions allowing the production of the AAV or the AAVLP;
   (iii) purifying the AAV or the AAVLP; and
      optionally adding at least one pharmaceutically acceptable excipient to formulate the AAV or the AAVLP into a pharmaceutical composition.
38. A method for producing a pharmaceutical composition comprising an AAV or an AAVLP, comprising the steps of
   (i) preparing a cell comprising at least one DNA sequence comprising a cap gene and a rep gene, at least one DNA sequence comprising adenoviral helper sequences and optionally at least one DNA sequence comprising an ITR-flanked genome;
      wherein the cap gene encodes a protein comprising an insert of about 75-400 amino acids, preferably about 75-300 amino acids, in the viral proteins (VPs) forming the capsid at an insertion site (I) at the top of variable region VIII and/or variable region IV (VR-VIII and/or VR-IV) of the VPs, and wherein the insert is optionally flanked by a linker comprising one or more amino acids on both sides, preferably selected from the group consisting of A (Ala), G (Gly), S (Ser), T (Thr), L (Leu) and combinations thereof;
   (ii) cultivating the cells under conditions allowing the production of the AAV or the AAVLP;
   (iii) purifying the AAV or the AAVLP; and
   (iv) adding at least one pharmaceutically acceptable excipient to formulate the AAV or the AAVLP into a pharmaceutical composition.
39. The method of item 37 or 38, wherein the insert is a protein comprising a binding domain, preferably a protein comprising an antigen-binding domain.

### EXAMPLES

### Example 1: Structural Models of AAV Capsid and AAV Viral Protein (VP1)

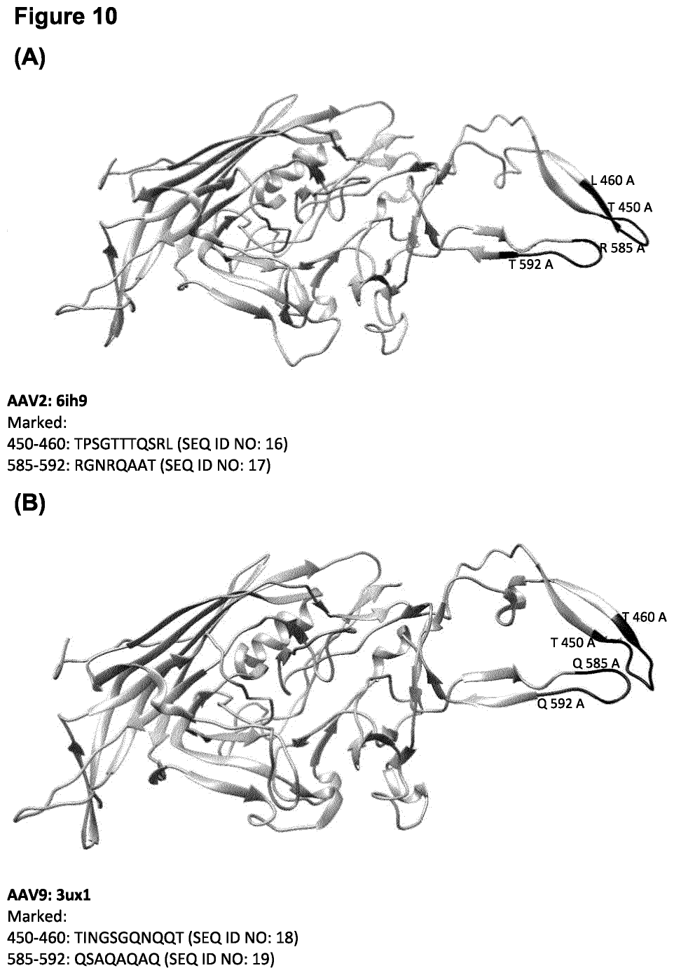

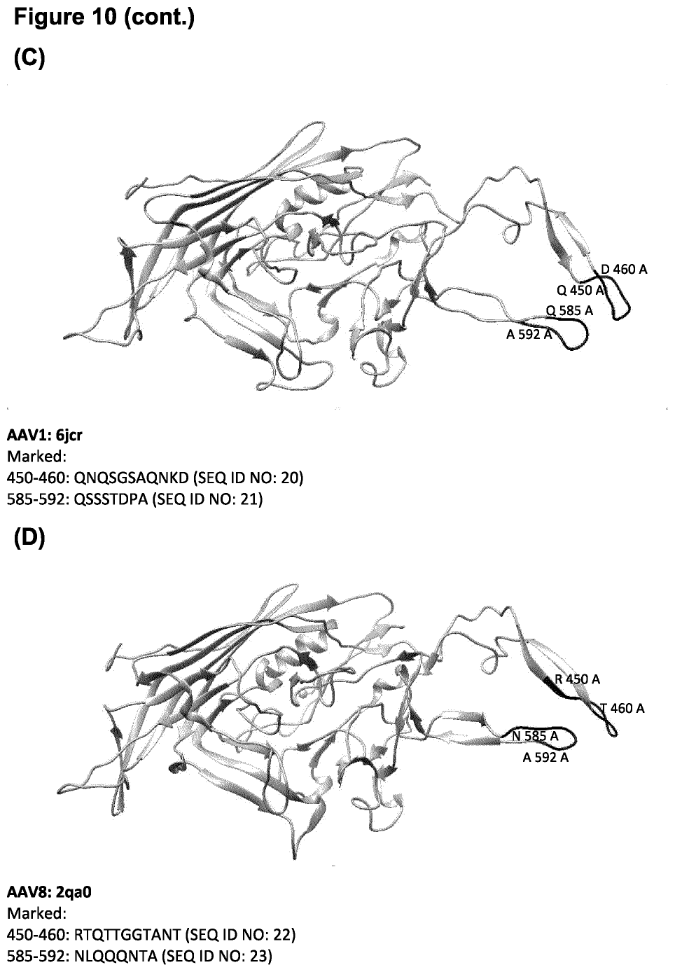

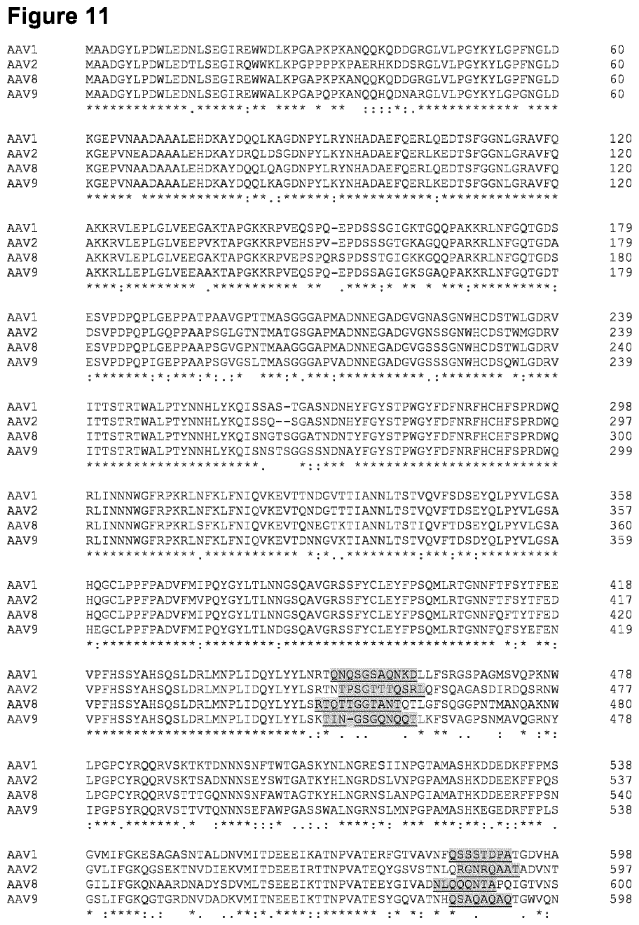

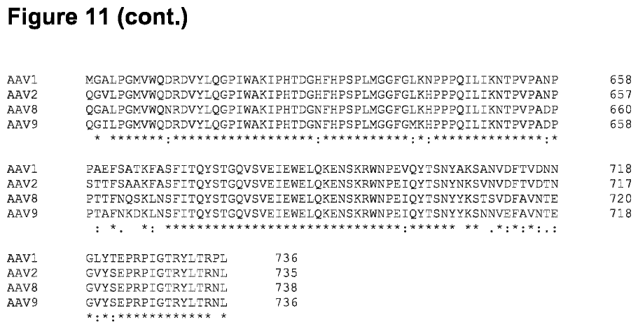

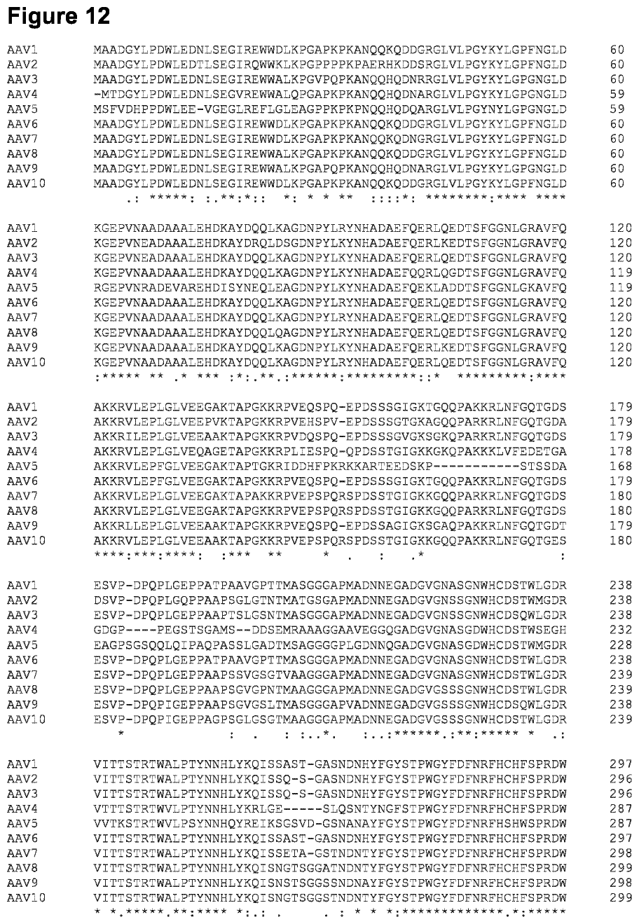

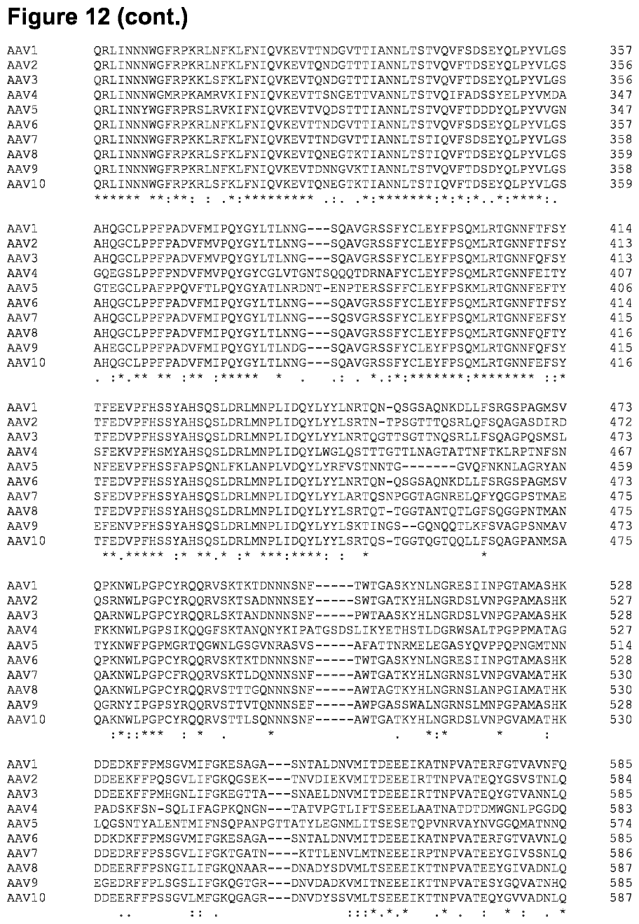

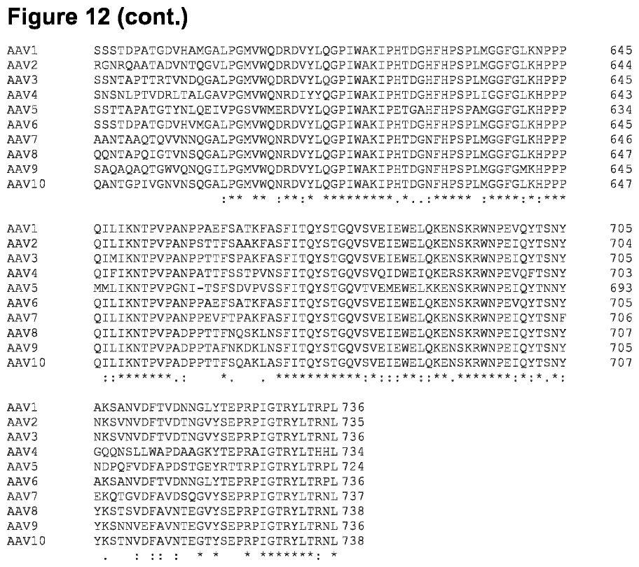

In order to analyse whether a large protein, such as the spike (S) protein of SARS-CoV-2, can be introduced into VP1 of AAV, the structure of VP3 of AAV2 (SEQ ID NO:2) and the resulting AAV capsid of AAV2 comprising the spike receptor binding domain of the S protein of SARS-CoV-2 (HtW2_S1.1) (insert: SEQ ID NO: 11, AAV2 VP1 with insert: SEQ ID NO: 13) has been modelled. The comparative structural modelling was performed using Robetta protein structure prediction service (https://robetta.bakerlab.org/) for HtW2_S1.1 VP3 (FIG. 1B) based on AAV2 WT (PDB 6ih9) (FIG. 10) and was processed using Chimera software (https://www.cgl.ucsf.edu/chimera/). The same protein was further modelled using RoseTTAFold (https://robetta.bakerlab.org/) based on de novo prediction of protein structures of VP3 of AAV2 comprising the spike receptor binding domain of the S protein of SARS-CoV-2 (HtW2_S1.1) as insert at I-587 (FIG. 1D). The published structure based on AAV2 WT (PDB 6ih9) is shown in the same orientation in FIG. 1E. The corresponding predicted 60-mer capsid structure of HtW2_S1.1 (novel AAV variant HtW2_S1.1 with an insertion of 202 amino acids comprising a portion of the SARS-CoV-2 S1 spike flanked by linker amino acids) was analysed from two distinct angles as shown in FIG. 1F and 1E. As may be taken from FIGS. 1B, D, E and G, the large >200 amino acid insert did not compromise the principle gross VP1 or capsid structure.

### Example 2: Cloning of pHtW2 S1.1 and pHtW9 S1.1 AAV Rep/Cap Plasmids and Preparation of HtW2 S1.1 Full Particles and HtW2 S1.1 Empty Particles

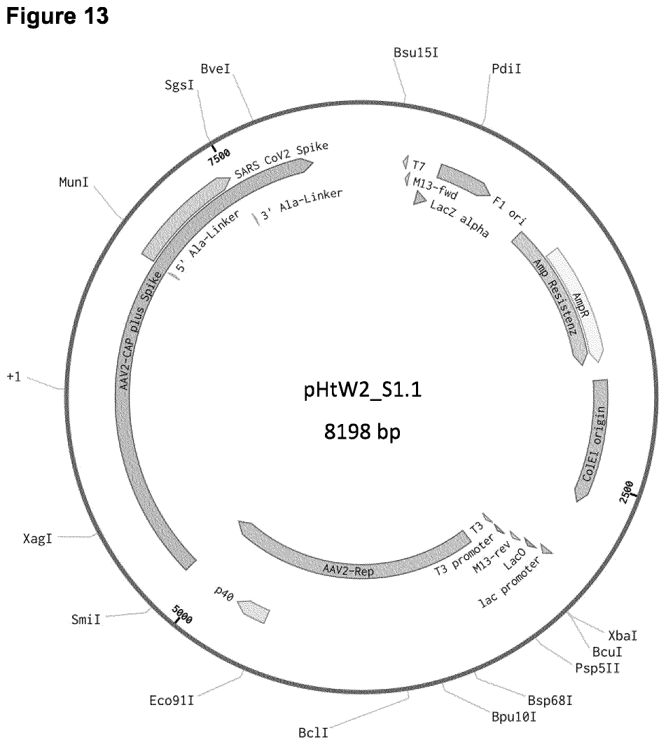

The RBD-spike-sequence having the SEQ ID NO: 11 was PCR amplified with pHtW2_S1.1_fw and pHtW2_S1.1_rv primers (see Table 3). The PCR product was run on an agarose gel, the 567 bp amplicon excised and purified (QiaQuick Gel Extraction Kit, Qiagen) and used for a Gibson assembly reaction with the 7590 bp Mlul/Sgsl double-digested and agarose gel-purified pRC'99 plasmid containing AAV2 Rep and Cap sequences. Successful Gibson assembly yielded the 8198 bp AAV2 Rep/HtW2_S1.1 Cap pHtW2_S1.1 helper plasmid depicted in FIG. 13.

For cloning the pHtW9_S1.1 version, pAAV2/9 Cap plasmid (7390 bp) was used as a template for a PCR reaction with primers pHtW9_S1.1_BB_fw and pHtW9_S1.1_BB_ry (see Table 3), the 7390 bp amplicon was agarose gel-purified and used as the linearized backbone for the Gibson assembly reaction.

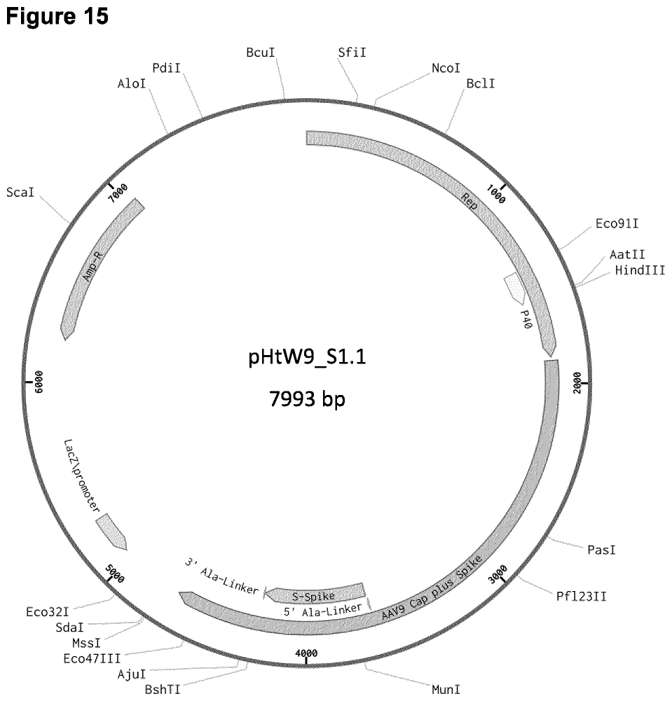

The RBD-spike-sequence having the SEQ ID NO: 11 was PCR amplified with pHtW9_S1.1_fw and pHtW9_S1.12v primers (see Table 3). The resulting 654 bp amplicon was agarose gel-purified (QiaQuick Gel Extraction Kit, Qiagen) and used for the Gibson assembly reaction which yielded the 7993 bp AAV2 Rep/HtW9_S1.1 Cap pHtW9_S1.1 plasmid depicted in FIG. 15. Correct assembly of the plasmids was confirmed with Sanger sequencing.

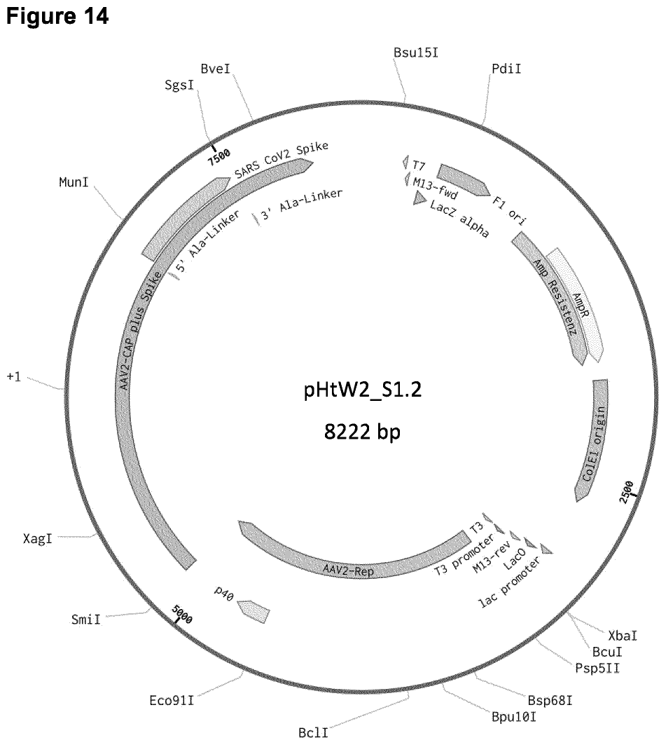

The RBD-spike-sequence encoding the SEQ ID NO: 69 was PCR amplified with HtW2_Var_S1.2_fw and HtW2_Var_S1.22v primers (see Table 3). The PCR product was run on an agarose gel, the 685 bp amplicon excised and purified (QiaQuick Gel Extraction Kit, Qiagen) and used for a Gibson assembly reaction with the 7590 bp Mlul/Sgsl double-digested and agarose gel-purified pRC'99 plasmid containing AAV2 Rep and Cap sequences. Successful Gibson assembly yielded the 8222 bp AAV2 Rep/HtW2_S1.2 Cap helper plasmid pHtW2_S1.2 depicted in FIG. 14.

TABLE 3
Cloning primers for construction of pHtW2_S1.1, pHtW2_S1.2
and pHtW9_S1.1 plasmids.

| Name | SEQ ID NO: | Sequence (5′-3′) |
|---|---|---|
| pHtW2_S1.1_fw | SEQ ID NO: 24 | CTACCAACCTCCAGAGAGGCAACGCGGCCG |
| | | CAACTAATCTTTGTCCGTTCGGTGAGGTTT |
| pHtW2_S1.1_rv | SEQ ID NO: 25 | TGACATCTGCGGTAGCTGCTTGGCGCGCCG |
| | | CTCCCTTTTTGGGCCCACAAACTGT |
| pHtW9_S1.1_fw | SEQ ID NO: 26 | CCACAAACCACCAGAGTGCCCAAGCGGCCG |
| | | CAACTAATCTTTGTCCGTTCGGTGAGGTTT |
| pHtW9_S1.1 rv | SEQ ID NO: 27 | TTTGAACCCAACCGGTCTGCGCCTGTGCCG |
| | | CCTTTTTGGGCCCACAAACTGTAGC |
| pHtW9_S1.1_BB_fw | SEQ ID NO: 28 | GCACAGGCGCAGACCGGTTGGGTTCAAAAC |
| | | CAA |
| pHtW9_S1.1_BB_rv | SEQ ID NO: 29 | TTGGGCACTCTGGTGGTTTGTGG |
| HtW2_Var_S1.2_fw | SEQ ID NO: 71 | TATCTACCAACCTCCAGAGAGGCAACGCGG |
| | | CGGCGAAGTGCACCCTGAAGAGCTTCACC |
| HtW2_Var_S1.2_rv | SEQ ID NO: 71 | TTGACATCTGCGGTAGCTGCTTGGCGCGCC |
| | | GCGTATCCCACTCCGTTGGTTGGCT |

The pHtW2_S1.1 or pHtW9_S1.1 plasmid was used to produce AAV particles with the novel AAV capsid variant comprising the RBD-spike-sequence having the amino acid sequence of SEQ ID NO: 11. The resulting novel HtW2_S1.1 cap protein has the amino acid sequence of SEQ ID NO: 13. The pHtW2_S1.2 plasmid was used to produce AAV particles with the novel AAV capsid variant comprising the spike-sequence (comprising the binding domain and additional T cell epitopes at the N-terminal end) having the amino acid sequence of SEQ ID NO: 69. The resulting novel HtW2_S1.2 cap protein has the amino acid sequence of SEQ ID NO: 70. AAV production was performed by standard techniques described in Michalakis et al. (Mol. Ther. (2010); 18(12): 2057-2063). In brief, AAVs were produced by transfection of HEK293T cells cotransfected with equimolar amounts of pHtW2_S1.1, pHtW2_1.2 or pHtW9_S1.1, optionally a self-complementary (sc) AAV cis plasmid (pTransgene plasmid) containing a CMV-eGFP expression cassette (AAV-sc-CMV-eGFP) (Hacker et al. (2005) “Adeno-associated virus serotypes 1 to 5 mediated tumor cell directed gene transfer and improvement of transduction efficiency.” J Gene Med 7(11):1429-38) and an adenoviral helper plasmid (e.g. pXX6 from J. Samulski, Chapel Hill, NC; Xiao, Li and Samulski (1998) “Production of high-titer recombinant adeno-associated virus vectors in the absence of helper adenovirus.” J. Virol. 72: 2224-2232) for packaging. HtW2_S1.1 empty particles were produced in the absence of the pTransgene plasmid (carrying an ITR-flanked sc-CMV-eGFP expression cassette). For the production of AAVs, 15 150-mm petri dishes of HEK293T cells at 80% confluence were cotransfected with 20 μg of DNA per petri dish. The pHtW2_S1.1, pHtW2_S1.2 or pHtW9_S1.1 AAV Rep/Cap plasmid was thereby cotransfected at equimolar ratio with an adenoviral helper plasmid (e.g. pXX6 from J. Samulski, Chapel Hill, NC; Xiao, Li and Samulski (1998) “Production of high-titer recombinant adeno-associated virus vectors in the absence of helper adenovirus.” J. Virol. 72: 2224-2232) and in case of production of full capsids with equimolar amount of a pTransgene plasmid containing an ITR-flanked CMV-eGFP cassette. After 48 h AAVs were isolated from HEK293 cell pellets which were resuspended in 150 mM NaCl, 50 mM Tris—HCl (pH 8.5), freeze—thawed several times, and treated with Benzonase (50 U/ml) for 30 min at 37° C. Cell debris was removed by centrifugation, and supernatant was further processed for iodixanol gradient. Alternatively, AAVs were isolated from cell culture supernatants after overnight precipitation at 4° C. with 8% polyethylene glycol (PEG) 8000. The PEG-AAV precipitate was then treated with Benzonase (50 U/ml) for 30 min at 37° C. and further processed for iodixanol gradient. Iodixanol gradient ultracentrifugation was performed at 70,000 rpm for 1 h and 45 min at 18° C. as described (Zolotukhin et al. (1999) “Recombinant adeno-associated virus purification using novel methods improves infectious titer and yield. Gene Ther. 6: 973-985). Virions were then harvested from the 40% iodixanol phase and titrated by real-time PCR, carried out with the Step one Plus (Thermo Fisher scientific, Germany) using the AAV2 free ITR qPCR assay described in D'Costa et al., (2016) Practical utilization of recombinant AAV vector reference standards: focus on vector genomes titration by free ITR qPCR, 5:16019.

### Example 3: AAVx Affinity Purification Chromatography of AAV Vectors

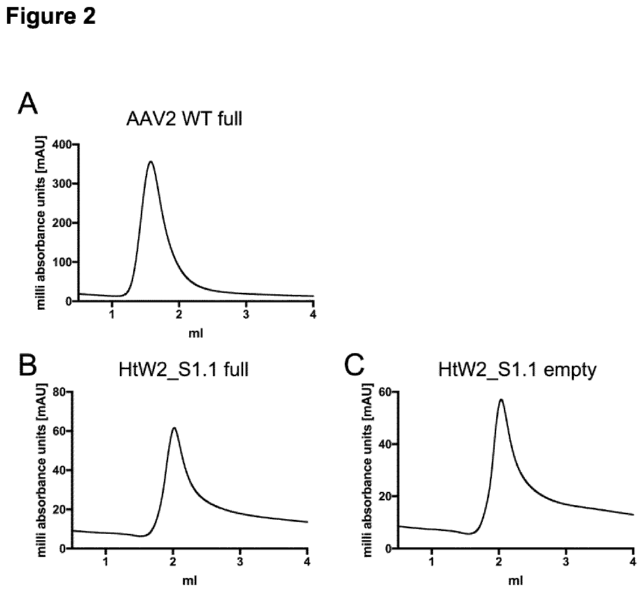

AAV2 WT particles, HtW2_S1.1 full particles, i.e. full AAV particles (HtW2_S1.1 particles loaded with sc-CMV-eGFP genomes), and HtW2_S1.1 empty particles, i.e. empty AAV particles (HtW2_S1.1 particles produced in the absence of the pTransgene plasmid (carrying an ITR-flanked sc-CMV-eGFP expression cassette)) were purified using a Poros Capture Select AAVx affinity purification column (obtained from Thermo Fisher Scientific) according to the manufacturer's instructions. The chromatograms of FIG. 2 show the elution of AAV2 WT particles (FIG. 2A), HtW2_S1.1 full particles (FIG. 2B) as well as HtW2_S1.1 empty particles (FIG. 2C). HtW2_S1.1 full and empty particles bound to the AAVx affinity purification column and were eluted at a similar but slightly delayed time after initiation of the elution process (indicated by the ml values of elution buffer on the x-axis). Thus, despite the large insertion of more than 200 amino acids, both HtW2_S1.1 full and HtW2_S1.1 empty particles still retained the ability to bind to the AAVx affinity purification column. HtW2_S1.2 and HtW9_S1.1 have been purified similarly.

### Example 4: Transduction Assay of AAV Vectors in HeLa Cells

Native HeLa cells were transduced with different multiplicities of infection ((MOI): 250 MOI, 500 MOI and 1000 MOI) of AAV-sc-CMV-eGFP packaged with AAV2 WT or the novel AAV variant HtW2_S1.1 with an insertion of 202 amino acids comprising part of the SARS-CoV-2 S1 spike RBD having the amino acid sequence of SEQ ID NO: 11 flanked by linker amino acids. The cells were imaged after 24 h and 48 h and analysed using brightfield and epifluorescence microscopy (Evos FL, Thermo Fisher Scientific). After imaging at 48 h, the cells were collected and the fraction of eGPF-positive cells was analysed using a Countess II FL Automated Cell Counter (Thermo Fisher Scientific).

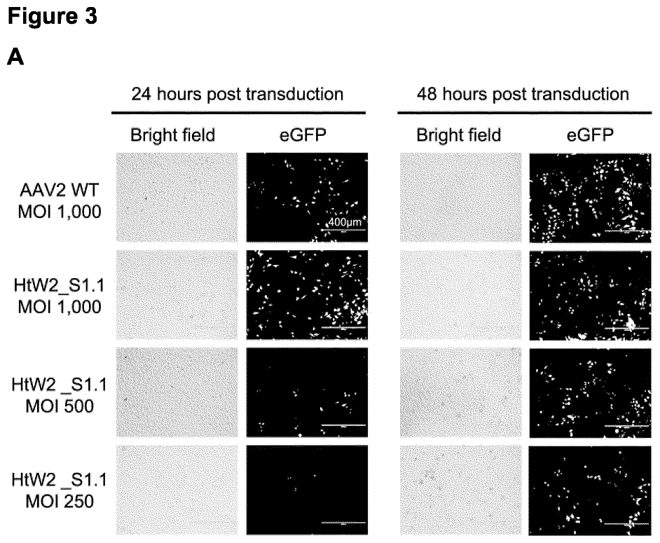

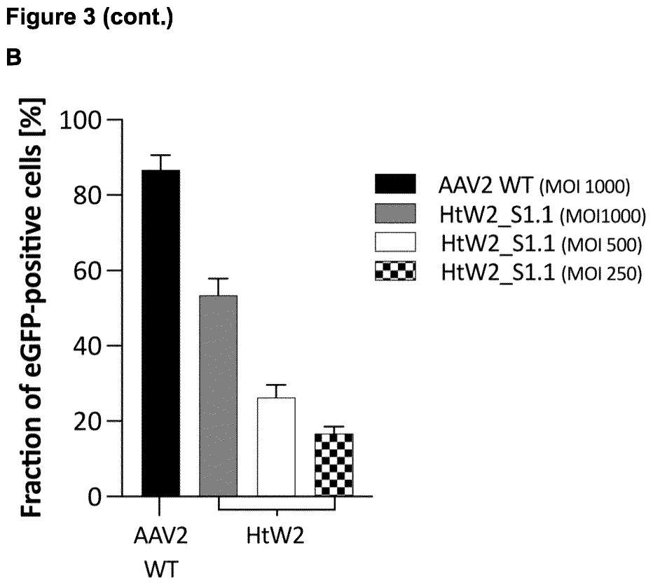

Surprisingly, as may be taken from FIG. 3, despite large insertion of more than 200 amino acids, HtW2_S1.1 still retained the ability to infect and transduce human cells even at very low MOI 250.

For overexpression of the ACE2 receptor in HeLa cells, native HeLa cells were transiently transfected with a plasmid comprising the amino acid sequence of SEQ ID NO: 30 under the control of a CMV promoter. After 48 h the HeLa cells were transduced with two different MOIs (250 MOI and 500 MOI) of AAV-sc-CMV-eGFP packaged with the novel AAV variant HtW2_S1.1 with an insertion of 202 amino acids comprising part of the SARS-CoV-2 S spike RBD having the amino acid sequence of SEQ ID NO: 11 flanked by linker amino acids. The cells were imaged after 24 h and 48 h using epifluorescence microscopy (Evos FL, Thermo Fisher Scientific) (FIG. 4A). After imaging at 48 h, the cells were collected and the fraction of eGPF-positive cells was analysed using a Countess II FL Automated Cell Counter (Thermo Fisher Scientific) (FIG. 4B).

Interestingly, as may be taken from FIG. 4, insertion of 202 amino acids comprising part of the SARS-CoV-2 S spike RBD having the amino acid sequence of SEQ ID NO: 11 endowed particles with higher infectivity and transduction efficiency of HeLa cells transfected with ACE2. This confirms that the AAV was successfully repurposed to behave like SARS-CoV-2 in terms of cellular tropism, implying that the incorporated SARS-CoV-2-derived protein sequences is correctly folded and confers immunogenic properties of SARS-CoV-2 to the AAV particle. Thus, these data suggest that the HtW2_S1.1 has a SARS-CoV-2-like tropism and shows higher infectivity of ACE2-overexpressing human cells.

### Example 5: Transduction Assay of AAV Vectors in HEK293T Cells Stably Transfected with ACE2

HEK293T cells were stably transfected with ACE2 and transduced with HtW2_S1.2 vectors. Representative epifluorescence images from native (left column, −ACE2) or stable ACE2-overexpressing (right column, +ACE2) HEK293T cell cultures at 48 hours (right panels) after transduction with MOI 250 (upper row), MOI 500 (middle row) or MOI 1000 (bottom row) of AAV-sc-CMV-eGFP packaged with the novel AAV variant HtW2_S1.2 with an insertion of 206 amino acids comprising part of the SARS-CoV-2 S1 spike protein (SEQ ID NO: 69) are shown in FIG. 5A. FIG. 5B shows the fraction of eGFP-positive cells measured with Countess II FL Automated Cell Counter in native or stable ACE2-overexpressing HEK293T cell cultures at 48 hours after transduction with MOI 250, 500 and 1000 of AAV-sc-CMV-eGFP packaged with the novel AAV variant HtW2_S1.2 with an insertion of 206 amino acids comprising the binding domain of the SARS-CoV-2 S1 spike protein flanked by linker amino acids. Data were analysed using 1-way ANOVA, S̆idák's multiple comparisons test. The data shown in FIG. 5 confirm that also HtW2_S1.2 has a SARS-CoV-2-like tropism and shows higher infectivity of ACE2-overexpressing human cells.

### Example 6: Immunogenicity of AAV Vectors Comprising a Portion of the SARS-CoV-2 Spike Protein as Insert in Rabbits

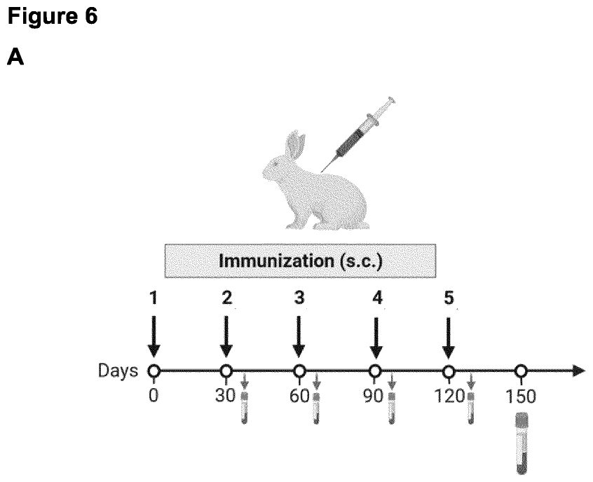

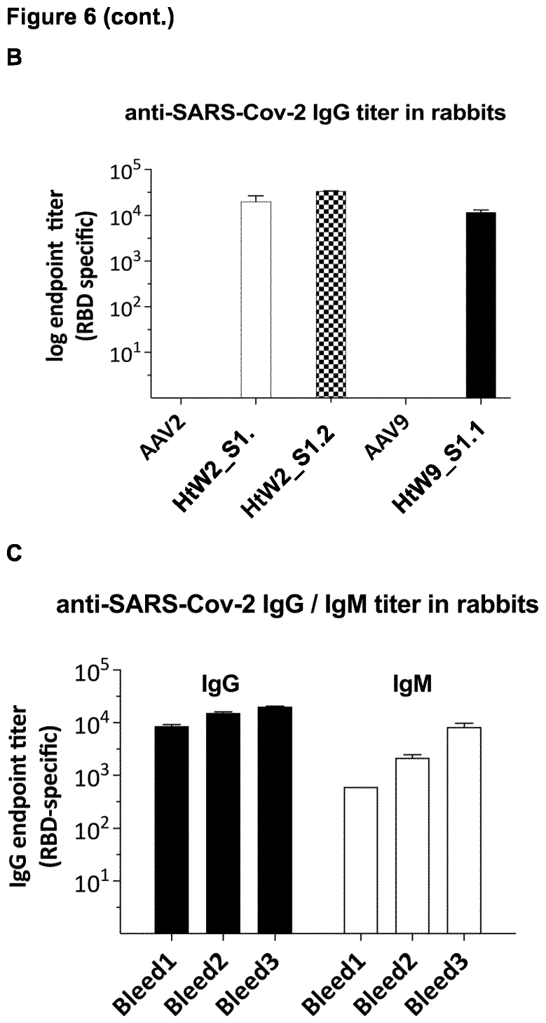

Humoral response to HtW were evaluated in rabbits. Rabbits (Zika, 12 weeks old, female) were injected with 15 μl of wildtype AAV empty capsids (AAV2 WT, AAV9 WT) or HtW empty capsids (HtW2_S1.1, HtW2_S1.2 or HtW9_S1.1) subcutaneously at approximately 7.5×10⁸ capsid particles (cp)/μl. All animal experiments were handled in compliance with the European and national regulations for animal experimentation (European Directive 2010/63/EU; Animal Welfare Acts in Germany) and were performed by a private service provider. For primary immunization, supernatants were emulsified in Freund's complete adjuvant (Sigma-Aldrich, #344289), booster injections were at 4 week intervals (day 30, 60, 90 and 120, s.c.) with Freund's incomplete adjuvant (Sigma-Aldrich, #F5506). Bleedings were taken 10 days following each booster injection (i.e., at days 40, 70, 100, and 130 following the first injection) and the last bleed was taken 150 days following the first injection (FIG. 6A).

Immunogenicity of HtW capsids was assessed in rabbits immunized with wildtype AAV empty capsids (AAV2 WT, AAV9 WT) or HtW empty capsids (HtW2_S1.1, HtW2_S1.2 or HtW9_S1.1) by ELISA from blood taken 10 days after the second booster injection. Sera were separated by centrifugation at 1,200 g for 20 min and SARS-CoV-2 specific IgG titers were determined by ELISA using recombinant RBD (Acro Biosystems, #SPD-C52H2) as the antigen. Antisera titers were determined as described in Frey A et al., J Immunol Methods, 1998, 221(1-2):35-41. The IgG endpoint titers against SARS-CoV-2 wild type RBD are shown in FIG. 6B. Minimum amounts of HtW empty capsids that are below the detection limit of silver stain induce a strong immune response in rabbits. No SARS-CoV-2 RBD-specific IgG signal was elicited by AAV2 and AAV9 WT (FIG. 6B).

Further, rabbit sera collected 10 days after the first (Bleed1), second (Bleed2) and third (Bleed3) booster injection with the empty AAV vectors were analysed regarding the endpoint antibody titer. Titers were determined by ELISA using SARS-CoV-2 RBD as antigen (Acro Biosystems, # SPD-C52H2). Antibody response was determined using peroxidase labelled anti-IgG (Abcam, # ab6721) and anti-IgM secondary antibodies (Abcam, #97195). Endpoint titers of SARS-CoV-2 RBD specific IgG and IgM antibodies are shown in FIG. 6C. HtW2 S1.2 expressed SARS-CoV-2 wild type RBD induces a strong and sustained humoral IgG response already after the first booster injection. IgM titers are weaker but increase with booster injections.

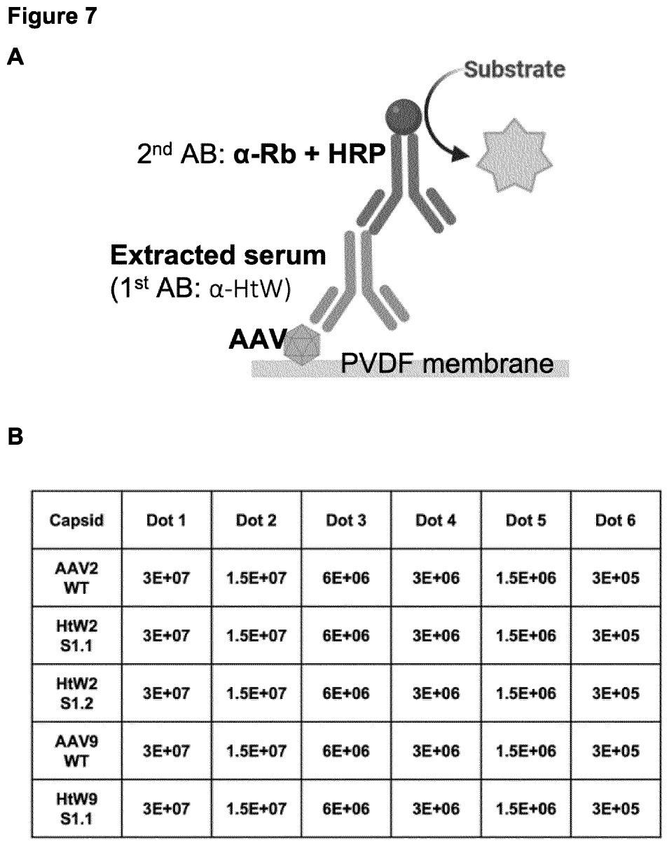

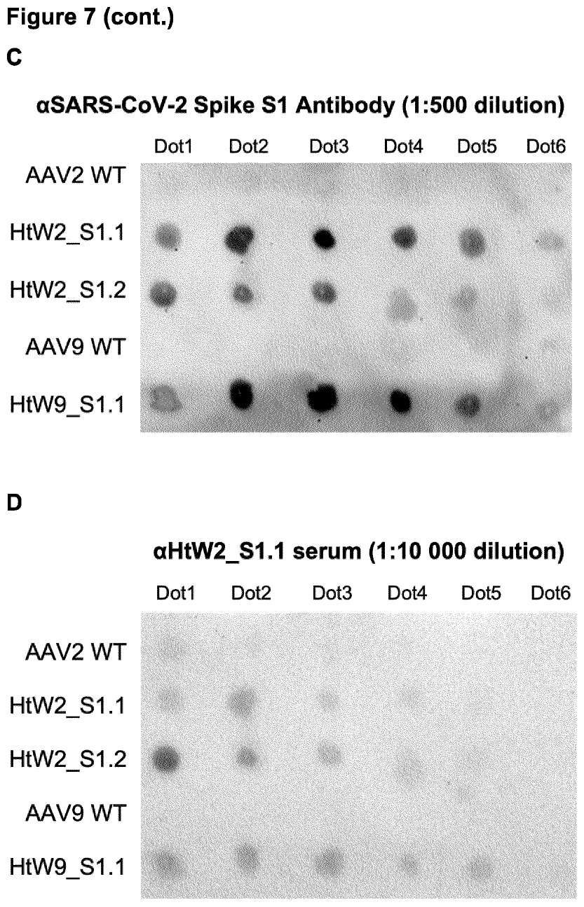

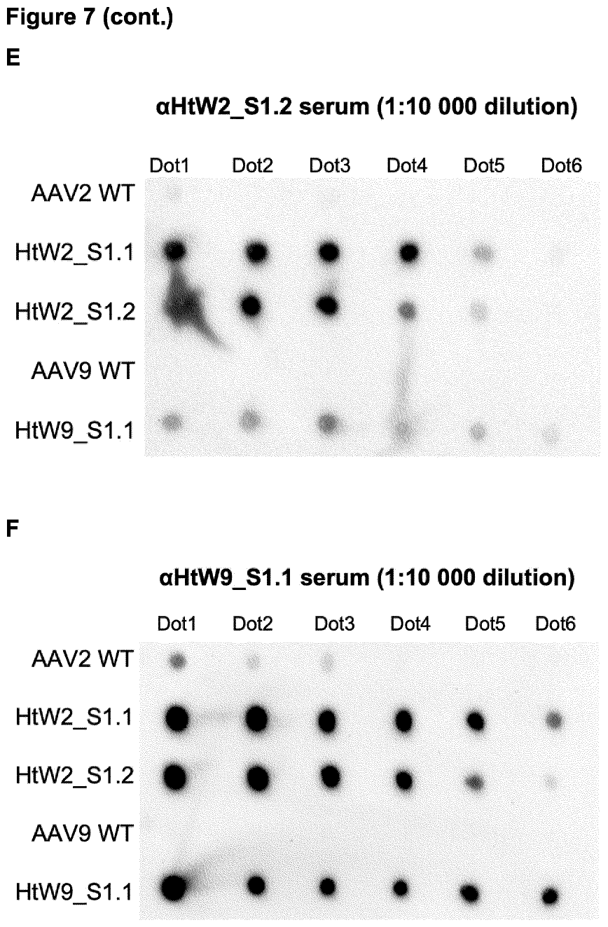

To further evaluate immunogenicity of HtW2_S1.1, HtW2_S1.2 and HtW9_S1.1 dot blot assays of AAV vectors at various titers spotted on polyvinylidene difluoride (PVDF) membranes and stained with a commercial antibody and rabbit sera (final bleeding) was performed. The method is schematically depicted in FIG. 7A. The PVDF membrane is activated using 100% MeOH and incubated in TBS-T buffer. The AAV vector is spotted from 3×10⁷ to 3×10⁵ total vector genomes/dot (FIG. 7B) onto the PVDF membrane and allowed to air dry. Subsequently, the membrane is blocked with 5% dry milk powder in TBST, washed and then incubated for 1 hour at room temperature (RT) with the corresponding serum dilution in 1% dry milk powder in TBST. After washing in TBST, the membrane is incubated for another hour at RT with HRP-conjugated secondary antibody, followed by washing and standard luminescence reaction and detection. Specifically, the dot blot was labelled with rabbit monoclonal anti-SARS-CoV-2 Spike S1 antibody from Sino Biological (αSARS-CoV-2 Spike S1, Cat: 40150-R007) at 1:500 dilution (FIG. 7C) or anti-HtW2_S1.1, anti-HtW2_S1.2 or anti-HtW9_S1.1 serum (αHtW2_S1.1, αHtW2_S1.2 or αHtW9_S1.1) at 10.000 dilution (FIG. 7D-E).

Using the commercial control antibody, no signal was obtained with AAV2 wildtype (WT) and AAV9 WT, while HtW2_S1.1, HtW2_S1.2 and HtW9_S1.1 all gave strong immunosignals up to the second lowest vector amount spotted on the PVDF membrane (e.g. dot 5 with 1.5×10⁶ total vector genomes (1.5E6)) (FIG. 7C). All rabbit sera following immunization with HtW2_S1.1, HtW2_S1.2 or HtW9_S1.1 could be used at high dilutions. Results from the dot blot labelled with a 1:10000 dilution of a serum (αHtW2_S1.1) from a rabbit which was immunized with HtW2_S1.1 empty capsid are shown in FIG. 7D. A signal was obtained on dots spotted with HtW2_S1.1, HtW2_S1.2 or HtW9_S1.1. No signal was obtained with AAV9 WT and only weak signal was seen with AAV2 WT. Results from the dot blot labelled with a 1:10,000 dilution of a serum (αHtW2_S1.2) from a rabbit which was immunized with HtW2_S1.2 empty capsid are shown in FIG. 7E. Very intense signal was obtained with both HtW2_S1.1 and HtW2_S1.2. Weaker signal, but with higher dilution was seen with HtW9_S1.1. No signal was obtained with AAV2 WT and AAV9 WT. Results from the dot blot labelled with a 1:10,000 dilution of a serum (αHtW9_S1.1) from a rabbit which was immunized with HtW9_S1.1 empty capsid are shown in FIG. 7F. Very intense signal was obtained with both HtW2_S1.1, HtW2_S1.2 and HtW9_S1.1. No signal was obtained with AAV9 WT and only weak signal was seen with AAV2. These results confirm the presence of the SARS-CoV-2 Spike S1 sequence on the novel HtW capsid and show that all HtW variants elicit a strong SARS-CoV-2-specific humoral immune response. A clear cross-reactivity among the variants was observed, confirming that the immune response is directed against the inserted sequence and not the AAV capsid backbone.

### Example 7: Evaluation of Serum from Comirnaty Immunized HtW Engineered Capsids

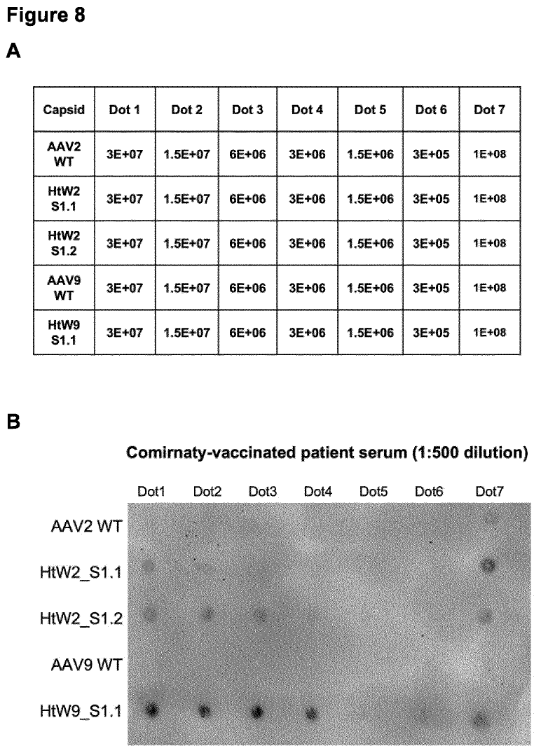

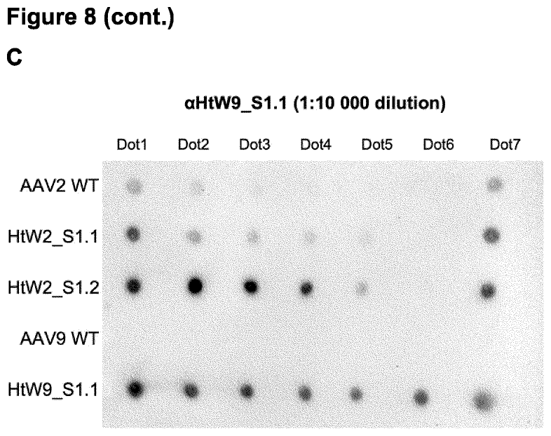

AAV vectors spotted on PVDF membranes at 3×10⁷ to 3×10⁵ and additionally 1×10⁸ total vector genomes as indicated in FIG. 8A were stained in dot blots with Cormirnaty-vaccinated patient serum. Dot blots were labelled with a 1:500 dilution of a serum from a patient collected 1 weeks after the second vaccination with Comirnaty (BNT162b2, Biontech/Pfizer). A specific signal was obtained on dots spotted with HtW2_S1.1, HtW2_S1.2 or HtW9_S1.1. The signal was strongest on HtW9_S1.1. No signal was obtained with AAV9 WT and only very faint signal was seen with AAV2 WT, which is not surprising given that up to 80% of the general population is seropositive for AAV2. (C) The same dot blot, which was stripped and re-labelled with a 1:10000 dilution of a serum (αHtW9_S1.1) from a rabbit which was immunized with HtW9_S1.1 empty capsid. Intense to very intense signal was obtained with all three HtW variants (HtW2_S1.2 and HtW9_S1.1, and weaker with HtW2_S1.1). No signal was obtained with AAV9 WT and only weak signal was seen with AAV2 WT. These results confirm that antibodies elicited in humans in response to the authorized mRNA-vaccine Comirnaty (BNT162b2) cross-react with all three HtW engineered capsids.

In an independent experiment human PBMCs stimulated with HtWs demonstrated activation of various immune cells, including T cells (CD3+, CD4+ and CD8+) B cells (CD19+) and NK cells (CD56+) as indicated by the activation marker CD69 as well as increased cell numbers of subtypes indicative for an activated state. This further confirms that HtWs are highly immunogenic and may potentially be used as vaccine (primary and/or booster) for preventing or treating SARS-CoV-2 infections.

### Example 8: Neutralization Assay with Serum of HtW9 S1.1-Immunized Rabbits in HEK293T Cells Stably Expressing ACE2 (HEK293T+ACE2).

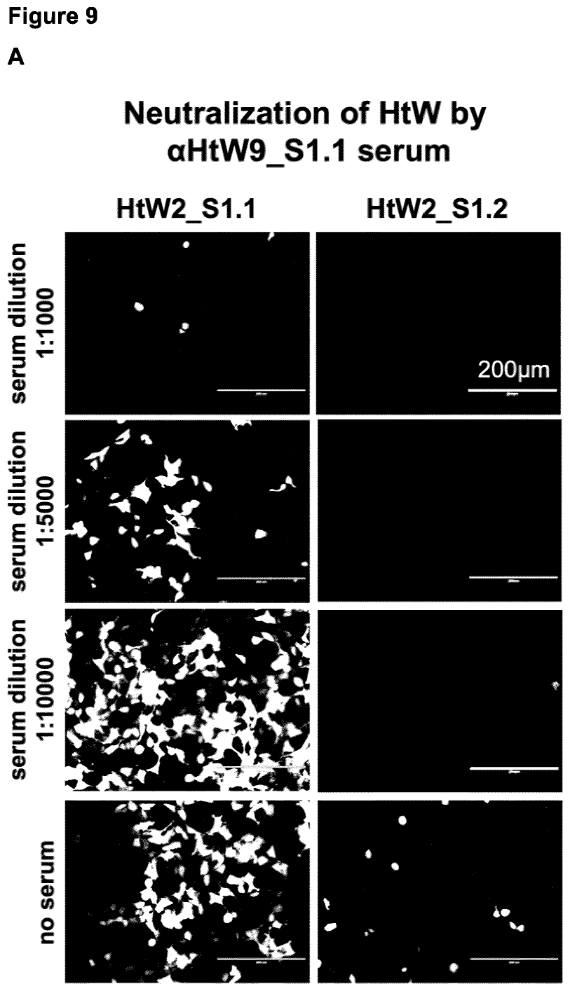

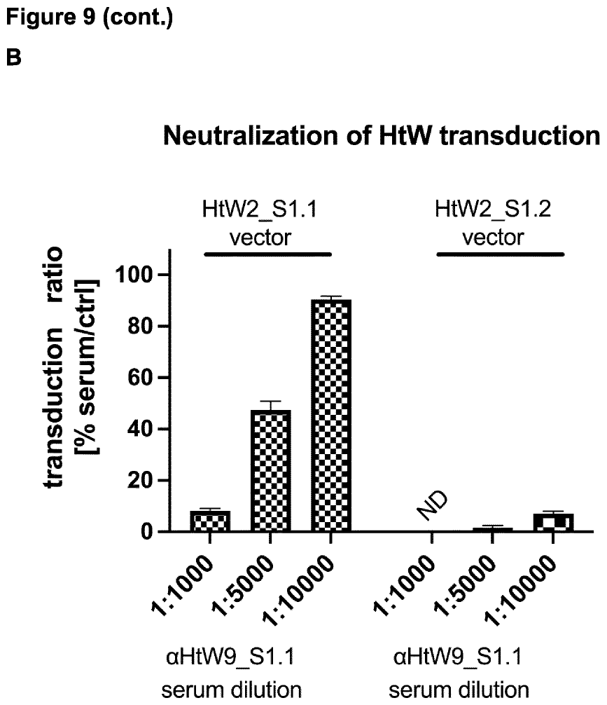

HtW2_S1.1 and HtW2_S1.2 vectors with sc-CMV-eGFP genome was pre-incubated at 37° C. with different dilutions (1:1000, 1:5000, 1:10000) of serum obtained by immunization of rabbits with HtW9_S1.1 empty capsids. These preincubated HtW vector/serum dilutions were then used to transduce HEK293T stably expressing ACE2 (+ACE2 cells) at an MOI of 250. The cells were imaged after 48h and analysed using brightfield and epifluorescence microscopy (EvosFL, Thermo Fisher Scientific) (FIG. 9A). After imaging at 48 h, the cells were collected and the fraction of eGFP-positive cells was analysed using a Countess II FL Automated Cell Counter (Thermo Fisher Scientific). At 1:1000 dilution the serum resulted in a strong or even complete neutralization of HtW_S1.1 and HtW_S1.2 vector, respectively, evident as a lack of eGFP signal. Neutralization was stronger against the HtW_S1.2 vector.

### Example 9: Structural Models of AAV Capsid and AAV Viral Protein (VP1) Comprising an scFv

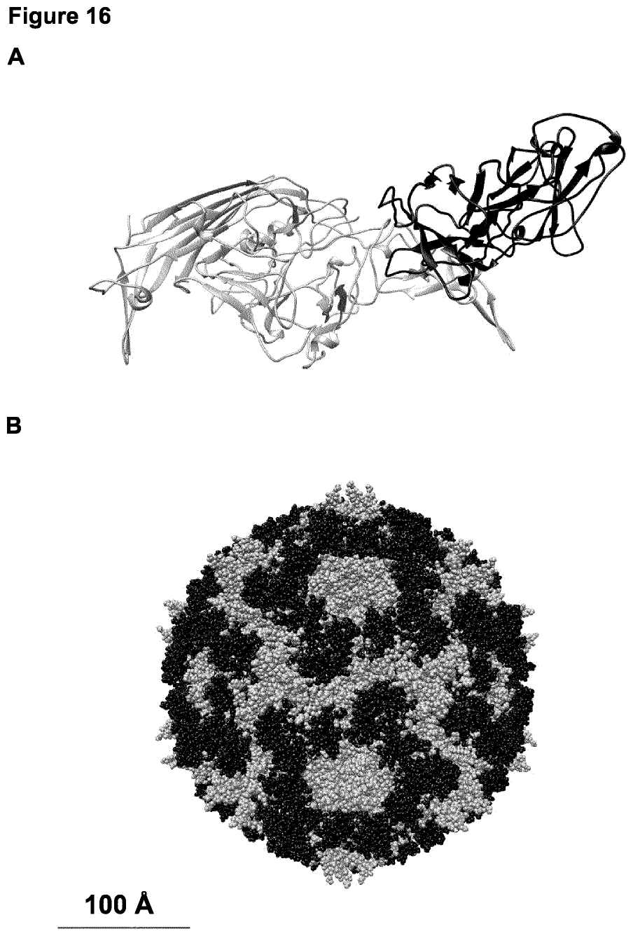

In addition to portions of the spike (S) protein of SARS-CoV-2, which serves as an immunogenic protein and as a protein comprising a binding domain, an anti-GFP scFv antibody fragment (SEQ ID NO: 73) has been cloned into VP1 of AAV at I-587. The structure of VP3 of AAV2 (SEQ ID NO: 2) and the resulting AAV capsid of AAV2 comprising the anti-GFP scFv antibody fragment (AAV2_αGFP scFv) (insert: SEQ ID NO: 73, AAV2 VP1 with insert: SEQ ID NO: 74) has been modelled using RoseTTAFold (https://robetta.bakerlab.org/) based de novo prediction of protein structure (FIG. 16A). The corresponding predicted 60-mer capsid structure of AAV2-αGFP is shown in FIG. 16B. As may be taken from the model, the large >200 amino acid anti-GFP scFv did not compromise the principle gross capsid structure. This indicates that in addition to the binding portion of the viral receptor SARS-CoV-2 Spike protein, also proteins comprising an antigen-binding domain, such as scFv, can be introduced into the capsid of AAV to specifically retarget the vector.

### Sequence Listing:

| SEQ ID NO: | |
|---|---|
| 1 | CAP AAV1 |
| 2 | CAP AAV2 |
| 3 | CAP AAV3 |
| 4 | CAP AAV4 |
| 5 | CAP AAV5 |
| 6 | CAP AAV6 |
| 7 | CAP AAV7 |
| 8 | CAP AAV8 |
| 9 | CAP AAV9 |
| 10 | CAP AAV10 |
| 11 | AA 333 to 529 of SARS-COV-2 S protein |
| | (YP 009724390.1) |
| 12 | AA 330 to 583 of SARS-COV-2 S protein |
| | (YP _009724390.1) |
| 13 | HtW2_S1.1 Cap protein sequence |
| 14 | HtW9_S1.1 Cap protein sequence |
| 15 | Full length SARS-COV-2 S protein (YP_009724390.1) |
| 16 | AAV2 marked 450-460 |
| 17 | AAV2 marked 585-592 |
| 18 | AAV9 marked 450-460 |
| 19 | AAV9 marked 585-592 |
| 20 | AAV1 marked 450-460 |
| 21 | AAV1 marked 585-592 |
| 22 | AAV8 marked 450-460 |
| 23 | AAV8 marked 585-592 |
| 24 | pHtW2_S1.1_fw |
| 25 | pHtW2_S1.1_rv |
| 26 | pHtW9_S1.1 fw |
| 27 | pHtW9_S1.1_rv |
| 28 | pHtW9_S1.1_BB_fw |
| 29 | pHtW9_S1.1_BB_rv |
| 30 | Human ACE2 Receptor (NP_068576.1) |
| 31 | Sars-Cov-2 S spike protein |
| | AA 6 to 255 of YP_009724390.1 |
| 32 | Sars-Cov-2 S spike protein |
| | AA 111 to 255 of YP_009724390.1 |
| 33 | Sars-Cov-2 S spike protein |
| | AA 187 to 261 of YP_009724390.1 |
| 34 | Sars-Cov-2 S spike protein |
| | AA 213 to 466 of YP_009724390.1 |
| 35 | Sars-Cov-2 S spike protein |
| | AA 213 to 363 of YP_009724390.1 |
| 36 | Sars-Cov-2 S spike protein |
| | AA 289 to 583 of YP_009724390.1 |
| 37 | Sars-Cov-2 S spike protein |
| | AA 300 to 558 of YP_009724390.1 |
| 38 | Sars-Cov-2 S spike protein |
| | AA 300 to 507 of YP_009724390.1 |
| 39 | Sars-Cov-2 S spike protein |
| | AA 300 to 462 of YP_009724390.1 |
| 40 | Sars-Cov-2 S spike protein |
| | AA 293 to 367 of YP_009724390.1 |
| 41 | Sars-Cov-2 S spike protein |
| | AA 659 to 916 of YP_009724390.1 |
| 42 | Sars-Cov-2 S spike protein |
| | AA 659 to 836 of YP_009724390.1 |
| 43 | Sars-Cov-2 S spike protein |
| | AA 703 to 902 of YP_009724390.1 |
| 44 | Sars-Cov-2 S spike protein |
| | AA 701 to 775 of YP_009724390.1 |
| 45 | Sars-Cov-2 S spike protein |
| | AA 763 to 837 of YP_009724390.1 |
| 46 | Sars-Cov-2 S spike protein |
| | AA 956 to 1205 of YP_009724390.1 |
| 47 | Sars-Cov-2 S spike protein |
| | AA 990 to 1189 of YP_009724390.1 |
| 48 | Sars-Cov-2 S spike protein |
| | AA 949 to 1023 of YP_009724390.1 |
| 49 | Sars-Cov-2 S spike protein |
| | AA 1038 to 1112 of YP_009724390.1 |
| 50 | Sars-Cov-2 S spike protein |
| | AA 1151 to 1228 of YP_009724390.1 |
| 51 | SARS-COV-2 N protein (YP_009724397.2) |
| 52 | Coronavirus envelop protein |
| | (E protein)(YP_009724392.1) |
| 53 | Coronavirus membrane glycoprotein (M protein) |
| | (YP_009724393.1) |
| 54 | EEIAIILASFSASTS (nsp2) |
| | (Epitope of the ORF1ab polyprotein (replicase |
| | complex); YP_009724389.1) |
| 55 | QTFFKLVNKFLALCA (nsp2) |
| | (Epitope of the ORF1ab polyprotein (replicase |
| | complex); YP_009724389.1) |
| 56 | KHFYWFFSNYLKRRV (nsp4) |
| | (Epitope of the ORF1ab polyprotein (replicase |
| | complex); YP_009724389.1) |
| 57 | NHNFLVQAGNVQLRV (nsp5) |
| | (Epitope of the ORF1ab polyprotein (replicase |
| | complex); YP_009724389.1) |
| 58 | NRYFRLTLGVYDYLV (nsp6) |
| | (Epitope of the ORF1ab polyprotein (replicase |
| | complex); YP_009724389.1) |
| 59 | KLLKSIAATRGATVV (nsp12) |
| | (Epitope of the ORF1ab polyprotein (replicase |
| | complex); YP_009724389.1) |
| 60 | NVNRFNVAITRAKVG (nsp13) |
| | (Epitope of the ORF1ab polyprotein (replicase |
| | complex); YP_009724389.1) |
| 61 | TSHKLVLSVNPYVCN (nsp13) |
| | (Epitope of the ORF1ab polyprotein (replicase |
| | complex); YP_009724389.1) |
| 62 | AA 434 to 724 of YP_009724389.1 (nsp2) |
| | comprising SEQ ID NO: 54 and SEQ ID NO: 55 |
| 63 | AA 3099 to 3387 of YP_009724389.1 (nsp4 and nsp5) |
| | comprising SEQ ID NO: 56 and SEQ ID NO: 57 |
| 64 | AA 3617 to 3902 of YP_009724389.1 (nsp6) |
| | comprising SEQ ID NO: 58 |
| 65 | AA 4848 to 5130 of YP_009724389.1 (nsp12) |
| | comprising SEQ ID NO: 59 |
| 66 | AA 5228 to 5516 of YP_009724389.1 (nsp13) |
| | comprising SEQ ID NO: 61 |
| 67 | AA 5732 to 6023 of YP_009724389.1 (nsp13) |
| | comprising SEQ ID NO: 60 |
| 68 | Coronavirus membrane glycoprotein ORF1ab |
| | polyprotein (replicase complex) (YP_009724389.1) |
| 69 | Sars-Cov-2 S spike protein |
| | AA 300 to 505 of YP_009724390.1 |
| 70 | HtW2_S1.2 Cap protein sequence |
| 71 | pHtW2_S1.2 fw |
| 72 | pHtW2_S1.2_rv |
| 73 | aGFP-scFv |
| 74 | AAV2_αGFP-scFv |

1. An adeno-associated virus (AAV) or a adeno-associated virus-like particle (AAVLP) comprising an insert of about 75-400 amino acids in the viral proteins (VPs) forming the capsid at an insertion site (I) at the top of variable region VIII and/or variable region IV (VR-VIII and/or VR-IV) of the VPs, wherein the insert is optionally flanked by a linker comprising one or more amino acids on one or both sides, preferably selected from the group consisting of A (Ala), G (Gly), S (Ser), T (Thr), L (Leu) and combinations thereof.

2. The AAV or AAVLP according to claim 1, wherein (a) the top of VR-VIII corresponds to amino acids 585 to 592 (I-585 to I-592) of VP1 of AAV 1, 2, 3, 6, 7, 8, 9 or 10 having the amino acid sequence of SEQ ID NO: 1, 2, 3, 6, 7, 8, 9 or 10, respectively, to amino acids 583 to 589 of VP1 of AAV 4 having the amino sequence of SEQ ID NO: 4, or to amino acids 574 to 580 of VP1 of AAV 5 having the amino sequence of SEQ ID NO: 5, and/or (b) the top of VR-IV corresponds to amino acids 450 to 460 (I-450 to I-460) of VP1 of AAV 1, 2, 3, 6, 7, 8, 9 or 10 having the amino acid sequence of SEQ ID NO: 1, 2, 3, 6, 7, 8, 9 or 10, respectively, to amino acids 445 to 455 (I-445 to I-455) of VP1 of AAV 4 having the amino sequence of SEQ ID NO: 4, or to amino acids 439 to 449 (I-439 to I-449) of VP1 of AAV 5 having the amino sequence of SEQ ID NO: 5.

3. The AAV or AAVLP according to claim 1, wherein the AAV or AAVLP is derived from AAV serotype 1 (AAV1), 2 (AAV2), 8 (AAV8) or 9 (AAV9), preferably wherein
(a) the insertion site is between two amino acids corresponding to amino acid position 587 and 588 (AAV2 I-587) or 588 and 589 (AAV2 I-588) and/or 453 and 454 (AAV2 I-453) of AAV2 VP1 having the amino acid sequence of SEQ ID NO: 2, preferably AAV2 I-587 or AAV2 I-588 or AAV2 I-453, more preferably AAV2 I-587 or AAV2 I-588;
(b) the insertion site is between two amino acids corresponding to amino acid position 587 and 588 (AAV1 I-587), 588 and 589 (AAV1 I-588) or 589 and 590 (AAV1 I-589) and/or 454 and 455 (AAV1 I-454), 455 and 456 (AAV1 I-455) or 456 and 457 (AAV1 I-456) having the amino acid sequence of SEQ ID NO: 1;
(c) the insertion site is between two amino acids corresponding to amino acid position 588 and 589 (AAV8 I-588) or 589 and 590 (AAV8 I-589) and/or 455 and 456 (I-455), 456 and 457 (I-456) or 457 and 458 (I-457) of AAV8 VP1 having the amino acid sequence of SEQ ID NO: 8, or
(d) the insertion site is between two amino acids corresponding to amino acid position 588 and 589 (AAV9 I-588) or 589 and 590 (AAV9 I-589) and/or 454 and 455 (I-454), 455 and 456 (I-455) or 456 and 457 (I-456) of AAV9 VP1 having the amino acid sequence of SEQ ID NO: 9.

4. The AAV or AAVLP according to claim 1, wherein the AAV or AAVLP has a capsid of about 60 VPs, wherein the VPs are
(a) VP3;
(b) VP1 and VP3; or
(c) VP1, VP2 and VP3 proteins, preferably at a ratio of 1:1:10.

5. The AAV or AAVLP according to claim 1, wherein the AAV comprises an ITR-flanked genome and is infectious, preferably wherein the ITR-flanked genome comprises a transgene.

6. The AAV or AAVLP according to claim 1, wherein the insert is (a) an immunogenic protein or a portion thereof and/or (b) a protein comprising a binding domain.

7. The AAV or AAVLP according to claim 6, wherein the insert is an immunogenic protein or a portion thereof and wherein
(a) the AAV comprises an ITR-flanked genome and is infectious, optionally wherein the ITR-flanked genome comprises a transgene encoding a further immunogenic protein or a portion thereof;
(b) an immunogenic protein or a portion thereof is inserted at the top of VR-VIII and at the top of VR-IV and wherein the immunogenic protein or a portion thereof inserted at the top of VR-VIII and the immunogenic protein or a portion thereof inserted at the top of VR-IV are the same or different; and/or
(c) the AAV or AAVLP is formed by 2 or more viral proteins comprising different inserts of a least about 75-300 amino acids, wherein the different inserts are each an immunogenic protein or a portion thereof, either an immunogenic protein or an immunogenic portion from a different protein or a different immunogenic portion from the same protein.

8. The AAV or AAVLP according to claim 6, wherein the immunogenic protein or the portion thereof is a viral, a bacterial or a parasitic protein or a portion thereof, and/or the immunogenic protein or the portion thereof is a tumor antigen.

9. The AAV or AAVLP according to claim 8, wherein the immunogenic protein or the portion thereof is
(a) a portion of coronavirus spike (S) protein;
(b) a portion of the SARS-CoV-2 spike (S) protein, preferably wherein the portion of the SARS-CoV-2 spike (S) protein comprises the SARS-CoV-2 S protein receptor binding domain (RBD) or a portion thereof; and/or
(c) a portion of the SARS-CoV-2 S protein comprising an amino acid sequence of SEQ ID NO: 11, 12, 31, 32, 33, 34, 35, 36, 37, 38, 39, 40, 41, 42, 43, 44, 45, 46, 47, 48, 49, 50 or 69, preferably 11, 12, 34, 35, 36, 37, 38, 42, or 69.

10. The AAV or AAVLP according to claim 1, wherein the insert is a protein comprising a binding domain and wherein the AAV comprises an ITR-flanked genome and is infectious and the ITR-flanked genome comprises a transgene.

11. The AAV or AAVLP according to claim 1, wherein the insert is a protein comprising an antigen-binding domain, preferably wherein the protein comprising an antigen-binding domain is a single-domain antibody (sdAb), a single chain variable fragment (scFv) or an antibody mimetic.

12. A pharmaceutical composition comprising the AAV or AAVLP according to claim 1, further comprising at least one pharmaceutically acceptable excipient.

13. A vaccine comprising the AAV or AAVLP according to claim 1, wherein the insert is an immunogenic protein or a portion thereof

14. A method to treat or prevent a disease induced by a virus, a bacterium or a parasite, comprising administering to a subject in need thereof a pharmaceutical composition comprising the AAV or AAVLP of claim 1, wherein the insert is an immunogenic protein or a portion thereof, and wherein the immunogenic protein or the portion thereof is an immunogenic protein of said virus, bacterium or parasite, respectively.

15. A therapy method comprising administering to a subject in need thereof the AAV or AAVLP according to claim 10, wherein the therapy is gene therapy.

16. A method for producing an AAV or an AAVLP, comprising the steps of
(i) preparing a cell comprising at least one DNA sequence comprising a cap gene and a rep gene, at least one DNA sequence comprising adenoviral helper sequences and optionally at least one DNA sequence comprising an ITR-flanked genome;
wherein the cap gene encodes a protein comprising an insert of about 75-400 amino acids in the viral proteins (VPs) forming the capsid at an insertion site (I) at the top of variable region VIII and/or variable region IV (VR-VIII and/or VR-IV) of the VPs, wherein the insert is optionally flanked by a linker comprising one or more amino acids on both sides, preferably selected from the group consisting of A (Ala), G (Gly), S (Ser), T (Thr), L (Leu) and combinations thereof;
(ii) cultivating the cells under conditions allowing the production of the AAV or the AAVLP; and
(iii) purifying the AAV or the AAVLP.

17. A method to treat or prevent a cancer, comprising administering to a subject in need thereof, a pharmaceutical composition comprising the AAV or AAVLP of claim 1, wherein the insert is an immunogenic protein or a portion thereof, and wherein the immunogenic protein or the portion thereof is a tumor antigen or portion thereof.


SEQUENCE LISTING (74 sequences)

SEQ ID NO: 1 - protein, 736 aa, Adeno-associated virus - 1
  AAV1 Cap Sequence (AAD27757)
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, Adeno-associated virus - 2 [sequence omitted]

SEQ ID NO: 3 - protein, 736 aa, Adeno-associated virus - 3
  AAV3B Cap Sequence
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, Adeno-associated virus - 4
  AAV4 Cap Sequence
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - protein, 724 aa, Adeno-associated virus - 5
  AAV5 Cap Sequence
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 736 aa, Adeno-associated virus - 6
  AAV6 Cap Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 737 aa, Adeno-associated virus - 7
  AAV7 Cap Sequence
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 8 - protein, 738 aa, Adeno-associated virus - 8
  AAV8 Cap Sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 736 aa, Adeno-associated virus - 9
  AAV9 Cap Sequence (Q6JC40)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 738 aa, Adeno-associated virus - 10
  AAV10 Cap Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 11 - protein, 197 aa, SARS coronavirus 2
  AA 333 to 529 of of SARS-CoV-2 S protein (YP_009724390.1) (S1.1))
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 12 - protein, 254 aa, SARS coronavirus 2
  aa 330 to 583 of SARS-CoV-2 S protein (YP_009724390.1)
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 13 - protein, 938 aa, Artificial Sequence
  HtW2_S1.1 Cap Protein Sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 937 aa, Artificial Sequence
  HtW9_S1.1 Cap Protein Sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 1273 aa, SARS coronavirus 2
  Full length SARS-CoV-2 S protein (YP_009724390.1)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 11 aa, Adeno-associated virus - 2
  AAV2 marked 450-460
TPSGTTTQSRL

SEQ ID NO: 17 - protein, 8 aa, Adeno-associated virus - 2
  AAV2 marked 585-592
RGNRQAAT

SEQ ID NO: 18 - protein, 11 aa, Adeno-associated virus - 9
  AAV9 marked 450-460
TINGSGQNQQT

SEQ ID NO: 19 - protein, 8 aa, Adeno-associated virus - 9
  AAV9 marked 585-592
QSAQAQAQ

SEQ ID NO: 20 - protein, 11 aa, Adeno-associated virus - 1
  AAV1 marked 450-460
QNQSGSAQNKD

SEQ ID NO: 21 - protein, 8 aa, Adeno-associated virus - 1
  AAV1 marked 585-592
QSSSTDPA

SEQ ID NO: 22 - protein, 11 aa, Adeno-associated virus - 8
  AAV8 marked 450-460
RTQTTGGTANT

SEQ ID NO: 23 - protein, 8 aa, Adeno-associated virus - 8
  AAV8 marked 585-592
NLQQQNTA

SEQ ID NO: 24 - DNA, 60 bp, Artificial Sequence
  pHtW2_S1.1_fw
ctaccaacctccagagaggcaacgcggccgcaactaatctttgtccgttcggtgaggttt

SEQ ID NO: 25 - DNA, 55 bp, Artificial Sequence
  pHtW2_S1.1_rv
tgacatctgcggtagctgcttggcgcgccgctccctttttgggcccacaaactgt

SEQ ID NO: 26 - DNA, 60 bp, Artificial Sequence
  pHtW9_S1.1_fw
ccacaaaccaccagagtgcccaagcggccgcaactaatctttgtccgttcggtgaggttt

SEQ ID NO: 27 - DNA, 55 bp, Artificial Sequence
  pHtW9_S1.1_rv
tttgaacccaaccggtctgcgcctgtgccgcctttttgggcccacaaactgtagc

SEQ ID NO: 28 - DNA, 33 bp, Artificial Sequence
  pHtW9_S1.1_BB_fw
gcacaggcgcagaccggttgggttcaaaaccaa

SEQ ID NO: 29 - DNA, 23 bp, Artificial Sequence
  pHtW9_S1.1_BB_rv
ttgggcactctggtggtttgtgg

SEQ ID NO: 30 - protein, 805 aa, Homo sapiens [sequence omitted]

SEQ ID NO: 31 - protein, 250 aa, SARS coronavirus 2
  AA 6 to 255 of YP_009724390.1
VLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSS

SEQ ID NO: 32 - protein, 145 aa, SARS coronavirus 2
  AA 111 to 255 of YP_009724390.1
DSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSS

SEQ ID NO: 33 - protein, 75 aa, SARS coronavirus 2
  AA 187 to 261 of YP_009724390.1
KNLREFVFKNIDGYFKIYSKHTPINLVRDLPQGFSALEPLVDLPIGINITRFQTLLALHRSYLTPGDSSSGWTAG

SEQ ID NO: 34 - protein, 254 aa, SARS coronavirus 2
  AA 213 to 466 of YP_009724390.1
VRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFER

SEQ ID NO: 35 - protein, 151 aa, SARS coronavirus 2
  AA 213 to 363 of YP_009724390.1
VRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVA

SEQ ID NO: 36 - protein, 295 aa, SARS coronavirus 2
  AA 289 to 583 of YP_009724390.1
VDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 37 - protein, 259 aa, SARS coronavirus 2
  AA 300 to 558 of YP_009724390.1
KCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKK

SEQ ID NO: 38 - protein, 208 aa, SARS coronavirus 2
  AA 300 to 507 of YP_009724390.1
KCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQP

SEQ ID NO: 39 - protein, 163 aa, SARS coronavirus 2
  AA 300 to 462 of YP_009724390.1
KCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLK

SEQ ID NO: 40 - protein, 75 aa, SARS coronavirus 2
  AA 293 to 367 of YP_009724390.1
LDPLSETKCTLKSFTVEKGIYQTSNFRVQPTESIVRFPNITNLCPFGEVFNATRFASVYAWNRKRISNCVADYSV

SEQ ID NO: 41 - protein, 258 aa, SARS coronavirus 2
  AA 659 to 916 of YP_009724390.1
SYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVL

SEQ ID NO: 42 - protein, 178 aa, SARS coronavirus 2
  AA 659 to 836 of YP_009724390.1
SYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQ

SEQ ID NO: 43 - protein, 200 aa, SARS coronavirus 2
  AA 703 to 902 of YP_009724390.1
NSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQM

SEQ ID NO: 44 - protein, 75 aa, SARS coronavirus 2
  AA 701 to 775 of YP_009724390.1
AENSVAYSNNSIAIPTNFTISVTTEILPVSMTKTSVDCTMYICGDSTECSNLLLQYGSFCTQLNRALTGIAVEQD

SEQ ID NO: 45 - protein, 75 aa, SARS coronavirus 2
  AA 763 to 837 of YP_009724390.1
LNRALTGIAVEQDKNTQEVFAQVKQIYKTPPIKDFGGFNFSQILPDPSKPSKRSFIEDLLFNKVTLADAGFIKQY

SEQ ID NO: 46 - protein, 250 aa, SARS coronavirus 2
  AA 956 to 1205 of YP_009724390.1
AQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGK

SEQ ID NO: 47 - protein, 200 aa, SARS coronavirus 2
  AA 990 to 1189 of YP_009724390.1
EVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEV

SEQ ID NO: 48 - protein, 75 aa, SARS coronavirus 2
  AA 949 to 1023 of YP_009724390.1
QDVVNQNAQALNTLVKQLSSNFGAISSVLNDILSRLDKVEAEVQIDRLITGRLQSLQTYVTQQLIRAAEIRASAN

SEQ ID NO: 49 - protein, 75 aa, SARS coronavirus 2
  AA 1038 to 1112 of YP_009724390.1
KRVDFCGKGYHLMSFPQSAPHGVVFLHVTYVPAQEKNFTTAPAICHDGKAHFPREGVFVSNGTHWFVTQRNFYEP

SEQ ID NO: 50 - protein, 78 aa, SARS coronavirus 2
  AA 1151 to 1228 of YP_009724390.1
ELDKYFKNHTSPDVDLGDISGINASVVNIQKEIDRLNEVAKNLNESLIDLQELGKYEQYIKWPWYIWLGFIAGLIAIV

SEQ ID NO: 51 - protein, 419 aa, SARS coronavirus 2
  SARS-CoV-2 N protein (YP_009724397.2)
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 52 - protein, 75 aa, SARS coronavirus 2
  Coronavirus envelop protein (E protein) (YP_009724392.1)
MYSFVSEETGTLIVNSVLLFLAFVVFLLVTLAILTALRLCAYCCNIVNVSLVKPSFYVYSRVKNLNSSRVPDLLV

SEQ ID NO: 53 - protein, 222 aa, SARS coronavirus 2
  Coronavirus membrane glycoprotein (M protein) (YP_009724393.1)
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 54 - protein, 15 aa, SARS coronavirus 2
  (nsp2) Epitope of the ORF1ab polyprotein (replicase complex) (YP_009724389.1)
EEIAIILASFSASTS

SEQ ID NO: 55 - protein, 15 aa, SARS coronavirus 2
  (nsp2) Epitope of the ORF1ab polyprotein (replicase complex) (YP_009724389.1)
QTFFKLVNKFLALCA

SEQ ID NO: 56 - protein, 15 aa, SARS coronavirus 2
  (nsp4) Epitope of the ORF1ab polyprotein (replicase complex) (YP_009724389.1)
KHFYWFFSNYLKRRV

SEQ ID NO: 57 - protein, 15 aa, SARS coronavirus 2
  (nsp5) Epitope of the ORF1ab polyprotein (replicase complex) (YP_009724389.1)
NHNFLVQAGNVQLRV

SEQ ID NO: 58 - protein, 15 aa, SARS coronavirus 2
  (nsp6) Epitope of the ORF1ab polyprotein (replicase complex) (YP_009724389.1)
NRYFRLTLGVYDYLV

SEQ ID NO: 59 - protein, 15 aa, SARS coronavirus 2
  (nsp12) Epitope of the ORF1ab polyprotein (replicase complex) (YP_009724389.1)
KLLKSIAATRGATVV

SEQ ID NO: 60 - protein, 15 aa, SARS coronavirus 2
  (nsp13) Epitope of the ORF1ab polyprotein (replicase complex) (YP_009724389.1)
NVNRFNVAITRAKVG

SEQ ID NO: 61 - protein, 15 aa, SARS coronavirus 2
  (nsp13) Epitope of the ORF1ab polyprotein (replicase complex) (YP_009724389.1)
TSHKLVLSVNPYVCN

SEQ ID NO: 62 - protein, 291 aa, SARS coronavirus 2
  AA 434 to 724 of YP_009724389.1 (nsp2)
NHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSR

SEQ ID NO: 63 - protein, 289 aa, SARS coronavirus 2
  AA 3099 to 3387 of YP_009724389.1 (nsp4 and nsp5)
SFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSG

SEQ ID NO: 64 - protein, 286 aa, SARS coronavirus 2
  AA 3617 to 3902 of YP_009724389.1 (nsp6)
GIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAK

SEQ ID NO: 65 - protein, 283 aa, SARS coronavirus 2
  AA 4848 to 5130 of YP_009724389.1 (nsp12)
YRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVD

SEQ ID NO: 66 - protein, 289 aa, SARS coronavirus 2
  AA 5228 to 5516 of YP_009724389.1 (nsp13)
RILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSK

SEQ ID NO: 67 - protein, 292 aa, SARS coronavirus 2
  AA 5732 to 6023 of YP_009724389.1 (nsp13)
PRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATR

SEQ ID NO: 68 - protein, 7096 aa, SARS coronavirus 2 [sequence omitted]

SEQ ID NO: 69 - protein, 206 aa, SARS coronavirus 2
  AA 300 to 505 of YP_009724390.1 (S1.2)
KCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGY

SEQ ID NO: 70 - protein, 946 aa, Artificial Sequence
  HtW2_S1.2 Cap Protein Sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAAKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 71 - DNA, 59 bp, Artificial Sequence
  pHtW2_S1.2_fw
tatctaccaacctccagagaggcaacgcggcggcgaagtgcaccctgaagagcttcacc

SEQ ID NO: 72 - DNA, 55 bp, Artificial Sequence
  pHtW2_S1.2_rv
ttgacatctgcggtagctgcttggcgcgccgcgtatcccactccgttggttggct

SEQ ID NO: 73 - protein, 248 aa, Artificial Sequence
  aGFP-scFv
EVQLQQSGPELVKPGSSMKISCKASGYSFTGYTMNWVKQSHGQNLEWIGLINPYNGGTNYNQKFKGKATLTVDKSSSTAYMELLGLTSEDSAVYYCTRGNSDFSAWFAYWGQGTSVTVSSGGGGSGGGGSGGGGSGGGGSDIQLTQSPAIMSPSLGERVTMTCTASSSVGSSYLHWFQQKPGSSPKLWIYSTSNLASGVPARFSGSGSGTSYSLTISRMEAEDAATYYCHQYHRTPYTFGGGTKLEIK

SEQ ID NO: 74 - protein, 988 aa, Artificial Sequence
  AAV2_aGFP-scFv
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAAEVQLQQSGPELVKPGSSMKISCKASGYSFTGYTMNWVKQSHGQNLEWIGLINPYNGGTNYNQKFKGKATLTVDKSSSTAYMELLGLTSEDSAVYYCTRGNSDFSAWFAYWGQGTSVTVSSGGGGSGGGGSGGGGSGGGGSDIQLTQSPAIMSPSLGERVTMTCTASSSVGSSYLHWFQQKPGSSPKLWIYSTSNLASGVPARFSGSGSGTSYSLTISRMEAEDAATYYCHQYHRTPYTFGGGTKLEIKAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL